(19) United States
(12) Patent Application Publication
(10) Pub. No.: US 2024/0190931 A1
(43) Pub. Date: Jun. 13, 2024

(54) MAGEC2 IMMUNOGENIC PEPTIDES, BINDING PROTEINS RECOGNIZING MAGEC2 IMMUNOGENIC PEPTIDES, AND USES THEREOF

(71) Applicants: TScan Therapeutics, Inc., Waltham, MA (US); Academisch Ziekenhuis Leiden, Leiden (NL)

(72) Inventors: Andrew P. Ferretti, Hopkinton, MA (US); Yifan Wang, Arlington, MA (US); Gavin MacBeath, Wakefield, MA (US); Qikai Xu, Chestnut Hill, MA (US)

(21) Appl. No.: 18/286,728

(22) PCT Filed: Apr. 14, 2022
(86) PCT No.: PCT/US22/24728

§ 371 Date: Oct. 12, 2023

Related U.S. Application Data
(60) Provisional application No. 63/329,523, filed on Apr. 11, 2022.
(60) Provisional application No. 63/174,808, filed on Apr. 14, 2021.

(51) Int. Cl. C07K 14/47; A61K 38/00; C07K 14/74; C12N 15/63; G01N 33/68

(52) U.S. Cl. CPC C07K 14/4748; C07K 14/70539; C12N 15/63; G01N 33/6854; A61K 38/00; G01N 2800/52

(57) ABSTRACT

Provided herein are MAGEC2 immunogenic peptides, binding proteins recognizing MAGEC2 immunogenic peptides, and uses thereof.

### CROSS-REFERENCE TO RELATED APPLICATIONS

This application claims the benefit of U.S. Provisional Application No. 63/174,808, filed on 14 Apr. 2021, and U.S. Provisional Application No. 63/329,523, filed on 11 Apr. 2022; the entire contents of each of said applications are incorporated herein in their entirety by this reference.

### BACKGROUND OF THE INVENTION

Adoptive cell transfer (ACT) using engineered T cells has demonstrated great efficacy in treating certain types of liquid tumor and holds promise for treating solid tumor. T cell receptor-engineered T cells (TCR-T) are T cells expressing an exogenous TCR that recognizes an antigen that exist in cancer cells. The TCR-antigen interaction is the core component of the targeting mechanism that allows the TCR-T cells to kill cancer cells. One of the challenges for the broad testing and adoption of TCR-T therapy is the lack of TCR-antigen pairs that are applicable to a wide range of patients and indications. Although several TCR and antigens have been explored in clinical trials, most pursued antigens are restricted to one HLA allele, namely HLA-A*02:01, which limits the number of patients who are eligible for the therapy and also allowing cancer cells to develop potential resistance by mutating the HLA-A*02:01 allele.

In addition, the number of pursued antigens is limited due to the difficulty of discovering novel TCR-antigen pairs which commonly require prediction of the MHC presented epitope. However, such epitopes may not be immunogenic, thereby making it difficult to identify a reactive TCR, or the epitope may not be processed and presented physiologically by the cancer cells. Accordingly, there is a great need in the art to identify TCR-antigen pairs in the context of a variety of widely applicable HLA alleles in order to develop useful reagents to diagnose, prognose, treat, and screen agents relevant for disorders characterized by the expression of the antigens.

### SUMMARY OF THE INVENTION

The present invention is based, at least in part, on the discovery of MAGEC2 immunogenic peptides and binding proteins recognizing such MAGEC2 immunogenic peptides based on unbiased functional screens used to discover the antigen of TCR clonotypes identified from subjects having disorders associated with MAGEC2 expression (e.g., subjects afflicted with as melanoma, head & neck cancer, lung cancer, cervical cancer, prostate cancer, multiple myeloma, hepatocellular carcinoma, breast invasive carcinoma, or bladder urothelial carcinoma). The identified TCRs recognized MAGEC2 immunogenic peptides, such as those listed in Table 1, in the context of a variety of HLA alleles (e.g., HLA-B*07:02 and HLA-A*24:02). MAGEC2 is demonstrated herein to be selectively expressed in cancer and testis tissue, but not in normal somatic tissues, thereby making it an ideal target for ACT. The ability of MAGEC2 binding proteins (e.g., TCRs described herein) to bind MAGEC2 immunogenic peptides and to elicit immune responses that kill cells expressing MAGEC2 (e.g., cancer cells) demonstrates the utility of such binding proteins in a diversity of uses, including methods of diagnosis, prognosis, treatment, and screening of agents relevant for disorders characterized by MAGEC2 expression.

In one aspect, an immunogenic peptide comprising a peptide epitope selected from peptide sequences listed in Table 1, is provided.

In another aspect, an immunogenic peptide consisting of a peptide epitope selected from peptide sequences listed in Table 1, is provided.

Numerous embodiments are further provided that may be applied to any aspect encompassed by the present invention and/or combined with any other embodiment described herein. For example, in one embodiment, the immunogenic peptide is derived from a MAGEC2 protein, optionally wherein the immunogenic peptide is 8, 9, 10, 11, 12, 13, 14, 15, 16, 17, 18, 19, 20, 21, 22, 23, 24, or 25 amino acids in length. In another embodiment, the immunogenic peptide is capable of eliciting an immune response against MAGEC2 and/or MAGEC2-expressing cells in a subject, optionally wherein the immune response is i) a T cell response and/or a CD8+ T cell response and/or ii) selected from the group consisting of T cell expansion, cytokine release, and/or cytotoxic killing.

In still another aspect, an immunogenic composition comprising at least one immunogenic peptide described herein, is provided.

Numerous embodiments are further provided that may be applied to any aspect encompassed by the present invention and/or combined with any other embodiment described herein. For example, in one embodiment, the immunogenic composition further comprises an adjuvant. In another embodiment, the immunogenic composition is capable of eliciting an immune response against MAGEC2 and/or MAGEC2-expressing cells in a subject, optionally wherein the immune response is i) a T cell response and/or a CD8+ T cell response and/or ii) selected from the group consisting of T cell expansion, cytokine release, and/or cytotoxic killing.

In yet another aspect, a composition comprising a peptide epitope selected from peptide sequences listed in Table 1, and an MHC molecule, is provided.

Numerous embodiments are further provided that may be applied to any aspect encompassed by the present invention and/or combined with any other embodiment described herein. For example, in one embodiment, the MHC molecule is an MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

In another aspect, a stable MHC-peptide complex, comprising an immunogenic peptide described herein in the context of an MHC molecule, is provided.

Numerous embodiments are further provided that may be applied to any aspect encompassed by the present invention and/or combined with any other embodiment described herein. For example, in one embodiment, the MHC molecule is an MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore.

In still another aspect, an immunogenic composition comprising a stable MHC-peptide complex described herein, and an adjuvant, is provided.

In yet another aspect, an isolated nucleic acid that encodes an immunogenic peptide described herein, or a complement thereof, is provided.

In another aspect, a vector comprising an isolated nucleic acid described herein, is provided.

In still another aspect, a cell that a) comprises an isolated nucleic acid described herein, b) comprises a vector described herein, and/or c) produces one or more immunogenic peptides described herein and/or presents at the cell surface one or more stable MHC-peptide complexes described herein, optionally wherein the cell is genetically engineered, is provided.

In yet another aspect, a device or kit comprising a) one or more immunogenic peptides described herein and/or b) one or more stable MHC-peptide complexes described herein, said device or kit optionally comprising a reagent to detect binding of a) and/or b) to a binding protein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

In another aspect, a method of detecting T cells that bind a stable MHC-peptide complex comprising: a) contacting a sample comprising T cells with a stable MHC-peptide complex described herein; and b) detecting binding of T cells to the stable MHC-peptide complex, optionally further determining the percentage of stable MHC-peptide-specific T cells that bind to the stable MHC-peptide complex, optionally wherein the sample comprises peripheral blood mononuclear cells (PBMCs), is provided.

Numerous embodiments are further provided that may be applied to any aspect encompassed by the present invention and/or combined with any other embodiment described herein. For example, in one embodiment, T cells are CD8+ T cells. In another embodiment, detecting and/or determining is performed using fluorescence activated cell sorting (FACS), enzyme linked immunosorbent assay (ELISA), radioimmune assay (RIA), immunochemically, Western blot, or intracellular flow assay. In still another embodiment, a sample comprises T cells contacted with, or suspected of having been contacted with, one or more MAGEC2 proteins or fragments thereof.

In still another aspect, a method of determining whether a T cell has had exposure to MAGEC2 comprising: a) incubating a cell population comprising T cells with an immunogenic peptide described herein or a stable MHC-peptide complex described herein; and b) detecting the presence or level of reactivity, wherein the presence of or a higher level of reactivity compared to a control level indicates that the T cell has had exposure to MAGEC2, optionally wherein the cell population comprising T cells is obtained from a subject, is provided.

In yet another aspect, a method for predicting the clinical outcome of a subject afflicted with a disorder characterized by MAGEC2 expression comprising: a) determining the presence or level of reactivity between T cells obtained from the subject and one more immunogenic peptides described herein or one or more stable MHC-peptide complexes described herein; and b) comparing the presence or level of reactivity to that from a control, wherein the control is obtained from a subject having a good clinical outcome, wherein the presence or a higher level of reactivity in the subject sample as compared to the control indicates that the subject has a good clinical outcome, is provided.

In another aspect, a method of assessing the efficacy of a therapy for a disorder characterized by MAGEC2 expression comprising: a) determining the presence or level of reactivity between T cells obtained from the subject and one more immunogenic peptides described herein or one or more stable MHC-peptide complexes described herein, in a first sample obtained from the subject prior to providing at least a portion of the therapy to the subject, and b) determining the presence or level of reactivity between the one more immunogenic peptides described herein, or the one or more stable MHC-peptide complexes described herein, and T cells obtained from the subject present in a second sample obtained from the subject following provision of the therapy to the subject, wherein the presence or a higher level of reactivity in the second sample, relative to the first sample, is an indication that the therapy is efficacious for treating the disorder characterized by MAGEC2 expression in the subject, is provided.

Numerous embodiments are further provided that may be applied to any aspect encompassed by the present invention and/or combined with any other embodiment described herein. For example, in one embodiment, the level of reactivity is indicated by a) the presence of binding and/or b) T cell activation and/or effector function, optionally wherein the T cell activation or effector function is T cell proliferation, killing, or cytokine release. In another embodiment, a method further comprises repeating steps a) and b) at a subsequent point in time, optionally wherein the subject has undergone treatment to ameliorate the disorder characterized by MAGEC2 expression between the first point in time and the subsequent point in time. In still another embodiment, T cell binding, activation, and/or effector function is detected using fluorescence activated cell sorting (FACS), enzyme linked immunosorbent assay (ELISA), radioimmune assay (RIA), immunochemically, Western blot, or intracellular flow assay. In yet another embodiment, a control level is a reference number. In another embodiment, a control level is a level of a subject without the disorder characterized by MAGEC2 expression.

In still another aspect, a method of preventing and/or treating a disorder characterized by MAGEC2 expression in a subject comprising administering to the subject a therapeutically effective amount of a composition described herein.

In yet another aspect, a method of identifying a peptide-binding molecule, or antigen-binding fragment thereof, that binds to a peptide epitope selected from the peptide sequences listed in Table 1 comprising: a) providing a cell presenting a peptide epitope selected from the peptide sequences listed in Table 1 in the context of an MHC molecule on the surface of the cell; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

Numerous embodiments are further provided that may be applied to any aspect encompassed by the present invention and/or combined with any other embodiment described herein. For example, in one embodiment, a step a) comprises contacting the MHC molecule on the surface of the cell with a peptide epitope selected from the peptide sequences listed in Table 1. In another embodiment, a step a) comprises expressing the peptide epitope selected from the peptide sequences listed in Table 1 in the cell using a vector comprising a heterologous sequence encoding the peptide epitope.

In another aspect, a method of identifying a peptide-binding molecule or antigen-binding fragment thereof that binds to a peptide epitope selected from the peptide sequences listed in Table 1 comprising: a) providing a peptide epitope either alone or in a stable MHC-peptide complex, comprising a peptide epitope selected from the peptide sequences listed in Table 1, either alone or in the context of an MHC molecule; b) determining binding of a plurality of candidate peptide-binding molecules or antigen-binding fragments thereof to the peptide or stable MHC-peptide complex; and c) identifying one or more peptide-binding molecules or antigen-binding fragments thereof that bind to the peptide epitope or the stable MHC-peptide complex, optionally wherein the MHC or MHC-peptide complex is as described herein, is provided.

Numerous embodiments are further provided that may be applied to any aspect encompassed by the present invention and/or combined with any other embodiment described herein. For example, in one embodiment, a plurality of candidate peptide binding molecules comprises an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain. In another embodiment, a plurality of candidate peptide binding molecules comprises at least 2, 5, 10, 100, 10³, 10⁴, 10⁵, 10⁶, 10⁷, 10⁸, 10⁹, or more, different candidate peptide binding molecules. In still another embodiment, a plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject. In yet another embodiment, a subject or population of subjects are a) not afflicted with a disorder characterized by MAGEC2 expression and/or have recovered from a disorder characterized by MAGEC2 expression, or b) are afflicted with a disorder characterized by MAGEC2 expression. In another embodiment, a subject or population of subjects has been administered a composition described herein. In still another embodiment, a subject is an animal model of a disorder characterized by MAGEC2 expression and/or a mammal, optionally wherein the mammal is a human, a primate, or a rodent. In yet another embodiment, a subject is an animal model of a disorder characterized by MAGEC2 expression, an HLA-transgenic mouse, and/or a human TCR transgenic mouse. In another embodiment, a sample comprises peripheral blood mononuclear cells (PBMCs), T cells, and/or CD8+ memory T cells.

In still another aspect, a peptide-binding molecule or antigen-binding fragment thereof identified according to a method described herein, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

In yet another aspect, a method of treating a disorder characterized by MAGEC2 expression in a subject comprising administering to the subject a therapeutically effective amount of genetically engineered T cells that express a peptide-binding molecule or antigen-binding fragment thereof that i) binds to a peptide epitope selected from the sequences listed in Table 1, ii) is identified according to a method described herein, and/or iii) binds to a stable MHC-peptide complex comprising a peptide epitopes selected from the sequences listed in Table 1 in the context of an MHC molecule, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, optionally wherein the MHC or MHC-peptide complex is as described herein, is provided.

Numerous embodiments are further provided that may be applied to any aspect encompassed by the present invention and/or combined with any other embodiment described herein. For example, in one embodiment, T cells are isolated from a) the subject, b) a donor not afflicted with the disorder characterized by MAGEC2 expression, or c) a donor recovered from a disorder characterized by MAGEC2 expression.

In another aspect, a method of treating a disorder characterized by MAGEC2 expression in a subject comprising transfusing antigen-specific T cells to the subject, wherein the antigen-specific T cells are generated by: a) stimulating immune cells from a subject with a composition described herein; and b) expanding antigen-specific T cells in vitro or ex vivo, optionally i) isolating immune cells from the subject before stimulating the immune cells and/or ii) wherein the immune cells comprise PBMCs, T cells, CD8+ T cells, naive T cells, central memory T cells, and/or effector memory T cells, is provided.

Numerous embodiments are further provided that may be applied to any aspect encompassed by the present invention and/or combined with any other embodiment described herein. For example, in one embodiment, agents are placed in contact under conditions and for a time suitable for the formation of at least one immune complex between the peptide epitope, immunogenic peptide, stable MHC-peptide complex, T cell receptor, and/or immune cells. In another embodiment, a peptide epitope, immunogenic peptide, stable MHC-peptide complex, and/or T cell receptor is expressed by cells and the cells are expanded and/or isolated during one or more steps. In still another embodiment, a disorder characterized by MAGEC2 expression is a cancer or relapse thereof, optionally wherein the cancer is selected from the group consisting of melanoma, head & neck cancer, lung cancer, cervical cancer, prostate cancer, multiple myeloma, hepatocellular carcinoma, breast invasive carcinoma, and bladder urothelial carcinoma. In yet another embodiment, a subject is an animal model of a disorder characterized by MAGEC2 expression and/or a mammal, optionally wherein the mammal is a human, a primate, or a rodent.

In still another aspect, a binding protein that binds a polypeptide comprising an immunogenic peptide sequence described herein, an immunogenic peptide described herein, and/or the stable MHC-peptide complex described herein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

Numerous embodiments are further provided that may be applied to any aspect encompassed by the present invention and/or combined with any other embodiment described herein. For example, in one embodiment, a binding protein comprises: a) a T cell receptor (TCR) alpha chain CDR sequence with at least about 80% identity to a TCR alpha chain CDR sequence selected from the group consisting of TCR alpha chain CDR sequences listed in Table 2; and/or b) a TCR beta chain CDR sequence with at least about 80% identity to a TCR beta chain CDR sequence selected from the group consisting of TCR beta chain CDR sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M. In another embodiment, a binding protein comprises: a) a TCR alpha chain variable (V_{α}) domain sequence with at least about 80% identity to a TCR V_{α} domain sequence selected from the group consisting of TCR V_{α} domain sequences listed in Table 2; and/or b) a TCR beta chain variable (V_{β}) domain sequence with at least about 80% identity to a TCR V_{β} domain sequence selected from the group consisting of TCR V_{β} domain sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M. In still another embodiment, a binding protein comprises: a) a TCR alpha chain sequence with at least about 80% identity to a TCR alpha chain sequence selected from the group consisting of TCR alpha chain sequences listed in Table 2; and/or b) a TCR beta chain sequence with at least about 80% identity to a TCR beta chain sequence selected from the group consisting of TCR beta chain sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M. In yet another embodiment, a binding protein comprises: a) a TCR alpha chain CDR sequence selected from the group consisting of TCR alpha chain CDR sequences listed in Table 2; and/or b) a TCR beta chain CDR sequence selected from the group consisting of TCR beta chain CDR sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M. In yet another embodiment, a binding protein comprises: a) a TCR alpha chain variable (V_{α}) domain sequence selected from the group consisting of TCR V_{α} domain sequences listed in Table 2; and/or b) a TCR beta chain variable (V_{β}) domain sequence selected from the group consisting of TCR Vp domain sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M, is provided. In another embodiment, a binding protein comprises: a) a TCR alpha chain sequence selected from the group consisting of TCR alpha chain sequences listed in Table 2; and/or b) a TCR beta chain sequence selected from the group consisting of TCR beta chain sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M, is provided. In another embodiment, 1) a TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) a TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2. In still another embodiment, a binding protein is chimeric, humanized, or human. In yet another embodiment, a binding protein comprises a binding domain having a transmembrane domain, and an effector domain that is intracellular. In another embodiment, a TCR alpha chain and a TCR beta chain are covalently linked, optionally wherein the TCR alpha chain and the TCR beta chain are covalently linked through a linker peptide. In still another embodiment, a TCR alpha chain and/or a TCR beta chain are covalently linked to a moiety, optionally wherein the covalently linked moiety comprises an affinity tag or a label. In yet another embodiment, an affinity tag is selected from the group consisting of aCD34 enrichment tag, glutathione-S-transferase (GST), calmodulin binding protein (CBP), protein C tag, Myc tag, HaloTag, HA tag, Flag tag, His tag, biotin tag, and V5 tag, and/or wherein the label is a fluorescent protein. In another embodiment, a covalently linked moiety is selected from the group consisting of an inflammatory agent, cytokine, toxin, cytotoxic molecule, radioactive isotope, or antibody or antigen-binding fragment thereof. In still another embodiment, a binding protein binds to the pMHC complex on a cell surface. In yet another embodiment, an MHC or MHC-peptide complex is as described herein. In another embodiment, binding of a binding protein to the MAGEC2 peptide-MHC (pMHC) complex elicits an immune response, optionally wherein the immune response is i) a T cell response and/or a CD8+ T cell response and/or ii) selected from the group consisting of T cell expansion, cytokine release, and/or cytotoxic killing. In still another embodiment, a binding protein is capable of specifically and/or selectively binding to a MAGEC2 immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M. In yet another embodiment, a binding protein has a higher binding affinity to the peptide-MHC (pMHC) than does a known T-cell receptor, optionally wherein the higher binding affinity is at least 1.05-fold higher. In another embodiment, a binding protein induces higher T cell expansion, cytokine release, and/or cytotoxic killing than does a known T-cell receptor when contacted with target cells with a heterozygous expression of MAGEC2, optionally wherein the induction is at least 1.05-fold higher. As used herein, references to fold changes, in some embodiments, may be in comparison to any reference modality of interest, such as comparison to a different binding protein; comparison to the same binding protein under different context like expression of the same binding protein in a different immune cell, at a different level, in combination with other agents described herein; and the like. In still another embodiment, cytotoxic killing is of a target cancer cell. In yet another embodiment, cancer is selected from the group consisting of melanoma, head & neck cancer, lung cancer, cervical cancer, prostate cancer, multiple myeloma, hepatocellular carcinoma, breast invasive carcinoma, and bladder urothelial carcinoma. In another embodiment, a binding protein does not bind to a peptide-MHC (pMHC) complex selected from the group consisting of ALKDVEERV/HLA-A*02, LLFGLALIEV/HLA-A*02, SESIKKKVL/HLA-B*44, and ASSTLYLVF/HLA-B*57.

In yet another aspect, a TCR alpha chain and/or beta chain selected from the group consisting of TCR alpha chain and beta chain sequences listed in Table 2, is provided.

In another aspect, an isolated nucleic acid molecule i) that hybridizes, under stringent conditions, with the complement of a nucleic acid encoding a polypeptide selected from the group consisting of polypeptide sequences listed in Table 2, ii) a sequence with at least about 80% homology to a nucleic acid encoding a polypeptide selected from the group consisting of the polypeptide sequences listed in Table 2, and/or iii) ii) a sequence with at least about 80% homology to a nucleic acid encoding listed in Table 2, optionally wherein the isolated nucleic acid molecule comprises 1) a TRAV, TRAJ, and/or TRAC gene or fragment thereof selected from the group of TRAV, TRAJ, and TRAC genes listed in Table 2 and/or 2) a TRBV, TRBJ, and/or TRBC gene or fragment thereof selected from the group of TRBV, TRBJ, and TRBC genes listed in Table 2, is provided.

Numerous embodiments are further provided that may be applied to any aspect encompassed by the present invention and/or combined with any other embodiment described herein. For example, in one embodiment, a nucleic acid is codon optimized for expression in a host cell.

In still another aspect, a vector comprising an isolated nucleic acid described herein, optionally wherein i) the vector is a cloning vector, expression vector, or viral vector and/or ii) the vector comprises a vector sequence listed in Table 3, is provided.

Numerous embodiments are further provided that may be applied to any aspect encompassed by the present invention and/or combined with any other embodiment described herein. For example, in one embodiment, a vector further comprises a nucleic acid sequence encoding CD8a and/or CD8P. In another embodiment, a nucleic acid sequence encoding CD8a or CD8P is operably linked to a nucleic acid encoding a tag. In still another embodiment, a nucleic acid encoding a tag is at the 5′ upstream of the nucleic acid sequence encoding CD8a or CD8P such that the tag is fused to the N-terminus of CD8a or CD8P. In yet another embodiment, a tag is a CD34 enrichment tag. In another embodiment, an isolated nucleic acid described herein and a nucleic acid sequence encoding CD8a and/or CD8P are interconnected with an internal ribosome entry site or a nucleic acid sequence encoding a self-cleaving peptide. In still another embodiment, a self-cleaving peptide is P2A, E2A, F2A or T2A.

In yet another aspect, a host cell which comprises an isolated nucleic acid described herein, comprises a vector described herein, and/or expresses a binding protein described herein, optionally wherein the cell is genetically engineered, is provided.

Numerous embodiments are further provided that may be applied to any aspect encompassed by the present invention and/or combined with any other embodiment described herein. For example, in one embodiment, a host cell comprises a chromosomal gene knockout of a TCR gene, an HLA gene, or both. In another embodiment, a host cell comprises a knockout of an HLA gene selected from an α1 macroglobulin gene, α2 macroglobulin gene, α3 macroglobulin gene, β1 microglobulin gene, β2 microglobulin gene, and combinations thereof. In still another embodiment, a host cell comprises a knockout of a TCR gene selected from a TCR α variable region gene, TCR β variable region gene, TCR constant region gene, and combinations thereof. In yet another embodiment, a host cell expresses CD8α and/or CD8β, optionally wherein the CD8α and/or CD8β is fused to a CD34 enrichment tag. In another embodiment, host cells are enriched using the CD34 enrichment tag. In still another embodiment, a host cell is a hematopoietic progenitor cell, peripheral blood mononuclear cell (PBMC), cord blood cell, or immune cell. In yet another embodiment, an immune cell is a T cell, cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or a combination thereof. In yet another embodiment a T cell is a naive T cell, central memory T cell, effector memory T cell, or a combination thereof. In another embodiment, a T cell is a primary T cell or a cell of a T cell line. In still another embodiment, a T cell does not express or has a lower surface expression of an endogenous TCR. In yet another embodiment, a host cell is capable of producing a cytokine or a cytotoxic molecule when contacted with a target cell that comprises a peptide-MHC (pMHC) complex comprising a MAGEC2 peptide epitope in the context of an MHC molecule. In another embodiment, a host cell is contacted with the target cell in vitro, ex vivo, or in vivo. In still another embodiment, a cytokine is TNF-α, IL-2, and/or IFN-γ. In yet another embodiment, a cytotoxic molecule is perforins and/or granzymes, optionally wherein the cytotoxic molecule is granzyme B. In another embodiment, a host cell is capable of producing a higher level of cytokine or a cytotoxic molecule when contacted with a target cell with a heterozygous expression of MAGEC2. In still another embodiment, a host cell is capable of producing an at least 1.05-fold higher level of cytokine or a cytotoxic molecule. In yet another embodiment, a host cell is capable of killing a target cell that comprises a peptide-MHC (pMHC) complex comprising the MAGEC2 peptide epitope in the context of an MHC molecule. In another embodiment, killing is determined by a killing assay. In still another embodiment, a ratio of the host cell and the target cell in the killing assay is from 20:1 to 1:4. In yet another embodiment, a target cell is a target cell pulsed with 1 μg/mL to 50 μg/mL of MAGEC2 peptide, optionally wherein the target cell is a cell monoallelic for an MHC matched to the MAGEC2 peptide. In another embodiment, a host cell is capable of killing a higher number of target cells when contacted with target cells with a heterozygous expression of MAGEC2, optionally wherein the cell killing is at least 1.05-fold higher. In still another embodiment, a target cell is cell line or a primary cell, optionally wherein the target cell is selected from the group consisting of a HEK293 derived cell line, a cancer cell line, a primary cancer cell, a transformed cell line, and an immortalized cell line. In yet another embodiment, a MAGEC2 immunogenic peptide is as described herein and/or wherein an MHC or MHC-peptide complex is as described herein. In another embodiment, a host cell does not induce T cell expansion, cytokine release, or cytotoxic killing when contact with a target cell that comprises a peptide-MHC (pMHC) complex selected from the group consisting of ALKDVEERV/HLA-A*02, LLFGLALIEV/HLA-A*02, SESIKKKVL/HLA-B*44, and ASSTLYLVF/HLA-B*57. In still another embodiment, a host cell does not express MAGEC2 antigen, is not recognized by a binding protein described herein, is not of serotype HLA-B*07, does not express an HLA-B*07 allele, is not of serotype HLA-A*24, and/or does not express an HLA-A*24 allele.

In another aspect, a population of host cells described herein, is provided.

In still another aspect, a composition comprising a) a binding protein described herein, b) an isolated nucleic acid described herein, c) a vector described herein, d) a host cell described herein, and/or e) a population of host cells described herein, and a carrier, is provided.

In yet another aspect, a device or kit comprising a) a binding protein described herein, b) an isolated nucleic acid described herein, c) a vector described herein, d) a host cell described herein, and/or e) a population of host cells described herein, said device or kit optionally comprising a reagent to detect binding of a), d) and/or e) to a pMHC complex, is provided.

In another aspect, a method of producing a binding protein described herein, wherein the method comprises the steps of: (i) culturing a transformed host cell which has been transformed by a nucleic acid comprising a sequence encoding a binding protein described herein under conditions suitable to allow expression of said binding protein; and (ii) recovering the expressed binding protein, is provided.

In still another aspect, a method of producing a host cell expressing a binding protein described herein, wherein the method comprises the steps of: (i) introducing a nucleic acid comprising a sequence encoding a binding protein described herein into the host cell; and (ii) culturing the transformed host cell under conditions suitable to allow expression of said binding protein, is provided.

In yet another aspect, a method of detecting the presence or absence of a MAGEC2 antigen and/or a cell expressing MAGEC2, optionally wherein the cell is a hyperproliferative cell, comprising detecting the presence or absence of said MAGEC2 antigen in a sample by use of at least one binding protein described herein, at least one host cell described herein, or a population of host cells described herein, wherein detection of the MAGEC2 antigen is indicative of the presence of a MAGEC2 antigen and/or cell expressing MAGEC2, is provided.

Numerous embodiments are further provided that may be applied to any aspect encompassed by the present invention and/or combined with any other embodiment described herein. For example, in one embodiment, at least one binding protein, or at least one host cell, forms a complex with the MAGEC2 peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay. In another embodiment, a method further comprises obtaining a sample from a subject.

In another aspect, a method of detecting the level of a disorder characterized by MAGEC2 expression in a subject, comprising: a) contacting a sample obtained from the subject with at least one binding protein described herein, at least one host cell described herein, or a population of host cells described herein; and b) detecting the level of reactivity, wherein the presence or a higher level of reactivity compared to a control level indicates the level of the disorder characterized by MAGEC2 expression in the subject, is provided.

Numerous embodiments are further provided that may be applied to any aspect encompassed by the present invention and/or combined with any other embodiment described herein. For example, in one embodiment, a control level is a reference number. In another embodiment, a control level is a level from a subject without the disorder characterized by MAGEC2 expression.

In still another aspect, a method for monitoring the progression of a disorder characterized by MAGEC2 expression in a subject, the method comprising: a) detecting in a subject sample the presence or level of reactivity between a sample obtained from the subject and at least one binding protein described herein, at least one host cell described herein, or a population of host cells described herein; b) repeating step a) at a subsequent point in time; and c) comparing the level of MAGEC2 or the cell of interest expressing MAGEC2 detected in steps a) and b) to monitor the progression of the disorder characterized by MAGEC2 expression in the subject, wherein an absent or reduced MAGEC2 level or the cell of interest expressing MAGEC2 detected in step b) compared to step a) indicates an inhibited progression of the disorder characterized by MAGEC2 expression in the subject and a presence or increased MAGEC2 level or the cell of interest expressing MAGEC2 detected in step b) compared to step a) indicates a progression of the disorder characterized by MAGEC2 expression in the subject, is provided.

Numerous embodiments are further provided that may be applied to any aspect encompassed by the present invention and/or combined with any other embodiment described herein. For example, in one embodiment, a subject has undergone treatment to treat a disorder characterized by MAGEC2 expression between the first point in time and the subsequent point in time.

In yet another aspect, a method for predicting the clinical outcome of a subject afflicted with a disorder characterized by MAGEC2 expression comprising: a) determining the presence or level of reactivity between a sample obtained from the subject and at least one binding protein described herein, at least one host cell described herein, or a population of host cells described herein; and b) comparing the presence or level of reactivity to that from a control, wherein the control is obtained from a subject having a good clinical outcome; wherein the absence or a reduced level of reactivity in the subject sample as compared to the control indicates that the subject has a good clinical outcome, is provided.

In another aspect, a method of assessing the efficacy of a therapy for a disorder characterized by MAGEC2 expression comprising: a) determining the presence or level of reactivity between a sample obtained from the subject and at least one binding protein described herein, at least one host cell described herein, or a population of host cells described herein, in a first sample obtained from the subject prior to providing at least a portion of the therapy for the disorder characterized by MAGEC2 expression to the subject, and b) determining the presence or level of reactivity between a sample obtained from the subject and at least one binding protein described herein, at least one host cell described herein, or a population of host cells described herein, in a second sample obtained from the subject following provision of the therapy for the disorder characterized by MAGEC2 expression, wherein the absence or a reduced level of reactivity in the second sample, relative to the first sample, is an indication that the therapy is efficacious for treating the disorder characterized by MAGEC2 expression in the subject, and wherein the presence or an increased level of reactivity in the second sample, relative to the first sample, is an indication that the therapy is not efficacious for treating the disorder characterized by MAGEC2 expression in the subject, is provided.

Numerous embodiments are further provided that may be applied to any aspect encompassed by the present invention and/or combined with any other embodiment described herein. For example, in one embodiment, a level of reactivity is indicated by a) the presence of binding and/or b) T cell activation and/or effector function, optionally wherein the T cell activation or effector function is T cell proliferation, killing, or cytokine release. In another embodiment, a T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

In still another aspect, a method of preventing and/or treating a disorder characterized by MAGEC2 expression comprising contacting target cells expressing MAGEC2 with a therapeutically effective amount of a composition comprising cells expressing at least one binding protein described herein, optionally wherein the composition is administered to a subject, is provided.

Numerous embodiments are further provided that may be applied to any aspect encompassed by the present invention and/or combined with any other embodiment described herein. For example, in one embodiment, a cell is an allogeneic cell, syngeneic cell, or autologous cell. In another embodiment, a cell is host cell described herein or a population of host cells described herein. In still another embodiment, a target cell is a cancer cell expressing MAGEC2. In yet another embodiment, a cell composition further comprises a pharmaceutically acceptable carrier. In another embodiment, a cell composition induces an immune response against the target cell expressing MAGEC2 in the subject. In still another embodiment, a cell composition induces an antigen-specific T cell immune response against the target cell expressing MAGEC2 in the subject. In yet another embodiment, an antigen-specific T cell immune response comprises at least one of a CD4⁺ helper T lymphocyte (Th) response and a CD8+ cytotoxic T lymphocyte (CTL) response. In another embodiment, a method further comprises administering at least one additional treatment for the disorder characterized by MAGEC2 expression, optionally wherein the at least one additional treatment for the disorder characterized by MAGEC2 expression is administered concurrently or sequentially with the composition. In still another embodiment, a disorder characterized by MAGEC2 expression is a cancer or relapse thereof, optionally wherein the cancer is selected from the group consisting of melanoma, head & neck cancer, lung cancer, cervical cancer, prostate cancer, multiple myeloma, hepatocellular carcinoma, breast invasive carcinoma, and bladder urothelial carcinoma. In yet another embodiment, a subject is an animal model of a disorder characterized by MAGEC2 expression and/or a mammal, optionally wherein the mammal is a human, a primate, or a rodent.

### BRIEF DESCRIPTION OF THE DRAWINGS

FIG. 1A and FIG. 1B show nucleic acid gene expression of MAGEC2 in a wide panel of tumor types and normal tissues (FIG. 1A), as well as relative to tumor-associated biomarkers MAGEA4 and NY-ESO-1, across a representative set of tumor types (FIG. 1B).

FIG. 2A-FIG. 2C demonstrate the identification of MAGEC2 antigenic peptides. Overlapping peptide sequences from MAGEC2 were evaluated for predicted binding to HLA-B*07:02. The top predicted MHC binding peptide was synthesized and used to pulse HLA-B*07:02-expressing HEK293T cells. Reactivity of a pool of TCR-transduced T cells was measured using the activation markers, CD137 and CD69. FIG. 2A shows results of a pool of TCR-transduced T cells co-cultured with HLA-B*07:02 monoallelic HEK293T cells expressing a granzyme-activated fluorescent reporter and an overlapping library of 60-mer peptides spanning 1,670 cancer testis antigens. Tiles with a fold enrichment>4 with identical overlapping peptide sequences area highlighted with dots corresponding to the underlying shared gene. FIG. 2B shows representative sequences of MAGEC2 peptides identified in screening data. Overlapping sequences (solid box) were analyzed for HLA-B*07:02-predicted binding to identify a top predicted binder having a sequence RAREFMELL (termed the “RAR” peptide, dashed box). FIG. 2C shows reactivity of the TCR-transduced T cell pool to the identified HLA-B*07:02 binding peptides for the top two enriched genes in the screening data.

FIG. 3A and FIG. 3B show identification of MAGEC2-reactive TCRs from pools of TCR-transduced T cells. Target cells were pulsed with peptide from MAGEC2 (RAR) and co-cultured with a pool of TCR-transduced T cells. Reactive TCRs were identified by sequencing the exogenous TCR from sorted CD137/CD69 double-positive cells. FIG. 3A shows difference in fold enrichment of sorted TCRs reacting to RAR- and SPQ-pulsed cells over the input library. FIG. 3B shows proportion of cells expressing the indicated TCRs in the sorted cells (magenta: TCR 8-3, purple: TCR 4-1, aqua: TCR 11-2).

FIG. 4A and FIG. 4B show that TCR 8-3 recognizes and kills RAR peptide-pulsed cells. FIG. 4A shows results of wild-type HEK293T cells pulsed with serial dilutions of the RAR peptide and co-cultured with T cells transduced with TCR 8-3. Reactivity of the TCR was measured by IFNγ release. FIG. 4B shows results of HLA-B*07:02 monoallelic HEK293T cells expressing a granzyme-activated fluorescent reporter pulsed with the indicated concentrations of RAR peptide and co-cultured with T cells transduced with TCR 8-3.

FIG. 5A-FIG. 5E show that TCR 8-3 recognizes melanoma cells that express MAGEC2. FIG. 5A shows a Western blot illustrating MAGEC2 protein levels in the indicated melanoma cell lines. FIG. 5B shows quantification of protein levels of MAGEC2 relative to GAPDH. FIG. 5C shows correspondence of MAGEC2 protein levels with MAGEC2 mRNA expression data from publicly available datasets (TRON cell line portal). FIG. 5D demonstrates reactivity of the 8-3 TCR to melanoma cell lines as measured by IFNγ release. FIG. 5E shows that reactivity of the 8-3 TCR corresponds to MAGEC2 expression.

FIG. 6 shows that TCR 8-3 kills MAGEC2-expressing melanoma cell lines. Indicated cell lines were transduced with Incucyte® NucLight™ Red and either co-cultured with TCR 8-3 (magenta), co-cultured with a MHC-mismatched control TCR (blue), or cultured without T cells (yellow). T cells were added at an E:T ratio of 2:1 at the start of the assay. Red fluorescence was measured overtime with an Incucyte® instrument and displayed as total red object count normalized to timepoint 0.

FIG. 7A-FIG. 7C demonstrate the identification of MAGEC2 antigenic peptides. Overlapping peptide sequences from MAGEC2 were evaluated for predicted binding to HLA-A*24:02. The top predicted MHC binding peptide was synthesized and used to pulse HLA-A*24:02-expressing HEK293T cells. Reactivity of a pool of TCR-transduced T cells was measured using the activation-induced markers (AIM), CD137 and CD69 (i.e., percentage of AIM double-positive cells; see FIG. 2 for additional assay details). FIG. 7A shows results of a pool of TCR-transduced T cells co-cultured with HLA-A*24:02 monoallelic HEK293T cells expressing a granzyme-activated fluorescent reporter and an overlapping library of 60-mer peptides spanning 1,670 cancer testis antigens. Tiles with a fold enrichment>4 with identical overlapping peptide sequences area highlighted with dots corresponding to the underlying shared gene. FIG. 7B shows representative sequences of MAGEC2 peptides identified in screening data. Overlapping sequences were analyzed for HLA-A*24:02-predicted binding to identify top predicted binder sequences. FIG. 7C shows reactivity of the TCR-transduced T cell pool to the identified HLA-A*24:02 binding peptides for the peptides identified in the screening data, including a peptide having the sequence VGPDHFCVF (termed the “VGP” peptide) (see also Table 1B).

FIG. 8A-FIG. 8C show identification of MAGEC2-reactive TCRs from pools of TCR-transduced T cells. Target cells were pulsed with peptide from MAGEC2 (VGP) and co-cultured with a pool of TCR-transduced T cells. Reactive TCRs were identified by sequencing the exogenous TCR from sorted CD137/CD69 double-positive cells. FIG. 8A shows difference in fold enrichment of sorted TCRs reacting to VGO-pulsed and unpulsed cells over the input library. FIG. 8B shows proportion of cells expressing the indicated TCRs in the sorted cells. FIG. 8C shows reactivity of TCR 4-58-transduced T cells measured using the activation-induced markers (AIM), CD137 and CD69 (i.e., percentage of AIM double-positive cells; see FIG. 2 for additional assay details).

FIG. 9A and FIG. 9B show that TCR 4-58 recognizes and kills VGP peptide-pulsed cells. FIG. 9A shows results of wild-type HEK293T cells pulsed with serial dilutions of the VGP peptide and co-cultured with T cells transduced with TCR 4-58. Reactivity of the TCR was measured by IFP+ reporter-based cell killing. FIG. 9B shows results of TCR 4-58 MAGEC2-expressing cell lines, such as melanoma cell lines. The indicated cell line was transduced with Incucyte® NucLight™ Red and either co-cultured with TCR 4-58 (magenta), co-cultured with untransduced control T cells (purple), or cultured without T cells (blue). T cells were added at an E:T ratio of 4:1 at the start of the assay. Red fluorescence was measured overtime with an Incucyte® instrument and displayed as total red object count normalized to timepoint 0.

For any figure showing a bar histogram, curve, or other data associated with a legend, the bars, cruve, or other data presented from left to right for each indication correspond directly and in order to the boxes from top to bottom, or from left to right, of the legend.

### DETAILED DESCRIPTION OF THE INVENTION

The present invention is based, at least in part, on the discovery of MAGEC2 immunogenic peptides (e.g., those comprising or consisting of sequences listed in Table 1), binding proteins (e.g., those having sequences listed in Table 2) that recognize MAGEC2 antigens, and uses thereof. A systematic, comprehensive survey was carried out to map the precise T cell targets recognized by an initial pool of T cells of interest.

Accordingly, the present invention relates, in part, to the identified epitopes (immunodominant peptides) of therapeutically relevant MAGEC2 protein and related compositions (e.g., immunodominant peptides, vaccines, and the like), compositions comprising immunogenic peptides alone or with MHC molecules, stable MHC-peptide complexes, methods of diagnosing, prognosing, and monitoring immune responses to disorders characterized by MAGEC2 expression, and methods for preventing and/or treating disorders characterized by MAGEC2 expression. The present invention also relates, in part, to identified binding proteins (e.g., TCRs), host cells expressing binding proteins (e.g., TCRs), compositions comprising binding proteins (e.g., TCRs) and host cells expressing binding proteins (e.g., TCRs), methods of diagnosing, prognosing, and monitoring T cell response to cells expressing MAGEC2, and methods for preventing and/or treating disorders characterized by MAGEC2 expression.

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element. In addition, references to a table provided herein encompass all sub-tables of the table unless otherwise indicated.

The term “administering” means providing a pharmaceutical agent or composition to a subject, and includes, but is not limited to, administering by a medical professional and self-administering. This involves the physical introduction of a composition comprising a therapeutic agent to a subject, using any of the various methods and delivery systems known to those skilled in the art. In some embodiments, routes of administration for binding proteins described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a binding protein described herein may be administered via a non-parenteral route, such as a topical, epidermal or mucosal route of administration, for example, intranasally, orally, vaginally, rectally, sublingually or topically. Administering may also be performed, for example, once, a plurality of times, and/or over one or more extended periods.

As used herein, the term “antigen” refers to any natural or synthetic immunogenic substance, such as a protein, peptide, or hapten. An antigen may be a MAGEC2 antigen, or a fragment thereof, against which protective or therapeutic immune responses are desired. An “epitope” is the part of the antigen bound by a natural or synthetic substance.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions may be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen presenting cell” or “APC” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “antigen-binding portion” of a binding protein, such as a TCR, as used herein, refers to one or more portions of a TCR that retain the ability to bind (e.g., specifically and/or selectively) to an antigen (e.g., a MAGEC2 antigen). Such portions are, for example, between about 8 and about 1,500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of a TCR can be performed by fragments of a full-length TCR Examples of binding portions encompassed within the term “antigen-binding portion” of a TCR, include (i) a Fv fragment consisting of the V_{α} and V_{β} domains of a TCR, (ii) an isolated complementarity determining region (CDR) or (iii) a combination of two or more isolated CDRs which may optionally be joined by a synthetic linker. Furthermore, although V_{α} and V_{β}, are coded by separate genes, they may be joined, using recombinant methods, by a synthetic linker that enables them to be made as a single protein chain in which the V_{α} and V_{β} regions pair to form monovalent molecules (known as single chain TCR (scTCR)). Such single chain TCRs are also intended to be encompassed within the term “antigen-binding portion” of a TCR These TCR fragments can be obtained using conventional techniques known to those with skill in the art, and the fragments are screened for utility in the same manner as are complete binding proteins. Antigen-binding portions may be produced by recombinant DNA techniques, or by enzymatic or chemical cleavage of intact immunoglobulins.

The terms “complementarity determining region” and “CDR” are synonymous with “hypervariable region” or “HVR” and are known in the art to refer to non-contiguous sequences of amino acids within certain binding proteins, such as TCR variable regions, which confer antigen specificity and/or binding affinity. For TCRs, in general, there are three CDRs in each α-chain variable region (αCDR1, αCDR2, and αCDR3) and three CDRs in each β-chain variable region (βCDR1, βCDR2, and βCDR3). CDR3 is believed to be the main CDR responsible for recognizing processed antigen. CDR1 and CDR2 mainly interact with the MHC.

The term “body fluid” refers to fluids that are excreted or secreted from the body as well as fluids that are normally not excreted or secreted from the body (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the body fluid comprises immune cells, optionally wherein the immune cells are cytotoxic lymphocytes such as cytotoxic T cells and/or NK cells, CD4+ T cells, and the like.

The term “coding region” refers to regions of a nucleotide sequence comprising codons that are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residues of the first portion are capable of base pairing with nucleotide residues in the second portion. In some embodiments, all nucleotide residues of the first portion are capable of base pairing with nucleotide residues in the second portion.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

“CD3” is known in the art as a multi-protein complex of six chains (see, Abbas and Lichtman, Cellular and Molecular Immunology (9^{th} Edition) (2018); Janeway et al. (Immunobiology) (9^{th} Edition) (2016)). In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is a characteristic that is believed to allow these chains to associate with positively charged regions or residues of T cell receptor chains. The intracellular tails of the CD3γ, CD3δ, and CD3ε chains each contain a single conserved motif known as an immunoreceptor tyrosine-based activation motif or IT AM, whereas each CD3ζ chain has three ITAMs. Without wishing to be bound by theory, it is believed that the IT AMs are important for the signaling capacity of a TCR complex. CD3 used in accordance with the present invention may be from various animal species, including human, mouse, rat, or other mammals.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ, or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

The term “chimeric antigen receptor” or “CAR” refers to a fusion protein that is engineered to contain two or more amino acid sequences linked together in a way that does not occur naturally or does not occur naturally in a host cell, which fusion protein can function as a receptor when present on a surface of a cell. CARs encompassed by the present invention include an extracellular portion comprising an antigen-binding domain (i.e., obtained or derived from an immunoglobulin or immunoglobulin-like molecule, such as a TCR specific for a MAGEC2 antigen, a single chain TCR-derived binding protein, an scFv derived from an antibody, an antigen binding domain derived or obtained from a killer immunoreceptor from an NK cell, and the like) linked to a transmembrane domain and one or more intracellular signaling domains (such as an effector domain, optionally containing co-stimulatory domain(s)) (see, e.g., Sadelain et al. (2013) Cancer Discov. 3:388; see also Harris and Kranz (2016) Trends Pharmacol. Sci. 37: 220; Stone et al. (2014) Cancer Immunol. Immunother. 63:1163).

As used herein, the term “cytotoxic T lymphocyte (CTL) response” refers to an immune response induced by cytotoxic T cells. CTL responses are mediated primarily by CD8⁺ T cells.

The term “consisting essentially of is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not

substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The term “determining a suitable treatment regimen for the subject” is taken to mean the determination of a treatment regimen (i.e., a single therapy or a combination of different therapies that are used for the prevention and/or treatment of the viral infection in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin−CD117⁺ phenotype or those found in the thymus (referred to as progenitor thymocytes).

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. In some embodiments, the first region comprises a first portion and the second region comprises a second portion, whereby, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residue positions of each of the portions are occupied by the same nucleotide residue. In some embodiments, all nucleotide residue positions of each of the portions are occupied by the same nucleotide residue.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system may be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity may be enhanced by at least 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 500%, or more.

The term “immunotherapeutic agent” may include any molecule, peptide, antibody or other agent which can stimulate a host immune system to generate an immune response to a viral infection in the subject Various immunotherapeutic agents are useful in the compositions and methods described herein.

The term “immune cell” refers to any cell of the immune system that originates from a hematopoietic stem cell in the bone marrow, which gives rise to two major lineages: a myeloid progenitor cell (which give rise to myeloid cells such as monocytes, macrophages, dendritic cells, megakaryocytes and granulocytes); and a lymphoid progenitor cell (which give rise to lymphoid cells such as T cells, B cells and natural killer (NK) cells). Exemplary immune system cells include a CD4⁺ T cell, a CD8⁺ T cell, a CD4 CD8 double negative T cell, a gd T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the binding protein, antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), or, in some embodiments, less than about 25%, 20%, 15%, 10%, 5%, 1%, or less, or any range in between inclusive, such as less than about 1% to 5%, of non-biomarker protein. When binding protein, antibody, polypeptide, peptide or fusion protein or fragment thereof, e.g., a biologically active fragment thereof, is recombinantly produced, it may be substantially free of culture medium, i.e., culture medium represents less than about 20%, 15%, 10%, 5%, 1%, or less, or any range in between inclusive, such as less than about 10% to 50%, of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular binding protein-antigen interaction. The binding affinity of binding proteins encompassed by the present invention may be measured or determined by standard binding protein-target binding assays, for example, competitive assays, saturation assays, or standard immunoassays, such as ELISA or RIA. A relatively lower K_{d} value indicates a relatively higher binding affinity (e.g., K_{d} values of less than or equal to about 5×10⁻⁴ M (500 uM) include a K_{d} value of 1×10⁻⁴ M (100 uM) and a 100 uM K_{d} indicates a relatively higher binding affinity as compared to a 500 uM K_{d}).

A “kit” is any manufacture (e.g., a package or container) comprising at least one reagent, e.g., a probe or small molecule, for detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In some embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

As used herein, the term “linked” refers to the association of two or more molecules. The linkage may be covalent or non-covalent. The linkage also may be genetic (i.e., recombinantly fused). Such linkages may be achieved using a wide variety of art recognized techniques, such as chemical conjugation and recombinant protein production.

A “linker,” in some embodiments, may refer to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In some embodiments, a linker is comprised of about two to about 35 amino acids, for instance, or about four to about 20 amino acids or about eight to about 15 amino acids or about 15 to about 25 amino acids.

The term “MAGEC2” refers to a particular member of the melanoma antigen gene family clustered on human chromosome Xq26-q27 that is also known as cancer/testis antigen 10 (CT10), hepatocellular cancer antigen 587 (HCA587), and melanoma antigen, family E, 1, cancer/testis specific (MAGEE1) (Gure et al. (2000) Int. J. Cancer 85:726-732; Li et al. (2003) Lab Invest. 83:1185-1192; Ma et al. (2004) Int. J. Cancer 109:698-702; Godelaine et al. (2007) Cancer Immunol. Immunother. 56:753-759; Reiner et al. (2009) Int. 20 J. Cancer 124:352-357; Doyle et al. (2010) Mol. Cell 39:93-974; von Boehmer et al. (2011) PLoS One 6, e21366; Wen et al. (2011) Cancer Sci. 102:1455-1461; de Carvalho et al. (2013) Cancer Immunol. Immunother. 62:191-195; Bhatia et al. (2013) J Invest. Dermatol. 133:759-767); Yang et al. (2014) Breast Cancer Res. Treat. 145:23-32; and Kunert et al. (2016) J Immunol. 197:2541-2552). MAGEC2 is believed to enhance ubiquitin ligase activity of RING-type zinc finger-containing E3 ubiquitin-protein ligases, such as by recruiting and/or stabilizing Ubl-conjugating enzymes (E2) at the E3:substrate complex. MAGEC2 enhances in vitro ubiquitin ligase activity of TRIM28 and stimulates p53/TP53 ubiquitination in presence of Ubl-conjugating enzyme UBE2H leading to p53/TP53 degradation. MAGEC2 is not expressed in normal tissues, except for testis, and is expressed in tumors of various histological types such as melanoma, head & neck cancer, lung cancer, cervical cancer, prostate cancer, multiple myeloma, hepatocellular carcinoma, breast invasive carcinoma, and bladder urothelial carcinoma.

The term “MAGEC2” is intended to include fragments, variants (e.g., allelic variants), and derivatives thereof. Representative human MAGEC2 cDNA and human MAGEC2 protein sequences are well-known in the art and are publicly available from the National Center for Biotechnology Information (NCBI) (see, for example, ncbi.nlm.nih.gov/gene/51438). For example, human MAGEC2 (NP_057333.1) is encodable by the transcript (NM_016249.4). Nucleic acid and polypeptide sequences of MAGEC2 orthologs in organisms other than humans are well-known and include, for example, chimpanzee MAGEC2 (NM_001302428.1, NP_001289357.1, XM_016942653.1, and XP_016798142.1) and rhesus monkey MAGEC2 (NM_001265825.1, NP_001252754.1, XM_028841693.1, and XP_028697526.1). Representative sequences of MAGEC2 sequences are also presented below in Table 3.

Anti-MAGEC2 antibodies suitable for detecting MAGEC2 protein are well-known in the art and include, for example, antibodies TA315476 and TA342769 (OriGene, Rockville, MD); antibodies orb353181 and orb 125944 (Biorbyt, Cambridge, United Kingdom); antibodies A05335 and A05335-1 (Boster Bio, Pleasanton, CA); and antibodies ABIN2788251 and ABIN2706502 (Antibodies-online, Limerick, PA). In addition, reagents are well-known for detecting MAGEC2 expression. Moreover, multiple siRNA, shRNA, CRISPR constructs for modulating MAGEC2 expression can be found in the commercial product lists of a variety of companies, such as open reading frame (ORF) clones SC07208 and RN211555 (OriGene, Rockville, MD) and CRISPR knockouts GA109805 and KN403064 (OriGene, Rockville, MD). It is to be noted that the term can further be used to refer to any combination of features described herein regarding MAGEC2 molecules. For example, any combination of sequence composition, percentage identify, sequence length, domain structure, functional activity, etc. can be used to describe a MAGEC2 molecule encompassed by the present invention.

The term “MAGEC2 antigen” or “MAGEC2 peptide antigen” or “MAGEC2-containing peptide antigen” or “MAGEC2 epitope” or “MAGEC2 peptide epitope” or “MAGEC2 peptide” refers to a naturally or synthetically produced immunogenic portion of MAGEC2. In some embodiments, MAGEC2 antigen protein can range in length from about 7, 8, 9, 10, 11, 12, 13, 14, 15, 16, 17, 18, 19, 20, 21, 22, 23, 24, 25 amino acids, or any range in between, inclusive, such as 8-15 amino acids. In some embodiments, MAGEC2 antigen protein can form a complex with an MHC (e.g., HLA) molecule such that a binding protein of this disclosure that recognizes a MAGEC2 peptide:MHC (e.g., HLA) complex can bind (e.g., specifically and/or selectively) to such a complex. Representative MAGEC2 peptide antigen sequences are shown in Table 1.

The term “major histocompatibility complex” (MHC) refers to glycoproteins that deliver peptide antigens to a cell surface. MHC class I molecules are heterodimers having a membrane spanning a chain (with three a domains) and a non-covalently associated b2 microglobulin. MHC class II molecules are composed of two transmembrane glycoproteins, a and b, both of which span the membrane. Each chain has two domains. MHC class I molecules deliver peptides originating in the cytosol to the cell surface, where a peptide antigen-MHC (pMHC) complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a viral infection or the likelihood of recovery from the disease. In some embodiments, the use of statistical algorithms provides a prognosis of a viral infection in an individual. For example, the prognosis may be surgery, development of a clinical subtype of a viral infection, development of one or more clinical factors, or recovery from the disease.

As used herein, “percent identity” between amino acid sequences is synonymous with “percent homology,” which can be determined using the algorithm of Karlin and Altschul (1990) Proc. Natl. Acad. Sci. USA 87:2264-2268, modified by Karlin and Altschul (1993) Proc. Natl. Acad. Sci. USA 90:5873-5877. The noted algorithm is incorporated into the NBLAST and XBLAST programs of Altschul et al. (1990) J. Mol. Biol. 215:403-410. BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nuc. Acids Res. 25:3389-3402. When utilizing BLAST and Gapped BLAST programs, the default parameters of the respective programs (e.g., XBLAST and NBLAST) may be used.

The phrase “pharmaceutically-acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “ratio” refers to a relationship between two numbers (e.g., scores, summations, and the like). Although, ratios may be expressed in a particular order (e.g., a to b or a:b), one of ordinary skill in the art will recognize that the underlying relationship between the numbers may be expressed in any order without losing the significance of the underlying relationship, although observation and correlation of trends based on the ratio may be reversed.

The term “recombinant host cell” (or simply “host cell”) refers to a cell that comprises a nucleic acid that is not naturally present in the cell, such as a cell into which a recombinant expression vector has been introduced. It should be understood that cells according to the present invention is intended to refer not only to the particular subject cell, but also encompasses progeny of such a cell. Because certain modifications may occur in succeeding generations due to either mutation or environmental influences, such progeny may not, in fact, be identical to the parent cell, but are still included within the scope of the term cell according to the present invention.

The term “cancer response,” “response to immunotherapy,” or “response to modulators of T-cell mediated cytotoxicity/immunotherapy combination therapy” relates to any response of the hyperproliferative disorder (e.g., cancer) to a cancer agent, such as a modulator of T-cell mediated cytotoxicity, and an immunotherapy, preferably to a change in tumor mass and/or volume after initiation of neoadjuvant or adjuvant therapy. The term “neoadjuvant therapy” refers to a treatment given before the primary treatment. Examples of neoadjuvant therapy may include chemotherapy, radiation therapy, and hormone therapy. Hyperproliferative disorder response may be assessed, for example for efficacy or in a neoadjuvant or adjuvant situation, where the size of a tumor after systemic intervention may be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence. For example, in order to determine appropriate threshold values, a particular cancer therapeutic regimen may be administered to a population of subjects and the outcome may be correlated to biomarker measurements that were determined prior to administration of any cancer therapy. The outcome measurement may be pathologic response to therapy given in the neoadjuvant setting. Alternatively, outcome measures, such as overall survival and disease-free survival may be monitored over a period of time for subjects following cancer therapy for which biomarker measurement values are known. In certain embodiments, the doses administered are standard doses known in the art for cancer therapeutic agents. The period of time for which subjects are monitored may vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy may be determined using well-known methods in the art, such as those described in the Examples section.

As indicated, the terms may also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response may be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance may be mediated by P-glycoprotein or may be mediated by other mechanisms, or it may occur when a mammal is infected with a multi-drug-resistant microorganism or a combination of microorganisms. The determination of resistance to a therapeutic treatment is routine in the art and within the skill of an ordinarily skilled clinician, for example, may be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing logarithmically.

The term “sample” used for detecting or determining the absence, presence, or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a skin, colon sample, or surgical resection tissue. In some embodiments, methods encompassed by the present invention further comprises obtaining the sample from the individual prior to detecting or determining the absence, presence, or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa et al. (1982) Cancer Res. 42:2159-2164) and cell death assays (Weisenthal et al. (1984) Cancer Res. 94:161-173; Weisenthal et al. (1985) Cancer Treat Rep. 69:615-632; Weisenthal et al., In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal (1994) Contrib. Gynecol. Obstet. 19:82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy may be equally applied to methods for sensitizing hyperproliferative or otherwise cancerous cells (e.g., resistant cells) to the cancer therapy.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63-68), and natural product extract libraries. In another embodiment, the compounds are small, organic non-peptidic compounds. In a further embodiment, a small molecule is not biosynthetic.

The term “specific binding” refers to binding protein binding to a predetermined antigen. Typically, the binding protein binds with an affinity (K_{D}) of approximately less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or even lower, or any range in between, inclusive, such as between about 1-50 micromolar, 1-100 micromolar, 0.1-500 micromolar, and the like, when determined by a binding assay, such as surface plasmon resonance (SPR) technology in a BIAcore™ assay instrument using an antigen of interest as the analyte and the binding protein as the ligand. In some embodiments, the binding protein binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “a binding protein recognizing an antigen” and “a binding protein specific for an antigen” are used interchangeably herein with the term “a binding protein which binds specifically to an antigen.” Selective binding is a relative term referring to the ability of a binding protein to discriminate the binding of one antigen over another, such as a particular family member or antigen target over a related family member or antigen target. For example, analytical data provided in the Examples section demonstrates that binding proteins described herein specifically bind MAGEC2 immunogenic epitopes and/or selectively bind a number of related epitopes (e.g., MAGEC2 immunogenic epitopes and closely related sequences) discriminating such targets from the vast majority of other possible epitopes available in the human genome.

The term “subject” refers to any healthy animal, mammal or human, or any animal, mammal or human afflicted with a disorder characterized by MAGEC2 expression. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence.

The term “synergistic effect” refers to the combined effect of two or more agents (e.g., a MAGEC2-related agent described herein and another therapy for treating a disorder associated with MAGEC2 expression) that is greater than the sum of the separate effects of the cancer agents/therapies alone.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

A “T cell” is an immune system cell that matures in the thymus and produces T cell receptors (TCRs). T cells may be naive (not exposed to antigen; increased expression of CD62L, CCR7, CD28, CD3, CD 127, and CD45RA, and decreased expression of CD45RO as compared to T_{CM}), memory T cells (T_{M}) (antigen-experienced and long-lived), and effector cells (antigen-experienced, cytotoxic). T_{M} may be further divided into subsets of central memory T cells (T_{CM}, increased expression of CD62L, CCR7, CD28, CD127, CD45RO, and CD95, and decreased expression of CD54RA as compared to naive T cells) and effector memory T cells (T_{EM}, decreased expression of CD62L, CCR7, CD28, CD45RA, and increased expression of CD127 as compared to naive T cells or T_{CM}). Effector T cells (T_{E}) refers to antigen-experienced CD8+ cytotoxic T lymphocytes that have decreased expression of CD62L,CCR7, CD28, and are positive for granzyme and perforin as compared to T_{CM}. Other exemplary T cells include regulatory T cells, such as CD4⁺CD25⁺ (Foxp3⁺) regulatory T cells and Treg17 cells, as well as Tr1, Th3, CD8⁺CD28, and Qa-1 restricted T cells.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naive T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8⁺ T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). “T effector” (“T_{eff}” or “T_{E}”) cells refers to T cells (e.g., CD4+ and CD8+ T cells) with cytolytic activities as well as T helper (Th) cells, which secrete cytokines and activate and direct other immune cells, but does not include regulatory T cells (Treg cells).

“T cell receptor” or “TCR” refers to an immunoglobulin superfamily member (having a variable binding domain, a constant domain, a transmembrane region, and a short cytoplasmic tail; see, e.g., Janeway et al. (1997) Curr. Biol. Publ. 4:33) that is capable of binding (e.g., e.g., specifically and/or selectively) to an antigen peptide bound to an MHC receptor. A TCR can be found on the surface of a cell or in soluble form and generally is comprised of a heterodimer having alpha and beta chains (also known as TCRα and TCRβ, respectively), or γ and δ chains (also known as TCRγ and TCRδ, respectively). Like immunoglobulins (e.g., antibodies), the extracellular portion of TCR chains (e.g., α-chain and β-chain) contain two immunoglobulin domains: a variable domain (e.g., α-chain variable domain or V_{α} and β-chain variable domain or V_{β}; typically amino acids 1 to 116 based on Kabat numbering (Kabat et al. (1991) “Sequences of Proteins of Immunological Interest, US Dept Health and Human Services, Public Health Service National Institutes of Health, 5^{th} ed.) at the N-terminal end, and one constant domain (e.g., α-chain constant domain or Ca, typically amino acids 117 to 259 based on Kabat, β-chain constant domain or C_{β}, typically amino acids 117 to 295 based on Kabat) at the C-terminal end and adjacent to the cell membrane. Also like immunoglobulins, the variable domains contain complementary determining regions (“CDRs”, also called hypervariable regions or “HVRs”) separated by framework regions (“FRs”) (see, e.g., Fores et al. (1990) Proc. Natl. Acad Sci. US.A. 87:9138; Chothia et al. (1988) EMBO J. 7:3745; Lefranc et al. (2003) Dev. Comp. Immunol. 27:55). In some embodiments, a TCR is found on the surface of a T cell (or T lymphocyte) and associates with the CD3 complex. The source of a TCR encompassed by the present invention may be from various animal species, such as a human, mouse, rat, rabbit or other mammal.

The term “T cell receptor” or “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR may contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

Nomenclature established by the International Immunogenetics Information System (IMGT) (see also Scaviner and Lefranc (2000) Exp. Clin. Immunogenet. 17:83-96 and 97-106; Folch and Lefranc (2000) Exp. Clin. Immunogenet, 17:107-114; T Cell Receptor Factsbook”, (2001) LeFranc and LeFranc, Academic Press, ISBN 0-12-441352-8). The IMGT provides unique sequences used to describe a TCR, and sequences described herein may be identified by reference to such unique sequences provided herein. TCR sequences are publicly available at the IMGT database at imgt.org.

As described above, native alpha/beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three hypervariable CDRs (Complementarity Determining Regions) embedded in a framework sequence. CDR3 is well-known to be the main mediator of antigen recognition. There are several types of alpha chain variable (Vα) regions and several types of beta chain variable (Vβ) regions distinguished by their framework, CDR1 and CDR2 sequences, and by a partly defined CDR3 sequence. The Vα types are referred to in IMGT nomenclature by a unique TRAV number. For example, “TRAV4” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV2” defines a TCR VP region having unique framework and CDR1 and CDR2 sequences, but with only a partly defined CDR3 sequence. It is known that there are 54 alpha variable genes, of which 44 are functional, and 67 beta variable genes, of which 42 are functional, within the alpha and beta loci, respectively.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region.

The gene pools that encode the TCR alpha and beta chains are located on different chromosomes and contain separate V, (D), J and C gene segments, which are brought together by rearrangement during T cell development. This leads to a very high diversity of T cell alpha and beta chains due to the large number of potential recombination events that occur between the 54 TCR alpha variable genes and 61 alpha J genes or between the 67 beta variable genes, two beta D genes and 13 beta J genes. The recombination process is not precise and introduces further diversity within the CDR3 region. Each alpha and beta variable gene may also comprise allelic variants, designated in IMGT nomenclature as TRAVxx*01 and *02, or TRBVx-x*01 and *02 respectively, thus further increasing the amount of variation. In the same way, some of the TRBJ sequences have two known variations. (Note that the absence of a “*” qualifier means that only one allele is known for the relevant sequence). The natural repertoire of human TCRs resulting from recombination and thymic selection has been estimated to comprise approximately 106 unique beta chain sequences, determined from CDR3 diversity (Arstila et al. (1999) Science 286:958-961) and could be even higher (Robins et al. (2009) Blood 114:4099-4107). Each beta chain is estimated to pair with at least 25 different alpha chains, thus generating further diversity (Arstila et al. (1999) Science 286:958-961).

The term “TCR alpha variable domain” therefore refers to the concatenation of TRAV and TRAJ regions; a TRAV region only; or TRAV and a partial TRAJ region, and the term TCR alpha constant domain refers to the extracellular TRAC region, or to a C-terminal truncated or full length TRAC sequence. Likewise the term “TCR beta variable domain” refers to the concatenation of TRBV and TRBD/TRBJ regions; to the TRBV and TRBD regions only; to the TRBV and TRBJ regions only; or to the TRBV and partial TRBD and/or TRBJ regions, and the term TCR beta constant domain refers to the extracellular TRBC region, or to a C-terminal truncated or full length TRBC sequence. These TCR alpha variable domain and TCR beta variable domain nomenclature similarly applies to the variable domains of TCR gamma and TCR delta chains, respectively, for gamma/delta TCRs. An ordinarily skilled artisan can obtain TRAV, TRAJ, TRAC, TRBV, TRBJ, and TRBC gene sequences, such as through the publicly available IMGT database.

The term “TCR complex” refers to a complex formed by the association of CD3 with TCR For example, a TCR complex may be composed of a CD3γ chain, a CD3δ chain, two CD3ε chains, a homodimer of CD3ζ chains, a TCRα chain, and a TCRβ chain. Alternatively, a TCR complex may be composed of a CD3γ chain, a CD3δ chain, two CD3ε chains, a homodimer of CD3ζ chains, a TCRγ chain, and a TCRδ chain.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically effective amount” and “effective amount” means that amount of a substance that produces some desired effect, such as a desired local or systemic therapeutic effect, in at least a sub-population of cells in an animal at a reasonable benefit/risk ratio applicable to any treatment. In some embodiments, a therapeutically effective amount of a substance will depend on the substance's therapeutic index, solubility, pharmacokinetics, half-life, and the like. Toxicity and therapeutic efficacy of subject compounds may be determined by standard pharmaceutical procedures in cell cultures or experimental animals, e.g., for determining the LD₅₀ and the ED₅₀. In some embodiments, compositions that exhibit large therapeutic indices are used. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, T cell immune response in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a viral load may be achieved.

The term “treat” refers to the therapeutic management or improvement of a condition (e.g., a disease or disorder) of interest. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. A therapeutic agent may be administered to a subject who has a disease or is at increased risk of developing a disease relative to a member of the general population. In some embodiments, a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

The term “unresponsiveness” includes refractivity of cancer cells to therapy or refractivity of therapeutic cells, such as immune cells, to stimulation, e.g., stimulation via an activating receptor or a cytokine. Unresponsiveness may occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells may, however, proliferate if cultured with cytokines (e.g., IL-2). For example, T cell anergy may also be observed by the lack of IL-2 production by T lymphocytes as measured by ELISA or by a proliferation assay using an indicator cell line. Alternatively, a reporter gene construct may be used. For example, anergic T cells fail to initiate IL-2 gene transcription induced by a heterologous promoter under the control of the 5′ IL-2 gene enhancer or by a multimer of the AP1 sequence that may be found within the enhancer (Kang et al. (1992) Science 257:1134).

The term “vaccine” refers to a pharmaceutical composition that elicits an immune response to an antigen of interest. The vaccine may also confer protective immunity upon a subject.

The term “variable region” or “variable domain” refers to the domain of an immunoglobulin superfamily binding protein (e.g., a TCR α-chain or β-chain (or γ chain and δ chain for γδ TCRs)) that is involved in binding of the immunoglobulin superfamily binding protein (e.g., TCR) to antigen. The variable domains of the α-chain and β-chain (V_{α} and V_{β}, respectively) of a native TCR generally have similar structures, with each domain comprising four conserved framework regions (FRs) and three CDRs. The V_{α} domain is encoded by two separate DNA segments, the variable gene segment and the joining gene segment (V-J); the V_{β} domain is encoded by three separate DNA segments, the variable gene segment, the diversity gene segment, and the joining gene segment (V-D-J). A single V_{α} or V_{β} domain may be sufficient to confer antigen-binding specificity. Furthermore, TCRs that bind a particular antigen may be isolated using a V_{α} or V_{β} domain from a TCR that binds the antigen to screen a library of complementary V_{α} or V_{β} domains, respectively.

The term “vector” refers to a nucleic acid molecule capable of transporting another nucleic acid to which it has been linked. In some embodiments, a vector is an episome, i.e., a nucleic acid capable of extra-chromosomal replication. In some embodiments, vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the present invention is intended to include such other forms of expression vectors that serve equivalent functions and which become subsequently known in the art.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Peptides

In certain aspects, provided herein are methods and compositions for the treatment and/or prevention of disorders associated with MAGEC2 expression through the induction of an immune response against MAGEC2 or cells expressing MAGEC2 relating to administration of MAGEC2 immunogenic peptides, nucleic acids encoding same, and/or cells expressing same, described herein.

In certain embodiments, the MAGEC2 immunogenic peptide comprises (e.g., consists of) a peptide epitope selected from peptide sequences listed in Table 1, such as Table 1A and Table 1B. Peptide epitopes described herein may be combined with MHC molecules, such as particular HLA molecules having particular HLA alpha chain alleles. For example, Table 1A peptides were identified in association with an MHC whose alpha chain had an HLA-B*07 serotype, such as that encoded by an HLA-B*0702, HLA-B*0704, HLA-B*0705, HLA-B*0709, HLA-B*0710, HLA-B*0715, and/or HLA-B*0721 allele, as described further in the Examples section. Similarly, Table 1B peptides were identified in associated ion with an MHC whose alpha chain had an HLA-A*24 serotype, such as that encoded by an HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, as described further in the Examples section. In some embodiments, MAGEC2 immunogenic peptides may be combined with an MHC molecule, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In some embodiments, the MAGEC2 immunogenic peptides are derived from a human MAGEC2 protein and/or a MAGEC2 protein shown in Table 3. In some embodiments, one or more MAGEC2 immunogenic peptides are administered alone or in combination with an adjuvant.

In certain aspects, provided herein are compositions comprising one or more MAGEC2 immunogenic peptides described herein and an adjuvant.

TABLE 1
MAGEC2 epitopes
Table 1A
MAGEC2 epitopes presented by HLA serotype HLA-B*07
Peptide Epitopes

RAREFMEL
RAREFMELL
RAREFMELLF
LKRAREFMEL
VILKRAREF
FPVILKRAR
KRAREFMEL
KRAREFMELL
LKRAREFMELL
RAREFMELLFG

Table 1B
MAGEC2 epitopes presented by HLA serotype HLA-A *24
Peptide Epitopes

VGPDHFCVF
VGPDHFCVFA
VGPDHFCVFAN
VGPDHFCV
GPDHFCVF
EVGPDHFCVF
IEVGPDHFCVF

* Included in Table 1, such as Table 1A and Table 1B, are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequence listed in Table 1, such as Table 1A and Table 1B, or a portion thereof. Such polypeptides may have a function of the full-length peptide or polypeptide as described further herein.

In some embodiments, provided herein are MAGEC2 polypeptides and/or nucleic acids encoding MAGEC2 polypeptides. In some embodiments, MAGEC2 polypeptides are polypeptides that include an amino acid sequence of sufficient length to elicit a MAGEC2-specific immune response. In certain embodiments, the MAGEC2 polypeptide also includes amino acids that do not correspond to the amino acid sequence (e.g., a fusion protein comprising a MAGEC2 amino acid sequence and an amino acid sequence corresponding to a non-MAGEC2 protein or polypeptide). In some embodiments, the MAGEC2 polypeptide only includes amino acid sequence corresponding to a MAGEC2 protein or fragment thereof.

In some embodiments, the MAGEC2 polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 4445, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 373, or more, or any range in between inclusive (e.g., 7-25, 8-22, 9-22, etc.) consecutive amino acids of a MAGEC2 protein amino acid sequence, such as those set forth in Table 3. In some embodiments, the consecutive amino acids are identical to an amino acid sequence of MAGEC2 set forth in Table 3. In some embodiments, MAGEC2 polypeptides comprise, consist essentially of, or consist of one or more peptide epitopes selected from the group consisting of MAGEC2 peptide epitopes listed in Table 1, such as Table 1A and Table 1B.

As is well-known to those skilled in the art, polypeptides having substantial sequence similarities can cause identical or very similar immune reaction in a host animal. Accordingly, in some embodiments, a derivative, equivalent, variant, fragment, or mutant of a MAGEC2 immunogenic peptide described herein or fragment thereof may also suitable for the methods and compositions provided herein.

In some embodiments, variations or derivatives of the MAGEC2 immunogenic polypeptides are provided herein. The altered polypeptide may have an altered amino acid sequence, for example by conservative substitution, yet still elicits immune responses which react with the unaltered protein antigen, and are considered functional equivalents. As used herein, the term “conservative substitution” denotes the replacement of an amino acid residue by another, biologically similar residue. It is well-known in the art that the amino acids within the same conservative group may typically substitute for one another without substantially affecting the function of a protein. According to certain embodiments, the derivative, equivalents, variants, or mutants of the ligand-binding domain of a MAGEC2 immunogenic peptide are polypeptides that are at least 85% homologous to the sequence of a MAGEC2 immunogenic peptide described herein or fragment thereof. In some embodiments, the homology is at least 90%, at least 95%, at least 98%, or more.

Immunogenic peptides encompassed by the present invention may comprise a peptide epitope derived from a MAGEC2 protein, such as those listed in Table 1, such as Table 1A and Table 1B. In some embodiments, the immunogenic peptide is 8, 9, 10, 11, 12, 13, 14, or 15 amino acids in length. In some embodiments, the peptide amino acid sequences is modified, which may include conservative or non-conservative mutations. A peptide may comprise at most 1, 2, 3, 4, or more mutations. In some embodiments, a peptide may comprise at least 1, 2, 3, 4, or more mutations.

In some embodiments, a peptide may be chemically modified. For example, a peptide can be mutated to modify peptide properties such as detectability, stability, biodistribution, pharmacokinetics, half-life, surface charge, hydrophobicity, conjugation sites, pH, function, and the like. N-methylation is one example of methylation that can occur in a peptide of the disclosure. In some embodiments, a peptide may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region may be a fusion Fc-peptide. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing. In some embodiments, the peptides encompassed by the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide encompassed by the present disclosure. In other embodiments, a peptide may include post-translational modifications (e.g., methylation and/or amidation), which can affect, for example, serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the peptides may be conjugated to myristic acid (tetradecanoic acid) or a derivative thereof. In other embodiments, a peptide may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a peptide, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, fusion proteins or peptides may be conjugated to other moieties that, for example, can modify or effect changes to the properties of the peptides.

A peptide may, in some embodiments, be covalently linked to a moiety. In some embodiments, the covalently linked moiety comprises an affinity tag or a label. The affinity tag may be selected from the group consisting of glutathione-S-transferase (GST), calmodulin binding protein (CBP), protein C tag, Myc tag, HaloTag, HA tag, Flag® tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A peptide may be conjugated to an agent used in imaging, research, therapeutics, theranostics, pharmaceuticals, chemotherapy, chelation therapy, targeted drug delivery, and radiotherapy. In some embodiments, a peptide may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that may be used as a conjugating molecule include DyLight®-680, DyLight®-750, VivoTag®-750, DyLight®-800, IRDye®-800, VivoTag®-680, Cy5.5, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors® (e.g., Alexa Fluor® 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR™ and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green® Dyes (e.g., Oregon Green® 488, Oregon Green® 500, Oregon Green® 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR® dyes (e.g., ALEXA FLUOR® 350, ALEXA FLUOR® 488, ALEXA FLUOR® 532, ALEXA FLUOR® 546, ALEXA FLUOR® 568, ALEXA FLUOR® 594, ALEXA FLUOR® 633, ALEXA FLUOR® 660, ALEXA FLUOR® 680, etc.), BODIPY® dyes (e.g., BODIPY® FL, BODIPY® R6G, BODIPY® TMR, BODIPY® TR, BODIPY® 530/550, BODIPY® 558/568, BODIPY® 564/570, BODIPY® 576/589, BODIPY® 581/591, BODIPY® 630/650, BODIPY® 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

A peptide may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the peptide is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain pMHC binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, oc-aminocyclohexane carboxylic acid, α-aminocycloheptane carboxylic acid, α-(2-amino-2-norbomane)-carboxylic acid, α,γ-diaminobutyric acid, β-diaminopropionic acid, homophenylalanine, and oc-tert-butylglycine.

Binding proteins encompassed by the present invention may be glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A protein such as a peptide may be produced recombinantly or synthetically, such as by solid-phase peptide synthesis or solution-phase peptide synthesis. Protein synthesis may be performed by known synthetic methods, such as using fluorenylmethyloxycarbonyl (Fmoc) chemistry or by butyloxycarbonyl (Boc) chemistry. Protein fragments may be joined together enzymatically or synthetically.

In an aspect encompassed by the present invention, provided herein are methods of producing a protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

In some embodiments, provided herein is a nucleic acid encoding a MAGEC2 immunogenic peptide described herein or fragment thereof, such as a DNA molecule encoding a MAGEC2 immunogenic peptide. In some embodiments, the composition comprises an expression vector comprising an open reading frame encoding a MAGEC2 immunogenic peptide described herein or fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the MAGEC2 immunogenic polypeptide or fragment thereof. Representative vectors, promoters, regulatory elements, and the like useful for expressing proteins such as peptide are described further below.

### III. MHC-Peptide Complexes

In certain aspects, provided herein are compositions comprising a MAGEC2 immunogenic peptide described herein and a MHC molecule. In some embodiments, the MAGEC2 immunogenic peptide forms a stable complex with the MHC molecule.

MHC proteins may be conjugated to an agent, such as a detection moiety, readiosensitizer, photosensitizer, and the like, and/or may be chemically modified as described above regarding peptides.

The MHC proteins provided and used in the compositions and methods encompassed by the present disclosure may be any suitable MHC molecules known in the art. Generally, they have the formula (α-β-P)ₙ, where n is at least 2, for example between 2-10, e.g., 4. α is an α chain of a class I or class II MHC protein. β is a β chain, herein defined as the β chain of a class II MHC protein or β₂ microglobulin for a MHC class I protein. P is a peptide antigen.

In some embodiments, the MHC proteins are MHC class I complexes, such as HLA I complexes.

The MHC proteins may be from any mammalian or avian species, e.g., primate sp., particularly humans; rodents, including mice, rats and hamsters; rabbits; equines, bovines, canines, felines; etc. For instance, the MHC protein may be derived the human HLA proteins or the murine H-2 proteins. HLA proteins include the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. H-2 proteins include the class I subunits H-2K, H-2D, H-2L, and the class II subunits I-Aα, I-Aβ, I-Eα and I-Eβ, and β2-microglobulin. Sequences of some representative MHC proteins may be found in Kabat et al. Sequences of Proteins of Immunological Interest, NIH Publication No. 91-3242, pp 724-815. MHC protein subunits suitable for use in the present invention are a soluble form of the normally membrane-bound protein, which is prepared as known in the art, for instance by deletion of the transmembrane domain and the cytoplasmic domain.

For class I proteins, the soluble form may include the α1, α2 and α3 domain. Soluble class II subunits may include the α1 and α2 domains for the α subunit, and the β1 and β2 domains for the β subunit.

The α and β subunits may be separately produced and allowed to associate in vitro to form a stable heteroduplex complex, or both of the subunits may be expressed in a single cell. Methods for producing MHC subunits are known in the art.

In certain embodiments, the MHC-peptide complex comprises a peptide epitope selected from Table 1 (such as Table 1A and Table 1B) and an MHC. In some embodiments, the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In some embodiments, the MHC-peptide complex comprises a peptide epitope selected from Table 1A and an MHC whose alpha chain has an HLA-B*07 serotype, such as that encoded by an HLA-B*0702, HLA-B*0704, HLA-B*0705, HLA-B*0709, HLA-B*0710, HLA-B*0715, and/or HLA-B*0721 allele. In some embodiments, the MHC-peptide complex comprises a peptide epitope selected from Table 1B and an MHC whose alpha chain has an HLA-A*24 serotype, such as that encoded by an HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, and/or HLA-A*2458 allele.

To prepare the MHC-peptide complex, the subunits may be combined with an antigenic peptide and allowed to fold in vitro to form a stable heterodimer complex with intrachain disulfide bonded domains. The peptide may be included in the initial folding reaction, or may be added to the empty heterodimer in a later step. In the compositions and methods encompassed by the present invention, this is a MAGEC2 immunogenic peptide or fragment thereof. Conditions that permit folding and association of the subunits and peptide are known in the art. As one example, roughly equimolar amounts of solubilized a and β subunits may be mixed in a solution of urea. Refolding is initiated by dilution or dialysis into a buffered solution without urea. Peptides may be loaded into empty class II heterodimers at about pH 5 to 5.5 for about 1 to 3 days, followed by neutralization, concentration and buffer exchange. However, the specific folding conditions are not critical for the practice of the invention.

The monomeric complex (α-β-P) (herein monomer) may be multimerized, for example, for a MHC tetramer. The resulting multimer is stable over long periods of time. Preferably, the multimer may be formed by binding the monomers to a multivalent entity through specific attachment sites on the α or β subunit, as known in the art (e.g., as described in U.S. Pat. No. 5,635,363). The MHC proteins, in either their monomeric or multimeric forms, may also be conjugated to beads or any other support.

The multimeric complex may be labeled, so as to be directly detectable when used in immunostaining or other methods known in the art, or may be used in conjunction with secondary labeled immunoreagents which specifically and/or selectively bind the complex (e.g., bind to a MHC protein subunit) as known in the art. For example, the detectable label may be a fluorophore, such as fluorescein isothiocyanate (FITC), rhodamine, Texas Red, phycoerythrin (PE), allophycocyanin (APC), Brilliant Violet™ 421, Brilliant UV™ 395, Brilliant Violet™ 480, Brilliant Violet™ 421 (BV421), Brilliant Blue™ 515, APC-R700, or APC-Fire750. In some embodiments, the multimeric complex is labeled by a moiety that is capable of specifically and/or selectively binding another moiety. For instance, the label may be biotin, streptavidin, an oligonucleotide, or a ligand. Other labels of interest may include fluorochromes, dyes, enzymes, chemiluminescers, particles, radioisotopes, or other directly or indirectly detectable agent.

In some embodiments, a cell presenting an immunogenic peptides in context of an MHC molecule on the cell surface is generated by transfecting or transducing the cell with a vector (e.g., a viral vector) that comprising nucleic acid that encodes a recombinant or heterologous antigen into a cell. In some embodiments, the vector is introduced into the cell under conditions in which one or more peptide antigens, including, in some cases, one or more peptide antigens of the expressed heterologous protein, are expressed by the cell, processed and presented on the surface of the cell in the context of a major histocompatibility complex (MHC) molecule.

Generally, the cell to which the vector is contacted is a cell that expresses MHC, i.e., MHC-expressing cells. The cell may be one that normally expresses an MHC on the cell surface, that is induced to express and/or upregulate expression of MHC on the cell surface or that is engineered to express an MHC molecule on the cell surface. In some embodiments, the MHC contains a polymorphic peptide binding site or binding groove that can, in some cases, complex with peptide antigens of polypeptides, including peptide antigens processed by the cell machinery. In some cases, MHC molecules may be displayed or expressed on the cell surface, including as a complex with peptide, i.e., MHC-peptide complex, for presentation of an antigen in a conformation recognizable by TCRs on T cells, or other peptide binding molecules.

In some embodiments, the cell is a nucleated cell. In some embodiments, the cell is an antigen-presenting cell. In some embodiments, the cell is a macrophage, dendritic cell, B cell, endothelial cell or fibroblast. In some embodiments, the cell is an endothelial cell, such as an endothelial cell line or primary endothelial cell. In some embodiments, the cell is a fibroblast, such as a fibroblast cell line or a primary fibroblast cell.

In some embodiments, the cell is an artificial antigen presenting cell (aAPC). Typically, aAPCs include features of natural APCs, including expression of an MHC molecule, stimulatory and costimulatory molecule(s), Fc receptor, adhesion molecule(s) and/or the ability to produce or secrete cytokines (e.g., IL-2). Normally, an aAPC is a cell line that lacks expression of one or more of the above, and is generated by introduction (e.g., by transfection or transduction) of one or more of the missing elements from among an MHC molecule, a low affinity Fc receptor (CD32), a high affinity Fc receptor (CD64), one or more of a co-stimulatory signal (e.g., CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, HVEM, lymphotoxin beta receptor, ILT3, ILT4, 3/TR6 or a ligand of B7-H3; or an antibody that specifically binds to CD27, CD28, 4-1BB, OX40, CD30, CD40, PD-1, ICOS, LFA-1, CD2, CD7, LIGHT, NKG2C, B7-H3, Toll ligand receptor or a ligand of CD83), a cell adhesion molecule (e.g., ICAM-1 or LFA-3) and/or a cytokine (e.g., IL-2, IL-4, IL-6, IL-7, IL-10, IL-12, IL-15, IL-21, interferon-alpha (IFN.alpha.), interferon-beta (IFN.beta.), interferon-gamma (IFN.gamma.), tumor necrosis factor-alpha (TNF.alpha.), tumor necrosis factor-beta (TNF.beta.), granulocyte macrophage colony stimulating factor (GM-CSF), and granulocyte colony stimulating factor (GCSF)). In some cases, an aAPC does not normally express an MHC molecule, but may be engineered to express an MHC molecule or, in some cases, is or may be induced to express an MHC molecule, such as by stimulation with cytokines. In some cases, aAPCs also may be loaded with a stimulatory ligand, which may include, for example, an anti-CD3 antibody, an anti-CD28 antibody or an anti-CD2 antibody. An exemplary cell line that may be used as a backbone for generating an aAPC is a K562 cell line or a fibroblast cell line. Various aAPCs are known in the art, see e.g., U.S. Pat. No. 8,722,400, published application No. US2014/0212446; Butler and Hirano (2014) Immunol Rev. 257:10. 1111/imr.12129; Suhoshki et al. (2007) Mol. Ther. 15:981-988).

It is well within the level of a skilled artisan to determine or identify the particular MHC or allele expressed by a cell. In some embodiments, prior to contacting cells with a vector, expression of a particular MHC molecule may be assessed or confirmed, such as by using an antibody specific for the particular MHC molecule. Antibodies to MHC molecules are known in the art, such as any described below.

In some embodiments, the cells may be chosen to express an MHC allele of a desired MHC restriction. In some embodiments, the MHC typing of cells, such as cell lines, are well-known in the art. In some embodiments, the MHC typing of cells, such as primary cells obtained from a subject, may be determined using procedures well-known in the art, such as by performing tissue typing using molecular haplotype assays (BioTest ABC SSPtray, BioTest Diagnostics Corp., Denville, N.J.; SeCore Kits, Life Technologies, Grand Island, N.Y.). In some cases, it is well within the level of a skilled artisan to perform standard typing of cells to determine the HLA genotype, such as by using sequence-based typing (SBT) (Adams et al. (2004) J. Transl. Med., 2:30; Smith (2012) Methods Mol Biol., 882:67-86). In some cases, the HLA typing of cells, such as fibroblast cells, are known. For example, the human fetal lung fibroblast cell line MRC-5 is HLA-A*0201, A29, B13, B44 Cw7 (C*0702); the human foreskin fibroblast cell line Hs68 is HLA-A1, A29, B8, B44, Cw7, Cw16; and the WI-38 cell line is A*6801, B*0801, (Solache et al. (1999) J Immunol, 163:5512-5518; Ameres et al. (2013) PloS Pathog. 9:e1003383). The human transfectant fibroblast cell line M1DR1/Ii/DM express HLA-DR and HLA-DM (Karakikes et al. (2012) FASEB J., 26:4886-96).

In some embodiments, the cells to which the vector is contacted or introduced are cells that are engineered or transfected to express an MHC molecule. In some embodiments, cell lines may be prepared by genetically modifying a parental cells line. In some embodiments, the cells are normally deficient in the particular MHC molecule and are engineered to express such particular MHC molecule. In some embodiments, the cells are genetically engineered using recombinant DNA techniques.

In some embodiments, the stable MHC-peptide complexes described herein are used to detect T cells that bind a stable MHC-peptide complex. In some embodiments, the stable MHC-peptide complexes described herein are used to monitor T cell response in a subject, for example, by detecting the amount and/or percentage of T cells (e.g., CD8+ T cells) that specifically and/or selectively bind to the MHC-peptide complexes that are fluorescently labeled. Methods of generating, labeling, and using MHC-peptide complexes (e.g., MHC-peptide tetramers) for detecting MHC-peptide complex-specific T cells are well-known in the art. Additional description can be found in, for example, U.S. Pat. Nos. 7,776,562; 8,268,964; and U.S. Pat. Publ. 2019/0085048.

### IV. Immunogenic Compositions

In some aspects, provided herein are pharmaceutical compositions (e.g., a vaccine composition) comprising a MAGEC2 immunogenic peptide and/or a nucleic acid encoding a MAGEC2 immunogenic peptide and an adjuvant. In some aspects, provided herein are pharmaceutical compositions (e.g., a vaccine composition) comprising a stable MHC-peptide complex comprising a MAGEC2 immunogenic peptide in the context of a MHC molecule and an adjuvant. In some embodiments, the composition includes a combination of multiple (e.g., two or more) MAGEC2 immunogenic peptides or nucleic acids and an adjuvant. In some embodiments, the composition includes a combination of multiple (e.g., two or more) stable MHC-peptide complexes comprising a MAGEC2 immunogenic peptide in the context of a MHC molecule and an adjuvant. In some embodiments, the compositions described above further comprises a pharmaceutically acceptable carrier.

The pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association a MAGEC2 immunogenic peptide and/or nucleic acid described herein with the adjuvant, carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise MAGEC2 immunogenic peptides and/or nucleic acids described herein in combination with a adjuvant, as well as one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

In some embodiments, the pharmaceutical composition described, when administered to a subject, can elicit an immune response against a cell that is infected by MAGEC2. Such pharmaceutical compositions may be useful as vaccine compositions for prophylactic and/or therapeutic treatment of disorders characterized by MAGEC2 expression.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition according to the invention after reconstitution of the peptides and optional emulsification with an oil-based adjuvant as defined above, (ii) may be part of the reconstitution composition of the invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens (i.e., antigens present in the MAGEC2 immunogenic peptide). For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. In some embodiments, the adjuvant is Adjuvant 65, α-GalCer, aluminum phosphate, aluminum hydroxide, calcium phosphate, β-Glucan Peptide, CpG DNA, GM-CSF, GPI-0100, IFA, IFN-γ, IL-17, lipid A, lipopolysaccharide, Lipovant, Montanide, N-acetyl-muramyl-L-alanyl-D-isoglutamine, Pam3CSK4, quil A, trehalose dimycolate or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-L.alpha., MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, I309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a nucleic acid encoding an MAGEC2 immunogenic polypeptide described herein, such as a DNA molecule encoding a MAGEC2 immunogenic peptide. In some embodiments the composition comprises an expression vector comprising an open reading frame encoding a MAGEC2 immunogenic peptide.

When taken up by a cell (e.g., host cell, an antigen-presenting cell (APC) such as a dendritic cell, macrophage, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### V. Binding Proteins

In some aspects, a binding moiety that binds a peptide described herein and/or a stable MHC-peptide complex described herein, are provided. For example, binding proteins like T cell receptors (TCRs), antibodies, and the like that specifically and/or selectively bind to the peptide and/or the stable MHC-peptide complex, such as with a K_{d} less than or equal to about 10⁻⁴ M (e.g., about 10⁻⁴, 10⁻⁵, 10⁻⁶, 10⁻⁷, about 10⁻⁸, about 10⁻⁹, about 10¹⁰, about 10⁻¹¹, about 10⁻¹², about 10⁻¹³, about 10⁻¹⁴, etc.), are provided.

In an aspect encompassed by the present invention, provided herein are binding proteins that bind (e.g., specifically and/or selectively) to a peptide-MHC (pMHC) complex comprising a MAGEC2 immunogenic peptide in the context of an MHC molecule (e.g., an MHC class I molecule). In some embodiments, the binding protein is capable of binding (e.g., specifically and/or selectively) to a MAGEC2 peptide-MHC (pMHC) complex with a K_{d} less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or any range in between, inclusive, such as between about 1-50 micromolar, 1-100 micromolar, 0.1-500 micromolar, and the like. In some embodiments, the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In some embodiments, the HLA serotype is HLA-B*07 and/or the HLA allele is selected from the group consisting of HLA-B*0702, HLA-B*0704, HLA-B*0705, HLA-B*0709, HLA-B*0710, HLA-B*0715, and HLA-B*0721 alleles. In a specific embodiment, the HLA allele is HLA-B*0702. In some embodiments, the HLA serotype is HLA-A*24 and/or the HLA allele is selected from the group consisting of HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, and HLA-A*2458 alleles. In a specific embodiment, the HLA allele is HLA-A*2402. In some embodiments, the binding proteins provided herein are genetically engineered, isolated, and/or purified.

In some embodiments, the binding proteins have a higher binding affinity to the MAGEC2 peptide-MHC (pMHC) than does a known T-cell receptor (e.g., a TCR from van Kunert et al. (2016) J. Immunol. 197:2541-2552 or others described herein). For example, the binding proteins may have at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, 5000 fold, 10000 fold, 50000 fold, 100000 fold, 500000 fold, 1000000 fold, or more, or any range in between, inclusive, such as 1.2 fold to 2 fold, higher binding affinity to the MAGEC2 peptide-MHC (pMHC) than does a known T-cell receptor.

In some embodiments, the binding protein induces higher T cell expansion, cytokine release, and/or cytotoxic killing than does a known T-cell receptor when contacted with target cells with expression of MAGEC2 at a certain level or below (e.g., see the Examples section for representative cell lines expressing MAGEC2 at varying levels). For example, in some embodiments of any aspect described herein, MAGEC2 level can be expressed in terms of transcripts per million and may be, for example, less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 73 TPM). As described further herein, TPM is measured according to well-known techniques, such as RNA-Seq, and gene expression TPM data are well-known in the art for a variety of cell lines, tissue types, and the like (see, for example, the Broad Institute Cancer Cell Line Encyclopedia (CCLE) on the World Wide Web at portals.broadinstitute.org). In some embodiment, the binding protein induces at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, increase in T cell expansion, cytokine release, and/or cytotoxic killing than does a known T-cell receptor when contacted with target cells with heterozygous expression of MAGEC2.

In some embodiments, the expression of MAGEC2 is detected using RNA-sequencing (RNA-seq). RNA-seq generally comprises the following steps: obtaining a sample containing genetic material, isolating total RNA from the sample obtained, preparing an amplified cDNA library from the total RNA, sequencing the amplified cDNA library, and analyzing and profiling the amplified cDNA to assess the expression level of different transcripts. The sample can be a population of cells, a tissue sample, a biopsy sample, a cell culture, or a single cell. Total RNA can be isolated from the biological sample using any method known in the art. In certain embodiments, total RNA is extracted from plasma. Plasma RNA extraction is described in Enders et al., “The Concentration of Circulating Corticotropin-Releasing Homer mRNA in Material Plasma Is Inclined in Preeclampsia,” Clinr. As described therein, the plasma collected after the centrifugation step is mixed with Trizol LS reagent (Invitrogen) and chloroform. The mixture is centrifuged and the aqueous layer is transferred to a new tube. Ethanol is added to this aqueous layer. The mixture is then placed in an RNeasy mini column (Qiagen) and processed according to the manufacturer's recommendations.

In some embodiments, RNA-seq described herein includes the step of preparing amplified cDNA from total RNA. For example, cDNA is prepared and the isolated RNA sample is randomly amplified without dilution, or the mixture of genetic material in the isolated RNA is dispersed into individual reaction samples. In certain embodiments, amplification is initiated randomly at the 3′end and throughout the entire transcriptome in the sample to amplify both mRNA and non-polyadenylated transcripts. In this way, double-stranded cDNA amplification products are optimized for the generation of sequencing libraries for next generation sequencing platforms. A kit suitable for amplification of cDNA by the method encompassed by the present invention includes, for example, Ovation® RNA-Seq System.

In some embodiments, RNA-seq described herein includes the step of sequencing the amplified cDNA. Any known sequencing method can be used to sequence the amplified cDNA mixture including the single molecule sequencing method. In certain embodiments, the amplified cDNA is sequenced by whole transcriptome shotgun sequencing. Whole transcriptome shotgun sequencing can be performed using various next generation sequencing platforms such as Illumina® Genome Analyzer platform, ABI SOLiD™ Sequencing platform, or Life Science's 454 Sequencing platform.

In some embodiments, RNA-seq described herein further comprises performing digital counting and analysis on the cDNA. The number of amplified sequences for each transcript in the amplified sample can be quantified by sequence reading (one reading per amplified strand). In some embodiments, transcript per million (TPM) is used to quantify the expression level of a particular transcript. TPM may be calculated as shown in Wagner et al. (2012) Theory in Biosciences 131:281-285, the content of which is incorporated by reference herein in its entirety.

In certain embodiments, the binding proteins recognize a MAGEC2 immunogenic peptide in a complex with MHC molecules, such as particular HLA molecules having particular HLA alpha chain alleles. For example, binding proteins listed in Table 2A were identified as binders of MAGEC2 immunogenic peptides in association with an MHC whose alpha chain had an HLA-B*07 serotype, such as that encoded by an HLA-B*0702, HLA-B*0704, HLA-B*0705, HLA-B*0709, HLA-B*0710, HLA-B*0715, and/or HLA-B*0721 allele, as described further in the Examples section. Similarly, binding proteins listed in Table 2B were identified as binders of MAGEC2 immunogenic peptides in association with an MHC whose alpha chain had an HLA-A*24 serotype, such as that encoded by an HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, and/or HLA-A*2458 allele, as described further in the Examples section. In some embodiments, the binding proteins recognize a complex of MAGEC2 immunogenic peptide and an MHC molecule, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In some embodiments, the MAGEC2 immunogenic peptides are derived from a human MAGEC2 protein and/or a MAGEC2 protein shown in Table 3. In some embodiments, one or more MAGEC2 immunogenic peptides are administered alone or in combination with an adjuvant.

In some embodiments, the binding proteins provided herein include (e.g., comprise, consist essentially of, or consist of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in Table 2; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in Table 2.

In some embodiments, the binding proteins provided herein include (e.g., comprise, consist essentially of, or consist of): a) a TCR alpha chain sequence selected from the group consisting of the TCR alpha chain sequences listed in Table 2; and/or b) a TCR beta chain sequence selected from the group consisting of the TCR beta chain sequences listed in Table 2.

In some embodiments, the binding proteins provided herein include (e.g., comprise, consist essentially of, or consist of): a) a TCR alpha chain variable (V) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in Table 2; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR V_{β} domain sequences listed in Table 2.

In some embodiments, the binding proteins provided herein include (e.g., comprise, consist essentially of, or consist of): a) a TCR alpha chain variable (V_{α}) domain sequence selected from the group consisting of the TCR V_{α} domain sequences listed in Table 2; and/or b) a TCR beta chain variable (Vβ) domain sequence selected from the group consisting of the TCR V_{β} domain sequences listed in Table 2.

In some embodiments, the binding proteins provided herein include (e.g., comprise, consist essentially of, or consist of at least one (e.g., one, two or three, such as CDR3 alone or in combination with a CDR1 and CDR2)) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95% 96%, 97%, 98%, 99%, or more identity to a TCR alpha chain CDR sequence selected from the group consisting of the TCR alpha chain CDR sequences listed in Table 2. CDR3 is believed to be the main CDR responsible for recognizing processed antigen and CDR1 and CDR2 mainly interact with the MHC, so, in some embodiments, binding protein comprising a CDR3 alone from a TCR alpha chain and/or a CDR3 alone from a TCR beta chain listed in Table 2, each CDR3 having a sequence homology as recited in this paragraph, are provided.

In some embodiments, the binding proteins provided herein may also include (e.g., comprise, consist essentially of, or consist of at least one (e.g., one, two or three, such as CDR3 alone or in combination with a CDR1 and CDR2)) TCR beta chain complementarity determining region (CDR) sequence with at least about 80%, 81%, 82%, 83%, 84%, 85%, 86%, 87%, 88%, 89%, 90%, 91%, 92%, 93%, 94%, 95% 96%, 97%, 98%, 99%, or more identity to a TCR beta chain CDR sequence selected from the group consisting of the TCR beta chain CDR sequences listed in Table 2. As described above, CDR3 is believed to be the main CDR responsible for recognizing processed antigen and CDR1 and CDR2 mainly interact with the MHC, so, in some embodiments, binding protein comprising a CDR3 alone from a TCR beta chain and/or a CDR3 alone from a TCR alpha chain listed in Table 2, each CDR3 having a sequence homology as recited in this paragraph, are provided.

In some embodiments, the binding proteins provided herein include (e.g., comprise, consist essentially of, or consist of at least one (e.g., one, two or three)) TCR alpha chain complementarity determining region (CDR) listed in Table 2.

In some embodiments, the binding proteins provided herein may also include (e.g., comprise, consist essentially of, or consist of at least one (e.g., one, two or three)) TCR beta chain complementarity determining region (CDR) listed in Table 2.

In some embodiments, the binding proteins provided herein include (e.g., comprise, consist essentially of, or consist of) a TCR alpha chain constant region (C_{α}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR Ca sequence listed in Table 2.

In some embodiments, the binding proteins provided herein may also include (e.g., comprise, consist essentially of, or consist of) a TCR beta chain constant region (C_{β}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{β} sequence listed in Table 2.

In some embodiments, the binding proteins provided herein include (e.g., comprise, consist essentially of, or consist of) a TCR alpha chain constant region (C_{α}) sequence selected from the group consisting of the TCR C_{α} sequences listed in Table 2.

In some embodiments, the binding proteins provided herein may also include (e.g., comprise, consist essentially of, or consist of) a TCR beta chain constant region (C_{β}) sequence selected from the group consisting of the TCR C_{β} sequences listed in Table 2.

TABLE 2
TCR sequences recognizing a MAGEC2 antigen
Table 2A
TCR sequences recognizing a MAGEC2 antigen presented by
HLA serotype HLA-B*07
MAGEC2 TCR 8-3 wild type sequence
Alpha chain:
TRAV24*01 F/TRAJ32*02/TRAC
Alpha chain DNA sequence
ATGGAGAAGAATCCTTTGGCAGCCCCACTTCTTATCCTCTGGTTTCATCTTGACTGCGTGAGCAGC
ATTCTGAACGTGGAACAAAGTCCTCAGTCACTGCATGTTCAGGAGGGAGACAGCACCAATTTCACC
TGCAGCTTCCCTTCCAGCAATTTTTATGCCCTTCACTGGTACAGATGGGAAACTGCAAAAAGCCCC
GAGGCCTTGTTTGTTATGACTCTTAATGGGGATGAAAAGAAGAAAGGACGCATTAGTGCCACTCTT
AATACCAAGGAGGGTTACAGCTATTTGTATATCAAAGGATCCCAGCCTGAGGACTCAGCCACATAC
CTCTGTGCCTCCGGAAGTGGTGGTGCTACAAACAAGCTCATCTTTGGAACTGGCACTCTGCTTGCT
GTCCAGCCAAatatccagaaccctgaccctgccgtgtaccagctgagagactctaaatccagtgac
aagtctgtctgcctattcaccgattttgattctcaaacaaatgtgtcacaaagtaaggattctgat
gtgtatatcacagacaaaactgtgctagacatgaggtctatggacttcaagagcaacagtgctgtg
gcctggagcaacaaatctgactttgcatgtgcaaacgccttcaacaacagcattattccagaagac
accttcttccccagcccagaaagttcctgtgatgtcaagctggtcgagaaaagctttgaaacagat
acgaacctaaactttcaaaacctgtcagtgattgggttccgaatcctcctcctgaaagtggccggg
tttaatctgctcatgacgctgcggctgtggtccagc
alpha chain protein sequence
MEKNPLAAPLLILWFHLDCVSSILNVEQSPQSLHVQEGDSTNFTCSFPSSNFYALHWYRWETAKSP
EALFVMTLNGDEKKKGRISATLNTKEGYSYLYIKGSQPEDSATYLCASGSGGATNKLIFGTGTLLA
VQPniqnpdpavyqlrdskssdksvclftdfdsqtnvsqskdsdvyitdktvldmrsmdfksnsav
awsnksdfacanafnnsiipedtffpspesscdvklveksfetdtnInfqnlsvigfrilllkvag
fnllmtlrlwss
Beta chain:
TRBV16*01/TRBJ1-1*01/TRBC1
Beta chain DNA sequence
ATGAGCCCAATTTTCACCTGCATCACAATCCTTTGTCTGCTGGCTGCAGGTTCTCCTGGTGAAGAA
GTCGCCCAGACTCCAAAACATCTTGTCAGAGGGGAAGGACAGAAAGCAAAACTTTATTGTGCCCCA
ATTAAAGGACACAGTTATGTTTTCTGGTACCAACAGGTCCTGAAAAACGAGTTCAAGTTCTTGATT
TCCTTCCAGAATGAAAATGTCTTTGATGAAACAGGTATGCCCAAGGAAAGATTTTCAGCTAAGTGC
CTCCCAAATTCACCCTGTAGCCTTGAGATCCAGGCTACTAAGCTTGAGGATTCAGCAGTGTATTTT
TGTGCCAGCAGCCAATCACGGAGCCTTAGGGGCACTGAAGCTTTCTTTGGACAAGGCACCAGACTC
ACAGTTGTTGaggacctgaacaaggtgttcccacccgaggtcgctgtgtttgagccatcagaagca
gagatctcccacacccaaaaggccacactggtgtgcctggccacaggcttcttccctgaccacgtg
gagctgagctggtgggtgaatgggaaggaggtgcacagtggggtcagcacggacccgcagcccctc
aaggagcagcccgccctcaatgactccagatactgcctgagcagccgcctgagggtctcggccacc
ttctggcagaacccccgcaaccacttccgctgtcaagtccagttctacgggctctcggagaatgac
gagtggacccaggatagggccaaacccgtcacccagatcgtcagcgccgaggcctggggtagagca
gactgtggctttacctcggtgtcctaccagcaaggggtcctgtctgccaccatcctctatgagatc
ctgctagggaaggccaccctgtatgctgtgctggtcagcgcccttgtgttgatggccatggtcaag
agaaaggatttc
Beta chain protein sequence
MSPIFTCITILCLLAAGSPGEEVAQTPKHLVRGEGQKAKLYCAPIKGHSYVFWYQQVLKNEFKFLI
SFQNENVFDETGMPKERFSAKCLPNSPCSLEIQATKLEDSAVYFCASSQSRSLRGTEAFFGQGTRL
TVVEdlnkvfppevavfepseaeishtqkatlvclatgffpdhvelswwvngkevhsgvstdpqpl
keqpalndsryclssrlrvsatfwqnpmnhfrcqvqfyglsendewtqdrakpvtqivsaeawgra
dcgftsvsyqqgvlsatilyeillgkatlyavlvsalvlmamvkrkdf
MAGEC2 TCR 8-3 HM codon optimized sequence
Alpha chain:
TRAV24*01F/TRAJ32*02/codon-optimized mouse TRAC
Alpha chain DNA sequence
ATGGAGAAGAATCCTTTGGCAGCCCCACTTCTTATCCTCTGGTTTCATCTTGACTGCGTGAGCAGC
ATTCTGAACGTGGAACAAAGTCCTCAGTCACTGCATGTTCAGGAGGGAGACAGCACCAATTTCACC
TGCAGCTTCCCTTCCAGCAATTTTTATGCCCTTCACTGGTACAGATGGGAAACTGCAAAAAGCCCC
GAGGCCTTGTTTGTTATGACTCTTAATGGGGATGAAAAGAAGAAAGGACGCATTAGTGCCACTCTT
AATACCAAGGAGGGTTACAGCTATTTGTATATCAAAGGATCCCAGCCTGAGGACTCAGCCACATAC
CTCTGTGCCTCCGGAAGTGGTGGTGCTACAAACAAGCTCATCTTTGGAACTGGCACTCTGCTTGCT
GTCCAGCCAAacattcaaaacccagaacccgccgtctaccagctgaaagacccgaggtctcaagac
tctacgttgtgcttgttcaccgatttcgacagtcagataaatgtgcctaagaccatggagagtggc
actttcatcactgacaaatgtgtgttggacatgaaggctatggacagcaagtcaaacggcgcgatt
gcttggtccaaccaaacttctttcacgtgccaggacatcttcaaggagacaaacgccacctatcca
tcctctgatgttccgtgcgatgcgactcttaccgagaaaagcttcgagacggacatgaacttgaac
ttccaaaacctgcttgtgatggtactgcgaatacttcttcttaaggtggcgggcttcaatttgctc
atgacactcagactttggtctagc
Alpha chain protein sequence
MEKNPLAAPLLILWFHLDCVSSILNVEQSPQSLHVQEGDSTNFTCSFPSSNFYALHWYRWETAKSP
EALFVMTLNGDEKKKGRISATLNTKEGYSYLYIKGSQPEDSATYLCASGSGGATNKLIFGTGTLLA
VQPNiqnpepavyqlkdprsqdstlclftdfdsqinvpktmesgtfitdkcvldmkamdsksngai
awsnqtsftcqdifketnatypssdvpcdatlteksfetdmnlnfqnllvmvlrilllkvagfnll
mtlrlwss
Beta chain:
TRBV16*01/TRBJ1-1*01/codon-optimized mouse TRBC
Beta chain DNA sequence
ATGAGCCCAATTTTCACCTGCATCACAATCCTTTGTCTGCTGGCTGCAGGTTCTCCTGGTGAAGAA
GTCGCCCAGACTCCAAAACATCTTGTCAGAGGGGAAGGACAGAAAGCAAAACTTTATTGTGCCCCA
ATTAAAGGACACAGTTATGTTTTCTGGTACCAACAGGTCCTGAAAAACGAGTTCAAGTTCTTGATT
TCCTTCCAGAATGAAAATGTCTTTGATGAAACAGGTATGCCCAAGGAAAGATTTTCAGCTAAGTGC
CTCCCAAATTCACCCTGTAGCCTTGAGATCCAGGCTACTAAGCTTGAGGATTCAGCAGTGTATTTT
TGTGCCAGCAGCCAATCACGGAGCCTTAGGGGCACTGAAGCTTTCTTTGGACAAGGCACCAGACTC
ACAGTTGTTGaagatcttcgaaacgtaacccctccaaaagtgagtctctttgaaccgagtaaggct
gagatcgcgaacaaacaaaaggcgaccctcgtctgtcttgcgcgaggattttttcccgaccacgtg
gagttgtcttggtgggtaaacggtaaggaagtacacagcggtgtttgcaccgaccctcaagcctac
aaggaatctaactattcatactgcctttcatcccgacttagggtttctgctaccttttggcacaat
ccgaggaatcactttaggtgtcaagtacagttccacggattgtcagaggaggataaatggccggag
ggctccccgaagccggttacgcagaacattagtgcggaagcctggggacgagcagactgcggtatc
acgtctgccagctatcagcaaggcgttctgtcagcgacaattctgtacgaaatacttttgggtaag
gctacattgtatgcggtattggtgtctacgctggtagtcatggccatggtgaaacgaaaaaactca
Beta chain protein sequence
MSPIFTCITILCLLAAGSPGEEVAQTPKHLVRGEGQKAKLYCAPIKGHSYVFWYQQVLKNEFKFLI
SFQNENVFDETGMPKERFSAKCLPNSPCSLEIQATKLEDSAVYFCASSQSRSLRGTEAFFGQGTRL
TVVEdlrnvtppkvslfepskaeiankqkatlvclargffpdhvelswwvngkevhsgvctdpqay
kesnysyclssrlrvsatfwhnprnhfrcqvqfhglseedkwpegspkpvtqnisaeawgradcgi
tsasyqqgvlsatilyeillgkatlyavlvstlvvmamvkrkns
Complete Beta and Alpha ORF DNA Sequence (The underlined
italic region in the “Furin-P2A” site encodes a sequence
allowing for expression of two polypeptide chains in a
single cassette”)
ATGAGCCCAATTTTCACCTGCATCACAATCCTTTGTCTGCTGGCTGCAGGTTCTCCTGGTGAAGAA
GTCGCCCAGACTCCAAAACATCTTGTCAGAGGGGAAGGACAGAAAGCAAAACTTTATTGTGCCCCA
ATTAAAGGACACAGTTATGTTTTCTGGTACCAACAGGTCCTGAAAAACGAGTTCAAGTTCTTGATT
TCCTTCCAGAATGAAAATGTCTTTGATGAAACAGGTATGCCCAAGGAAAGATTTTCAGCTAAGTGC
CTCCCAAATTCACCCTGTAGCCTTGAGATCCAGGCTACTAAGCTTGAGGATTCAGCAGTGTATTTT
TGTGCCAGCAGCCAATCACGGAGCCTTAGGGGCACTGAAGCTTTCTTTGGACAAGGCACCAGACTC
ACAGTTGTTGaagatcttcgaaacgtaacccctccaaaagtgagtctctttgaaccgagtaaggct
gagatcgcgaacaaacaaaaggcgaccctcgtctgtcttgcgcgaggattttttcccgaccacgtg
gagttgtcttggtgggtaaacggtaaggaagtacacagcggtgtttgcaccgaccctcaagcctac
aaggaatctaactattcatactgcctttcatcccgacttagggtttctgctaccttttggcacaat
ccgaggaatcactttaggtgtcaagtacagttccacggattgtcagaggaggataaatggccggag
ggctccccgaagccggttacgcagaacattagtgcggaagcctggggacgagcagactgcggtatc
acgtctgccagctatcagcaaggcgttctgtcagcgacaattctgtacgaaatacttttgggtaag
gctacattgtatgcggtattggtgtctacgctggtagtcatggccatggtgaaacgaaaaaactca
agagccaaaagaagcgggagcggtgcgacaaactttagcctgttgaaacaagccggcgacgttgaa
gagaaccccggacctATGGAGAAGAATCCTTTGGCAGCCCCACTTCTTATCCTCTGGTTTCATCTT
GACTGCGTGAGCAGCATTCTGAACGTGGAACAAAGTCCTCAGTCACTGCATGTTCAGGAGGGAGAC
AGCACCAATTTCACCTGCAGCTTCCCTTCCAGCAATTTTTATGCCCTTCACTGGTACAGATGGGAA
ACTGCAAAAAGCCCCGAGGCCTTGTTTGTTATGACTCTTAATGGGGATGAAAAGAAGAAAGGACGC
ATTAGTGCCACTCTTAATACCAAGGAGGGTTACAGCTATTTGTATATCAAAGGATCCCAGCCTGAG
GACTCAGCCACATACCTCTGTGCCTCCGGAAGTGGTGGTGCTACAAACAAGCTCATCTTTGGAACT
GGCACTCTGCTTGCTGTCCAGCCAAacattcaaaacccagaacccgccgtctaccagctgaaagac
ccgaggtctcaagactctacgttgtgcttgttcaccgatttcgacagtcagataaatgtgcctaag
accatggagagtggcactttcatcactgacaaatgtgtgttggacatgaaggctatggacagcaag
tcaaacggcgcgattgcttggtccaaccaaacttctttcacgtgccaggacatcttcaaggagaca
aacgccacctatccatcctctgatgttccgtgcgatgcgactcttaccgagaaaagcttcgagacg
gacatgaacttgaacttocaaaacctgcttgtgatggtactgcgaatacttcttcttaaggtggcg
ggcttcaatttgctcatgacactcagactttggtctagc
Complete Beta and Alpha ORF Protein Sequence (The underlined
italic region in the “Furin-P2A” site allows expression of
two polypeptide chains in a single cassette”)”)
MSPIFTCITILCLLAAGSPGEEVAQTPKHLVRGEGQKAKLYCAPIKGHSYVFWYQQVLKNEFKFLI
SFQNENVFDETGMPKERFSAKCLPNSPCSLEIQATKLEDSAVYFCASSQSRSLRGTEAFFGQGTRL
TVVEdlmvtppkvslfepskaciankqkatlvclargffpdhvelswwvngkevhsgvctdpqayk
esnysyclssrlrvsatfwhnprnhfrcqvqfhglseedkwpegspkpvtqnisaeawgradcgit
sasyqqgvlsatilyeillgkatlyavlvstlvvmamvkrknsrakrsgsgatnfsllkgagdvee
npgpMEKNPLAAPLLILWFHLDCVSSILNVEQSPQSLHVQEGDSTNFTCSFPSSNFYALHWYRWET
AKSPEALFVMTLNGDEKKKGRISATLNTKEGYSYLYIKGSQPEDSATYLCASGSGGATNKLIFGTG
TLLAVQPNiqnpepavyqlkdprsqdstlclftdfdsqinvpktmesgtfitdkcvldmkamdsks
ngaiawsnqtsftcqdifketnatypssdvpcdatlteksfetdmnlnfqnllvmvlrillikvag
fnlimtlrlwss
MAGEC2 TCR 8-3 MGTM codon optimized sequence
Alpha chain:
TRAV24*01 F/TRAJ32*02/MGTM modified TRAC
Alpha chain DNA sequence
ATGGAGAAGAATCCTTTGGCAGCCCCACTTCTTATCCTCTGGTTTCATCTTGACTGCGTGAGCAGC
ATTCTGAACGTGGAACAAAGTCCTCAGTCACTGCATGTTCAGGAGGGAGACAGCACCAATTTCACC
TGCAGCTTCCCTTCCAGCAATTTTTATGCCCTTCACTGGTACAGATGGGAAACTGCAAAAAGCCCC
GAGGCCTTGTTTGTTATGACTCTTAATGGGGATGAAAAGAAGAAAGGACGCATTAGTGCCACTCTT
AATACCAAGGAGGGTTACAGCTATTTGTATATCAAAGGATCCCAGCCTGAGGACTCAGCCACATAC
CTCTGTGCCTCCGGAAGTGGTGGTGCTACAAACAAGCTCATCTTTGGAACTGGCACTCTGCTTGCT
GTCCAGCCAAacatccagaaccccgaccccgccgtgtaccagctgagggactccaagtccagcgac
aagagcgtgtgtctgtttacggacttcgacagccagaccaacgtgagtcaaagcaaggacagcgac
gtctacataacggataagaccgtgctggacatgcggagcatggacttcaagagcaacagcgccgtg
gcctggtccaacaagagcgacttcgcctgcgccaacgccttcaacaacagcatcatccccgaggac
accttettccccagcagegacgtgccctgcgacgtgaaactggtggagaagtccttegagacagac
accaatctgaactttcagaacctgctggtgategtgctgeggattctgctgctgaaagtggccggc
ttcaatctgctgatgaccctgcggctgtggagcagc
Alpha chain protein sequence
MEKNPLAAPLLILWFHLDCVSSILNVEQSPQSLHVQEGDSTNFTCSFPSSNFYALHWYRWETAKSP
EALFVMTLNGDEKKKGRISATLNTKEGYSYLYIKGSQPEDSATYLCASGSGGATNKLIFGTGTLLA
VQPNiqnpdpavyqlrdskssdksvclftdfdsqtnvsqskdsdvyitdktvldmrsmdfksnsav
awsnksdfacanafnnsiipedtffpssdvpcdvklveksfetdtnlnfqnllvivlrilllkvag
fnllmtlrlwss
Beta chain:
TRBV16*01/TRBJ1-1*01/MGTM modified TRBC
Beta chain DNA sequence
ATGAGCCCAATTTTCACCTGCATCACAATCCTTTGTCTGCTGGCTGCAGGTTCTCCTGGTGAAGAA
GTCGCCCAGACTCCAAAACATCTTGTCAGAGGGGAAGGACAGAAAGCAAAACTTTATTGTGCCCCA
ATTAAAGGACACAGTTATGTTTTCTGGTACCAACAGGTCCTGAAAAACGAGTTCAAGTTCTTGATT
TCCTTCCAGAATGAAAATGTCTTTGATGAAACAGGTATGCCCAAGGAAAGATTTTCAGCTAAGTGC
CTCCCAAATTCACCCTGTAGCCTTGAGATCCAGGCTACTAAGCTTGAGGATTCAGCAGTGTATTTT
TGTGCCAGCAGCCAATCACGGAGCCTTAGGGGCACTGAAGCTTTCTTTGGACAAGGCACCAGACTC
ACAGTTGTTGaagatctgaacaaggtgttccctccagaggtggccgtgttcgagccttctaaggcc
gagatcgcccacacacaaaaagccaccctcgtgtgcctggccaccggctttttccccgaccacgtg
gaactgtcttggtgggtcaacggcaaagaggtgcactccggcgtgtcaacggatccccagcctctg
aaagaacagcctgccctgaacgacagccggtactgcctgagctccagactgagagtgtccgccacc
ttctggcagaacccccggaaccacttcagatgccaggtgcagttttacggcctgagcgagaacgac
gagtggacccaggacagagccaagcccgtgacacaaatcgtgtctgccgaagcctggggaagagcc
gattgcggcatcaccagcgcctcctatcaccagggcgtgctgagcgccacaatcctgtacgaaatc
ctgctgggcaaggccaccctgtacgccgtgctggtgtctgctctggtgctgatggccatggtcaag
cggaaggacttt
Beta chain protein sequence
MSPIFTCITILCLLAAGSPGEEVAQTPKHLVRGEGQKAKLYCAPIKGHSYVFWYQQVLKNEFKFLI
SFQNENVFDETGMPKERFSAKCLPNSPCSLEIQATKLEDSAVYFCASSQSRSLRGTEAFFGQGTRL
TVVEdlnkvfppevavfepskaeiahtqkatlvclatgffpdhvelswwvngkevhsgvstdpqpl
keqpalndsryclssrlrvsatfwqnpmhfreqvqfyglsendewtqdrakpvtqivsaeawgrad
cgitsasyhqgvlsatilyeillgkatlyavlvsalvlmamvkrkdf
Complete Beta and Alpha ORF DNA Sequence
ATGAGCCCAATTTTCACCTGCATCACAATCCTTTGTCTGCTGGCTGCAGGTTCTCCTGGTGAAGAA
GTCGCCCAGACTCCAAAACATCTTGTCAGAGGGGAAGGACAGAAAGCAAAACTTTATTGTGCCCCA
ATTAAAGGACACAGTTATGTTTTCTGGTACCAACAGGTCCTGAAAAACGAGTTCAAGTTCTTGATT
TCCTTCCAGAATGAAAATGTCTTTGATGAAACAGGTATGCCCAAGGAAAGATTTTCAGCTAAGTGC
CTCCCAAATTCACCCTGTAGCCTTGAGATCCAGGCTACTAAGCTTGAGGATTCAGCAGTGTATTTT
TGTGCCAGCAGCCAATCACGGAGCCTTAGGGGCACTGAAGCTTTCTTTGGACAAGGCACCAGACTC
ACAGTTGTTGaagatctgaacaaggtgttccctccagaggtggccgtgttcgagccttctaaggcc
gagatcgcccacacacaaaaagccaccctcgtgtgcctggccaccggctttttccccgaccacgtg
gaactgtcttggtgggtcaacggcaaagaggtgcactccggcgtgtcaacggatccccagcctctg
aaagaacagcctgccctgaacgacagccggtactgcctgagctccagactgagagtgtccgccacc
ttctggcagaacccccggaaccacttcagatgccaggtgcagttttacggcctgagcgagaacgac
gagtggacccaggacagagccaagcccgtgacacaaatcgtgtctgccgaagcctggggaagagcc
gattgcggcatcaccagcgcctcctatcaccagggcgtgctgagcgccacaatcctgtacgaaatc
ctgctgggcaaggccaccctgtacgccgtgctggtgtctgctctggtgctgatggccatggtcaag
cggaaggactttggcagcggcagagccaaaagaagcgggagcggtgcgacaaactttagcctgttg
aaacaagccggcgaccttgaagagaaccccggacctATGGAGAAGAATCCTTTGGCAGCCCCACTT
CTTATCCTCTGGTTTCATCTTGACTGCGTGAGCAGCATTCTGAACGTGGAACAAAGTCCTCAGTCA
CTGCATGTTCAGGAGGGAGACAGCACCAATTTCACCTGCAGCTTCCCTTCCAGCAATTTTTATGCC
CTTCACTGGTACAGATGGGAAACTGCAAAAAGCCCCGAGGCCTTGTTTGTTATGACTCTTAATGGG
GATGAAAAGAAGAAAGGACGCATTAGTGCCACTCTTAATACCAAGGAGGGTTACAGCTATTTGTAT
ATCAAAGGATCCCAGCCTGAGGACTCAGCCACATACCTCTGTGCCTCCGGAAGTGGTGGTGCTACA
AACAAGCTCATCTTTGGAACTGGCACTCTGCTTGCTGTCCAGCCAAacatccagaaccccgacccc
gccgtgtaccagctgagggactccaagtccagcgacaagagcgtgtgtctgtttacggacttcgac
agccagaccaacgtgagtcaaagcaaggacagcgacgtctacataacggataagaccgtgctggac
atgcggagcatggacttcaagagcaacagcgccgtggcctggtccaacaagagcgacttcgcctgc
gccaacgccttcaacaacagcatcatccccgaggacaccttcttccccagcagcgacgtgccctgc
gacgtgaaactggtggagaagtccttcgagacagacaccaatctgaactttcagaacctgctggtg
atcgtgctgcggattctgctgctgaaagtggccggcttcaatctgctgatgaccctgcggctgtgg
agcagc
Complete Beta and Alpha ORF Protein Sequence
MSPIFTCITILCLLAAGSPGEEVAQTPKHLVRGEGQKAKLYCAPIKGHSYVFWYQQVLKNEFKFLI
SFQNENVFDETGMPKERFSAKCLPNSPCSLEIQATKLEDSAVYFCASSQSRSLRGTEAFFGQGTRL
TVVEdlnkvfppevavfepskaeiahtqkatlvclatgffpdhvelswwvngkevhsgvstdpqpl
keqpalndsryclssrlrvsatfwqnprnhfreqvqfyglsendewtqdrakpvtqivsaeawgra
dcgitsasyhqgvlsatilyeillgkatlyavlvsalvlmamvkrkdfgsgrakrsgsgatnfsll
kgagdveenpgpMEKNPLAAPLLILWFHLDCVSSILNVEQSPQSLHVQEGDSTNFTCSFPSSNFYA
LHWYRWETAKSPEALFVMTLNGDEKKKGRISATLNTKEGYSYLYIKGSQPEDSATYLCASGSGGAT
NKLIFGTGTLLAVQPNiqnpdpavyqlrdskssdksvclftdfdsqtnvsqskdsdvyitdktvld
mrsmdfksnsavawsnksdfacanafnnsiipedtffpssdvpcdvklveksfetdtnlnfqnllv
ivlrilllkvagfnllmtlrlwss

TABLE 2B
TCR sequences recognizing a MAGEC2 antigen presented by HLA serotype HLA-
A*24

MAGEC2 TCR 4-58 wild type sequence
Alpha chain:
TRAV10/TRAJ39/TRAC
Alpha chain DNA sequence
ATGAAAAAGCATCTGACGACCTTCTTGGTGATTTTGTGGCTTTATTTTTATAGGG
GGAATGGCAAAAACCAAGTGGAGCAGAGTCCTCAGTCCCTGATCATCCTGGAG
GGAAAGAACTGCACTCTTCAATGCAATTATACAGTGAGCCCCTTCAGCAACTT
AAGGTGGTATAAGCAAGATACGGGGAGAGGTCCTGTTTCCCTGACAATCATGA
CTTTCAGTGAGAACACAAAGTCGAACGGAAGATATACAGCAACTCTGGATGC
AGACACAAAGCAAAGCTCTCTGCACATCACAGCCTCCCAGCTCAGCGATTCAG
CCTCCTACATCtgcGTCGTGTCTGCCCGCAACGCAGGTAACATGCTTACATTC
GGAGGGGGAACAAGGTTAATGGTCAAACCCCatatccagaaccctgaccctgccgtgtaccagctga
gagactctaaatccagtgacaagtctgtctgcctattcaccgattttgattctcaaacaaatgtgtcacaaagtaaggattctgatgtgta
tatcacagacaaaactgtgctagacatgaggtctatggacttcaagagcaacagtgctgtggcctggagcaacaaatctgactttgc
atgtgcaaacgccttcaacaacagcattattccagaagacaccttcttccccagcccagaaagttcctgtgatgtcaagctggtcgag
aaaagctttgaaacagatacgaacctaaactttcaaaacctgtcagtgattgggttccgaatcctcctcctgaaagtggccgggtttaa
tctgctcatgacgctgcggctgtggtccagc
alpha chain protein sequence
MKKHLTTFLVILWLYFYRGNGKNQVEQSPQSLIILEGKNCTLQCNYTVSPFSNLRW
YKQDTGRGPVSLTIMTFSENTKSNGRYTATLDADTKQSSLHITASQLSDSASYICV
VSARNAGNMLTFGGGTRLMVKPHiqnpdpavyqlrdskssdksvclftdfdsqtnvsqskdsdvyitdkt
vldmrsmdfksnsavawsnksdfacanafnnsiipedtffpspesscdvklveksfetdtnlnfqnlsvigfrilllkvagfnllm
tlrlwss
Beta chain:
TRBV27/TRAB2-1/TRBC1
Beta chain DNA sequence
ATGGGCCCCCAGCTCCTTGGCTATGTGGTCCTTTGCCTTCTAGGAGCAGGCCCC
CTGGAAGCCCAAGTGACCCAGAACCCAAGATACCTCATCACAGTGACTGGAAA
GAAGTTAACAGTGACTTGTTCTCAGAATATGAACCATGAGTATATGTCCTGGT
ATCGACAAGACCCAGGGCTGGGCTTAAGGCAGATCTACTATTCAATGAATGTT
GAGGTGACTGATAAGGGAGATGTTCCTGAAGGGTACAAAGTCTCTCGAAAAG
AGAAGAGGAATTTCCCCCTGATCCTGGAGTCGCCCAGCCCCAACCAGACCTCTC
TGTACTTCTGTGCCtgCGCTAGTAGCTTCGGCACCAGCGGTCGCGGTGAACA
GTTTTTCGGGCCAGGGACACGGCTCACCGTGCTAGaggacctgaacaaggtgttcccacccgag
gtcgctgtgtttgagccatcagaagcagagatctcccacacccaaaaggccacactggtgtgcctggccacaggcttcttccctgac
cacgtggagctgagctggtgggtgaatgggaaggaggtgcacagtggggtcagcacggacccgcagcccctcaaggagcagc
ccgccctcaatgactccagatactgcctgagcagccgcctgagggtctcggccaccttctggcagaacccccgcaaccacttccg
ctgtcaagtccagttctacgggctctcggagaatgacgagtggacccaggatagggccaaacccgtcacccagatcgtcagcgcc
gaggcctggggtagagcagactgtggctttacctcggtgtcctaccagcaaggggtcctgtctgccaccatcctctatgagatcctg
ctagggaaggccaccctgtatgctgtgctggtcagcgcccttgtgttgatggccatggtcaagagaaaggatttc
Beta chain protein sequence
MGPQLLGYVVLCLLGAGPLEAQVTQNPRYLITVTGKKLTVTCSQNMNHEYMSWY
RQDPGLGLRQIYYSMNVEVTDKGDVPEGYKVSRKEKRNFPLILESPSPNQTSLYFC
ACASSFGTSGRGEqFFGPGTRLTVLEdlnkvfppevavfepseaeishtqkatlvclatgffpdhvelsww
vngkevhsgvstdpqplkeqpalndsryclssrlrvsatfwqnprnhfrcqvqfyglsendewtqdrakpvtqivsacawgra
degftsvsyqqgvlsatilyeillgkatlyavlvsalvlmamvkrkdf
MAGEC2 TCR 4-58 HM codon optimized sequence
Alpha chain:
TRAV10/TRAJ39/codon-optimized mouse TRAC
Alpha chain DNA sequence
ATGAAAAAGCATCTGACGACCTTCTTGGTGATTTTGTGGCTTTATTTTTATAGGG
GGAATGGCAAAAACCAAGTGGAGCAGAGTCCTCAGTCCCTGATCATCCTGGAG
GGAAAGAACTGCACTCTTCAATGCAATTATACAGTGAGCCCCTTCAGCAACTT
AAGGTGGTATAAGCAAGATACGGGGAGAGGTCCTGTTTCCCTGACAATCATGA
CTTTCAGTGAGAACACAAAGTCGAACGGAAGATATACAGCAACTCTGGATGC
AGACACAAAGCAAAGCTCTCTGCACATCACAGCCTCCCAGCTCAGCGATTCAG
CCTCCTACATCtgcGTCGTGTCTGCCCGCAACGCAGGTAACATGCTTACATTC
GGAGGGGGAACAAGGTTAATGGTCAAACCCCatattcaaaacccagaacccgccgtctaccagctga
aagacccgaggtctcaagactctacgttgtgcttgttcaccgatttcgacagtcagataaatgtgcctaagaccatggagagtggcac
tttcatcactgacaaatgtgtgttggacatgaaggctatggacagcaagtcaaacggcgcgattgcttggtccaaccaaacttctttca
cgtgccaggacatcttcaaggagacaaacgccacctatccatcctctgatgttccgtgcgatgcgactcttaccgagaaaagcttcg
agacggacatgaacttgaacttccaaaacctgcttgtgatggtactgcgaatacttcttcttaaggtggcgggcttcaatttgctcatga
cactcagactttggtctagc
Alpha chain protein sequence
MKKHLTTFLVILWLYFYRGNGKNQVEQSPQSLIILEGKNCTLQCNYTVSPFSNLRW
YKQDTGRGPVSLTIMTFSENTKSNGRYTATLDADTKQSSLHITASQLSDSASYICV
VSARNAGNMLTFGGGTRLMVKPHiqnpepavyqlkdprsqdstlclftdfdsqinvpktmesgtfitdkcv
ldmkamdsksngaiawsnqtsftcqdifketnatypssdvpcdatlteksfetdmnlnfqnllvmvlrilllkvagfnllmtlrl
WSS
Beta chain:
TRBV27/TRAB2-1/codon-optimized mouse TRBC
Beta chain DNA sequence
ATGGGCCCCCAGCTCCTTGGCTATGTGGTCCTTTGCCTTCTAGGAGCAGGCCCC
CTGGAAGCCCAAGTGACCCAGAACCCAAGATACCTCATCACAGTGACTGGAAA
GAAGTTAACAGTGACTTGTTCTCAGAATATGAACCATGAGTATATGTCCTGGT
ATCGACAAGACCCAGGGCTGGGCTTAAGGCAGATCTACTATTCAATGAATGTT
GAGGTGACTGATAAGGGAGATGTTCCTGAAGGGTACAAAGTCTCTCGAAAAG
AGAAGAGGAATTTCCCCCTGATCCTGGAGTCGCCCAGCCCCAACCAGACCTCTC
TGTACTTCTGTGCCtgCGCTAGTAGCTTCGGCACCAGCGGTCGCGGTGAACA
GTTTTTCGGGCCAGGGACACGGCTCACCGTGCTAGaagatcttcgaaacgtaacccctccaaaa
gtgagtctctttgaaccgagtaaggctgagatcgcgaacaaacaaaaggcgaccctcgtctgtcttgcgcgaggattttttcccgacc
acgtggagttgtcttggtgggtaaacggtaaggaagtacacagcggtgtttgcaccgaccctcaagcctacaaggaatctaactatt
catactgcctttcatcccgacttagggtttctgctaccttttggcacaatccgaggaatcactttaggtgtcaagtacagttccacggatt
gtcagaggaggataaatggccggagggctccccgaagccggttacgcagaacattagtgcggaagcctggggacgagcagact
gcggtatcacgtctgccagctatcagcaaggcgttctgtcagcgacaattctgtacgaaatacttttgggtaaggctacattgtatgcg
gtattggtgtctacgctggtagtcatggccatggtgaaacgaaaaaactca
Beta chain protein sequence
MGPQLLGYVVLCLLGAGPLEAQVTQNPRYLITVTGKKLTVTCSQNMNHEYMSWY
RQDPGLGLRQIYYSMNVEVTDKGDVPEGYKVSRKEKRNFPLILESPSPNQTSLYFC
ACASSFGTSGRGEOFFGPGTRLTVLEdlrnvtppkvslfepskaeiankqkatlvclargffpdhvelsww
vngkevhsgvctdpqaykesnysyclssrlrvsatfwhnprnhfrcqvqfhglseedkwpegspkpvtqnisaeawgradc
gitsasyqqgvlsatilyeillgkatlyavlvstlvvmamvkrkns
Complete Beta and Alpha ORF DNA Sequence (The underlined italic region in the “Furin-
P2A” site encodes a sequence allowing for expression of two polypeptide chains in a single
cassette”)
ATGGGCCCCCAGCTCCTTGGCTATGTGGTCCTTTGCCTTCTAGGAGCAGGCCCC
CTGGAAGCCCAAGTGACCCAGAACCCAAGATACCTCATCACAGTGACTGGAAA
GAAGTTAACAGTGACTTGTTCTCAGAATATGAACCATGAGTATATGTCCTGGT
ATCGACAAGACCCAGGGCTGGGCTTAAGGCAGATCTACTATTCAATGAATGTT
GAGGTGACTGATAAGGGAGATGTTCCTGAAGGGTACAAAGTCTCTCGAAAAG
AGAAGAGGAATTTCCCCCTGATCCTGGAGTCGCCCAGCCCCAACCAGACCTCTC
TGTACTTCTGTGCCtgCGCTAGTAGCTTCGGCACCAGCGGTCGCGGTGAACA
GTTTTTCGGGCCAGGGACACGGCTCACCGTGCTAGaagatcttcgaaacgtaacccctccaaaa
gtgagtctctttgaaccgagtaaggctgagatcgcgaacaaacaaaaggcgaccctcgtctgtcttgcgcgaggattttttcccgacc
acgtggagttgtcttggtgggtaaacggtaaggaagtacacagcggtgtttgcaccgaccctcaagcctacaaggaatctaactatt
catactgcctttcatcccgacttagggtttctgctaccttttggcacaatccgaggaatcactttaggtgtcaagtacagttccacggatt
gtcagaggaggataaatggccggagggctccccgaagccggttacgcagaacattagtgcggaagcctggggacgagcagact
gcggtatcacgtctgccagctatcagcaaggcgttctgtcagcgacaattctgtacgaaatacttttgggtaaggctacattgtatgcg
gtattggtgtctacgctggtagtcatggccatggtgaaacgaaaaaactcaagagccaaaagaagcgggagcggtgcgacaaa
ctttagcctgttgaaacaagccggcgacgttgaagagaaccccggacctATGAAAAAGCATCTGACGACCT
TCTTGGTGATTTTGTGGCTTTATTTTTATAGGGGGAATGGCAAAAACCAAGTGG
AGCAGAGTCCTCAGTCCCTGATCATCCTGGAGGGAAAGAACTGCACTCTTCAAT
GCAATTATACAGTGAGCCCCTTCAGCAACTTAAGGTGGTATAAGCAAGATAC
GGGGAGAGGTCCTGTTTCCCTGACAATCATGACTTTCAGTGAGAACACAAAG
TCGAACGGAAGATATACAGCAACTCTGGATGCAGACACAAAGCAAAGCTCTCT
GCACATCACAGCCTCCCAGCTCAGCGATTCAGCCTCCTACATCtgcGTCGTGTCT
GCCCGCAACGCAGGTAACATGCTTACATTCGGAGGGGGAACAAGGTTAATG
GTCAAACCCCatattcaaaacccagaacccgccgtctaccagctgaaagacccgaggtctcaagactctacgttgtgcttg
ttcaccgatttcgacagtcagataaatgtgcctaagaccatggagagtggcactttcatcactgacaaatgtgtgttggacatgaagg
ctatggacagcaagtcaaacggcgcgattgcttggtccaaccaaacttctttcacgtgccaggacatcttcaaggagacaaacgcca
cctatccatcctctgatgttccgtgcgatgcgactcttaccgagaaaagcttcgagacggacatgaacttgaacttccaaaacctgctt
gtgatggtactgcgaatacttcttcttaaggtgggggcttcaatttgctcatgacactcagactttggtctagc
Complete Beta and Alpha ORF Protein Sequence (The underlined italic region in the
“Furin-P2A” site allows expression of two polypeptide chains in a single cassette”)”)
MGPQLLGYVVLCLLGAGPLEAQVTQNPRYLITVTGKKLTVTCSQNMNHEYMSWY
RQDPGLGLRQIYYSMNVEVTDKGDVPEGYKVSRKEKRNFPLILESPSPNQTSLYFC
ACASSFGTSGRGEQFFGPGTRLTVLEdlrnvtppkvslfepskaciankqkatlvclargffpdhvelsww
vngkevhsgvctdpqaykesnysyclssrlrvsatfwhnprnhfrcqvqfhglseedkwpegspkpvtqnisaeawgradc
gitsasyqqgvlsatilyeillgkatlyavlvstlvvmamvkrknsrakrsgsgatnfsllkqagdveenpgpMKKHLTTF
LVILWLYFYRGNGKNQVEQSPQSLIILEGKNCTLQCNYTVSPFSNLRWYKQDTGRG
PVSLTIMTFSENTKSNGRYTATLDADTKQSSLHITASQLSDSASYICVVSARNAGN
MLTFGGGTRLMVKPHiqnpepavyqlkdprsqdstlclftdfdsqinvpktmesgtfitdkcvldmkamdsksn
gaiawsnqtsftcqdifketnatypssdvpcdatlteksfetdmnlnfqnllvmvlrilllkvagfnllmtlrlwss
MAGEC2 TCR 4-58 MGTM codon optimized sequence
Alpha chain:
TRAV10/TRAJ39/MGTM modified TRAC
Alpha chain DNA sequence
ATGAAAAAGCATCTGACGACCTTCTTGGTGATTTTGTGGCTTTATTTTTATAGGG
GGAATGGCAAAAACCAAGTGGAGCAGAGTCCTCAGTCCCTGATCATCCTGGAG
GGAAAGAACTGCACTCTTCAATGCAATTATACAGTGAGCCCCTTCAGCAACTT
AAGGTGGTATAAGCAAGATACGGGGAGAGGTCCTGTTTCCCTGACAATCATGA
CTTTCAGTGAGAACACAAAGTCGAACGGAAGATATACAGCAACTCTGGATGC
AGACACAAAGCAAAGCTCTCTGCACATCACAGCCTCCCAGCTCAGCGATTCAG
CCTCCTACATCtgcGTCGTGTCTGCCCGCAACGCAGGTAACATGCTTACATTC
GGAGGGGGAACAAGGTTAATGGTCAAACCCCatatccagaaccccgaccccgccgtgtaccagctg
agggactccaagtccagcgacaagagcgtgtgtctgtttacggacttcgacagccagaccaacgtgagtcaaagcaaggacagc
gacgtctacataacggataagaccgtgctggacatgcggagcatggacttcaagagcaacagcgccgtggcctggtccaacaag
agcgacttcgcctgcgccaacgccttcaacaacagcatcatccccgaggacaccttcttccccagcagcgacgtgccctgcgacgt
gaaactggtggagaagtccttcgagacagacaccaatctgaactttcagaacctgctggtgatcgtgctgcggattctgctgctgaa
agtggccggcttcaatctgctgatgaccctgcggctgtggagcagc
Alpha chain protein sequence
MKKHLTTFLVILWLYFYRGNGKNQVEQSPQSLIILEGKNCTLQCNYTVSPFSNLRW
YKQDTGRGPVSLTIMTFSENTKSNGRYTATLDADTKQSSLHITASQLSDSASYICV
VSARNAGNMLTFGGGTRLMVKPHiqnpdpavyqlrdskssdksvclftdfdsqtnvsqskdsdvyitdkt
vldmrsmdfksnsavawsnksdfacanafnnsiipedtffpssdvpcdvklveksfetdtnlnfqnllvivlrilllkvagfnllm
tlrlwss
Beta chain:
TRBV27/TRAB2-1/MGTM modified TRBC
Beta chain DNA sequence
ATGGGCCCCCAGCTCCTTGGCTATGTGGTCCTTTGCCTTCTAGGAGCAGGCCCC
CTGGAAGCCCAAGTGACCCAGAACCCAAGATACCTCATCACAGTGACTGGAAA
GAAGTTAACAGTGACTTGTTCTCAGAATATGAACCATGAGTATATGTCCTGGT
ATCGACAAGACCCAGGGCTGGGCTTAAGGCAGATCTACTATTCAATGAATGTT
GAGGTGACTGATAAGGGAGATGTTCCTGAAGGGTACAAAGTCTCTCGAAAAG
AGAAGAGGAATTTCCCCCTGATCCTGGAGTCGCCCAGCCCCAACCAGACCTCTC
TGTACTTCTGTGCCtgCGCTAGTAGCTTCGGCACCAGCGGTCGCGGTGAACA
GTTTTTCGGGCCAGGGACACGGCTCACCGTGCTAGaagatctgaacaaggtgttccctccagag
gtggccgtgttcgagccttctaaggccgagatcgcccacacacaaaaagccaccctcgtgtgcctggccaccggctttttccccga
ccacgtggaactgtcttggtgggtcaacggcaaagaggtgcactccggcgtgtcaacggatccccagcctctgaaagaacagcct
gccctgaacgacagccggtactgcctgagctccagactgagagtgtccgccaccttctggcagaacccccggaaccacttcagat
gccaggtgcagttttacggcctgagcgagaacgacgagtggacccaggacagagccaagcccgtgacacaaatcgtgtctgccg
aagcctggggaagagccgattgcggcatcaccagcgcctcctatcaccagggcgtgctgagcgccacaatcctgtacgaaatcct
gctgggcaaggccaccctgtacgccgtgctggtgtctgctctggtgctgatggccatggtcaagcggaaggacttt
Beta chain protein sequence
MGPQLLGYVVLCLLGAGPLEAQVTQNPRYLITVTGKKLTVTCSQNMNHEYMSWY
RQDPGLGLRQIYYSMNVEVTDKGDVPEGYKVSRKEKRNFPLILESPSPNQTSLYFC
ACASSFGTSGRGEQFFGPGTRLTVLEdlnkvfppevavfepskaeiahtqkatlvclatgffpdhvelsw
wvngkevhsgvstdpqplkeqpalndsryclssrlrvsatfwqnprnhfrcqvqfyglsendewtqdrakpvtqivsaeawg
radcgitsasyhqgvlsatilyeillgkatlyavlvsalvlmamvkrkdf
Complete Beta and Alpha ORF DNA Sequence
ATGGGCCCCCAGCTCCTTGGCTATGTGGTCCTTTGCCTTCTAGGAGCAGGCCCC
CTGGAAGCCCAAGTGACCCAGAACCCAAGATACCTCATCACAGTGACTGGAAA
GAAGTTAACAGTGACTTGTTCTCAGAATATGAACCATGAGTATATGTCCTGGT
ATCGACAAGACCCAGGGCTGGGCTTAAGGCAGATCTACTATTCAATGAATGTT
GAGGTGACTGATAAGGGAGATGTTCCTGAAGGGTACAAAGTCTCTCGAAAAG
AGAAGAGGAATTTCCCCCTGATCCTGGAGTCGCCCAGCCCCAACCAGACCTCTC
TGTACTTCTGTGCCtgCGCTAGTAGCTTCGGCACCAGCGGTCGCGGTGAACA
GTTTTTCGGGCCAGGGACACGGCTCACCGTGCTAGaagatctgaacaaggtgttccctccagag
gtggccgtgttcgagccttctaaggccgagatcgcccacacacaaaaagccaccctcgtgtgcctggccaccggctttttccccga
ccacgtggaactgtcttggtgggtcaacggcaaagaggtgcactccggcgtgtcaacggatccccagcctctgaaagaacagcct
gccctgaacgacagccggtactgcctgagctccagactgagagtgtccgccaccttctggcagaacccccggaaccacttcagat
gccaggtgcagttttacggcctgagcgagaacgacgagtggacccaggacagagccaagcccgtgacacaaatcgtgtctgccg
aagcctggggaagagccgattgggcatcaccagcgcctcctatcaccagggcgtgctgagcgccacaatcctgtacgaaatcct
gctgggcaaggccaccctgtacgccgtgctggtgtctgctctggtgctgatggccatggtcaagcggaaggactttggcagcggc
agagccaaaagaagcgggagcggtgcgacaaactttagcctgttgaaacaagccggcgacgttgaagagaaccccggacc
tATGAAAAAGCATCTGACGACCTTCTTGGTGATTTTGTGGCTTTATTTTTATAGG
GGGAATGGCAAAAACCAAGTGGAGCAGAGTCCTCAGTCCCTGATCATCCTGGA
GGGAAAGAACTGCACTCTTCAATGCAATTATACAGTGAGCCCCTTCAGCAAC
TTAAGGTGGTATAAGCAAGATACGGGGAGAGGTCCTGTTTCCCTGACAATCAT
GACTTTCAGTGAGAACACAAAGTCGAACGGAAGATATACAGCAACTCTGGAT
GCAGACACAAAGCAAAGCTCTCTGCACATCACAGCCTCCCAGCTCAGCGATTC
AGCCTCCTACATCtgcGTCGTGTCTGCCCGCAACGCAGGTAACATGCTTACAT
TCGGAGGGGGAACAAGGTTAATGGTCAAACCCCatatccagaaccccgaccccgccgtgtaccag
ctgagggactccaagtccagcgacaagagcgtgtgtctgtttacggacttcgacagccagaccaacgtgagtcaaagcaaggaca
gcgacgtctacataacggataagaccgtgctggacatgcggagcatggacttcaagagcaacagcgccgtggcctggtccaaca
agagcgacttcgcctgcgccaacgccttcaacaacagcatcatccccgaggacaccttcttccccagcagcgacgtgccctgcga
cgtgaaactggtggagaagtccttcgagacagacaccaatctgaactttcagaacctgctggtgatcgtgctgcggattctgctgctg
aaagtggccggcttcaatctgctgatgaccctgcggctgtggagcagc
Complete Beta and Alpha ORF Protein Sequence
MGPQLLGYVVLCLLGAGPLEAQVTQNPRYLITVTGKKLTVTCSQNMNHEYMSWY
RQDPGLGLRQIYYSMNVEVTDKGDVPEGYKVSRKEKRNFPLILESPSPNQTSLYFC
ACASSFGTSGRGEQFFGPGTRLTVLEdlnkvfppevavfepskaeiahtqkatlvclatgffpdhvelsw
wvngkevhsgvstdpqplkeqpalndsryclssrlrvsatfwqnprnhfrcqvqfyglsendewtqdrakpvtqivsaeawg
radcgitsasyhqgvlsatilyeillgkatlyavlvsalvlmamvkrkdfgsgrakrsgsgatnfsllkqagdveenpgpMKK
HLTTFLVILWLYFYRGNGKNQVEQSPQSLIILEGKNCTLQCNYTVSPFSNLRWYKQ
DTGRGPVSLTIMTFSENTKSNGRYTATLDADTKQSSLHITASQLSDSASYICVVSAR
NAGNMLTFGGGTRLMVKPHiqnpdpavyqlrdskssdksvclftdfdsqtnvsqskdsdvyitdktvldmrs
mdfksnsavawsnksdfacanafnnsiipedtffpssdvpcdvklveksfetdtnInfqnllvivlrilllkvagfnllmtlrlwss

Table 2, such as Table 2A and Table 2B, provides, in part, representative TCR sequences grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are shown in standard order of appearance from left (N-terminus) to right (C-terminus). TRAV, TRAJ, and TRAC genes for each TCR alpha chain described herein, and TRBV, TRBJ, and TRBC genes for each TCR beta chain described herein, are annotated according to well-known IMGT nomenclature described herein.

TABLE 3
Representative Human MAGEC2 cDNA sequence
atgcctcccgttocaggcgttccattccgcaacgttgacaacgactccccgacctcagttgagttagaagactgggtagatgcacag
catcccacagatgaggaagaggaggaagcctcctccgcctcttccactttgtacttagtattttccccctcttctttctccacatcc
tcttctctgattcttggtggtcctgaggaggaggaggtgccctctggtgtgataccaaatcttaccgagagcattoccagtagtcct
ccacagggtcctccacagggtccttcccagagtcctctgagctcctgctgctcctctttttcatggagctcattcagtgaggagtcc
agcagccagaaaggggaggatacaggcacctgtcagggcctgccagacagtgagtcctctttcacatatacactagatgaaaaggtg
gccgagttagtggagttcctgctcctcaaatacgaagcagaggagcctgtaacagaggcagagatgctgatgattgtcatcaagtac
aaagattactttcctgtgatactcaagagagcccgtgagttcatggagcttctttttggccttgccctgatagaagtgggccctgac
cacttctgtgtgtttgcaaacacagtaggcctcaccgatgagggtagtgatgatgagggcatgcccgagaacagcctcctgattatt
attctgagtgtgatcttcataaagggcaactgtgcctctgaggaggtcatctgggaagtgctgaatgcagtaggggtatatgctggg
agggagcacttcgtctatggggagcctagggagctcctcactaaagtttgggtgcagggacattacctggagtatcgggaggtgccc
cacagttctcctccatattatgaattcctgtggggtccgagagcccattcagaaagcatcaagaagaaagtactagagtttttagcc
aagctgaacaacactgttcctagttcctttccatcctggtacaaggatgctttgaaagatgtggaagagagagtccaggccacaatt
gataccgcagatgatgccactgtcatggccagtgaaagcctcagtgtcatgtccagcaacgtctccttttctgagtga
Representative Human MAGEC2 protein sequence
(Representative, non-limiting epitopes underlined)
MPPVPGVPFRNVDNDSPTSVELEDWVDAQHPTDEEEEEASSASSTLYLVFSPSSFSTSSSLILGGPEEEEVPSGVIPNLTESIPSSP
PQGPPQGPSQSPLSSCCSSFSWSSFSEESSSQKGEDTGTCQGLPDSESSFTYTLDEKVAELVEFLLLKYEAEEPVTEAEMLMIVIKY
KDYFPVILKRAREFMELLFGLALIEVGPDHFCVFANTVGLTDEGSDDEGMPENSLLIIILSVIFIKGNCASEEVIWEVLNAVGVYAG
REHFVYGEPRELLTKVWVQGHYLEYREVPHSSPPYYEFLWGPRAHSESIKKKVLEFLAKLNNTVPSSFPSWYKDALKDVEERVQATI
DTADDATVMASESLSVMSSNVSFSE
Representative Human HLA-B*07:02 DNA sequence
atgctggtcatggcgccccgaaccgtcctcctgctgctctcggcggccctggccctgaccgagacctgggccggctcccactccatg
aggtatttctacacctccgtgtcccggcccggccgcggggagccccgcttcatctcagtgggctacgtggacgacacccagttcgtg
aggttcgacagcgacgccgcgagtccgagagaggagccgcgggcgccgtggatagagcaggaggggccggagtattgggaccggaac
acacagatctacaaggcccaggcacagactgaccgagagagcctgcggaacctgcgcggctactacaaccagagcgaggccgggtct
cacaccctccagagcatgtacggctgcgacgtggggccggacgggcgcctcctccgcgggcatgaccagtacgcctacgacggcaag
gattacatcgccctgaacgaggacctgcgctcctggaccgccgcggacacggcggctcagatcacccagcgcaagtgggaggcggcc
cgtgaggcggagcagcggagagcctacctggagggcgagtgcgtggagtggctccgcagatacctggagaacgggaaggacaagctg
gagcgcgctgaccccccaaagacacacgtgacccaccaccccatctctgaccatgaggccaccctgaggtgctgggccctgggtttc
taccctgcggagatcacactgacctggcagcgggatggcgaggaccaaactcaggacactgagcttgtggagaccagaccagcagga
gatagaaccttccagaagtgggcagctgtggtggtgccttctggagaagagcagagatacacatgccatgtacagcatgaggggctg
ccgaagcccctcaccctgagatgggagccgtcttcccagtccaccgtccccatcgtgggcattgttgctggcctggctgtcctagca
gttgtggtcatcggagctgtggtcgctgctgtgatgtgtaggaggaagagttcaggtggaaaaggagggagctactctcaggctgcg
tgcagcgacagtgcccagggctctgatgtgtctctcacagcttga
Representative Human HLA-B*07:02 protein sequence
MLVMAPRTVLLLLSAALALTETWAGSHSMRYFYTSVSRPGRGEPRFISVGYVDDTQFVRFDSDAASPREEPRAPWIEQEGPEYWDRN
TQIYKAQAQTDRESLRNLRGYYNQSEAGSHTLQSMYGCDVGPDGRLLRGHDQYAYDGKDYIALNEDLRSWTAADTAAQITQRKWEAA
REAEQRRAYLEGECVEWLRRYLENGKDKLERADPPKTHVTHHPISDHEATLRCWALGFYPAEITLTWQRDGEDQTQDTELVETRPAG
DRTFQKWAAVVVPSGEEQRYTCHVQHEGLPKPLTLRWEPSSQSTVPIVGIVAGLAVLAVVVIGAVVAAVMCRRKSSGGKGGSYSQAA
CSDSAQGSDVSLTA
Representative Human HLA-A*24:02 DNA sequence
atggccgtcatggcgccccgaaccctcgtcctgctactctcgggggccctggccctgacccagacctgggcaggctcccactccatg
aggtatttctccacatccgtgtcccggcccggccgcggggagccccgcttcatcgccgtgggctacgtggacgacacgcagttcgtg
cggttcgacagcgacgccgcgagccagaggatggagccgcgggcgccgtggatagagcaggaggggccggagtattgggacgaggag
acagggaaagtgaaggcccactcacagactgaccgagagaacctgcggatcgcgctccgctactacaaccagagcgaggccggttct
cacaccctccagatgatgtttggctgcgacgtggggtcggacgggcgcttcctccgcgggtaccaccagtacgcctacgacggcaag
gattacatcgccctgaaagaggacctgcgctcttggaccgcggcggacatggcagctcagatcaccaagcgcaagtgggaggcggcc
catgtggcggagcagcagagagcctacctggagggcacgtgcgtggacgggctccgcagatacctggagaacgggaaggagacgctg
cagcgcacggacccccccaagacacatatgacccaccaccccatctctgaccatgaggccactctgagatgctgggccctgggcttc
taccctgcggagatcacactgacctggcagcgggatggggaggaccagacccaggacacggagcttgtggagaccaggcctgcaggg
gatggaaccttccagaagtgggcagctgtggtggtaccttctggagaggagcagagatacacctgccatgtgcagcatgagggtctg
cccaagcccctcaccctgagatgggagccatcttcccagcccaccgtccccatcgtgggcatcattgctggcctggttctccttgga
gctgtgatcactggagctgtggtcgctgctgtgatgtggaggaggaacagctcagatagaaaaggagggagctactctcaggctgca
agcagtgacagtgcccagggctctgatgtgtctctcacagcttgtaaagtgtga
Representative Human HLA-A*24:02 protein sequence
MAVMAPRTLVLLLSGALALTQTWAGSHSMRYFSTSVSRPGRGEPRFIAVGYVDDTQFVRFDSDAASQRMEPRAPWIEQEGPEYWDEE
TGKVKAHSQTDRENLRIALRYYNQSEAGSHTLQMMFGCDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEATLRCWALGFYPAEITLTWQRDGEDQTQDTELVETRPAG
DGTFQKWAAVVVPSGEEQRYTCHVQHEGLPKPLTLRWEPSSQPTVPIVGIIAGLVLLGAVITGAVVAAVMWRRNSSDRKGGSYSQAA
SSDSAQGSDVSLTACKV

Included in Tables 1-3 are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequences listed in Tables 1-3, or a portion thereof. Such polypeptides may have a function of the full-length peptide or polypeptide as described further herein.
Included in Tables 2 and 3 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed in Tables 2 or 3, or a portion thereof. Such nucleic acid molecules can have a function of the full-length nucleic acid as described further herein.

In some embodiments, the binding proteins provided herein comprise a constant region that is chimeric, humanized, human, primate, or rodent (e.g., rat or mouse). For example, a human variable region may be chimerized with a murine constant region or a murine variable region may be humanized with a human constant region and/or human framework regions. In some embodiments, the constant regions may be mutated to modify functionality (e.g., introduction of non-naturally occurring cysteine substitutions in opposing residue locations in TCR alpha and beta chains to provide disulfide bonds useful for increasing affinity between the TCR alpha and beta chains). Similarly, mutations may be made in the transmembrane domain of the constant region to modify functionality (e.g., to increase hydrophobicity by introducing a non-naturally occurring substitution of a residue with a hydrophobic amino acid). In some embodiments, each CDR of the binding protein has up to five amino acid substitutions, insertions, deletions, or a combination thereof as compared to a reference CDR sequence. In some embodiments, mutations may be made to the constant region to increase cell surface expression.

In some embodiments, the binding proteins disclosed herein may be engineered protein scaffolds, an antibody or an antigen-binding fragment thereof, TCR-mimic antibodies, and the like. Such binding moieties may be designed and/or generated against peptides and/or MHC-peptide complexes described herein using routine immunological methods, such as immunizing a host, obtaining antibody-producing cells and/or antibodies thereof, and generating hybridomas useful for producing monoclonal antibodies (e.g., Watt et al. (2006) Nat. Biotechnol. 24:177-183; Gebauer and Skerra (2009) Curr. Opin. Chem Biol. 13:245-255; Skerra et al. (2008) FEBS J. 275:2677-2683; Nygren et al. (2008) FEBS J. 275:2668-2676; Dana et al. (2012) Exp. Rev. Mol. Med. 14:e6; Sergeva et al. (2011) Blood 117:4262-4272; PCT Publ. Nos. WO 2007/143104, PCT/US86/02269, and WO 86/01533; U.S. Pat. No. 4,816,567; Better et al. (1988) Science 240:1041-1043; Liu et al. (1987) Proc. Natl. Acad. Sci. U.S.A. 84:3439-3443; Liu et al. (1987) J. Immunol. 139:3521-3526; Sun et al. (1987) Proc. Nat. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. If desired, binding moieties may be isolated or purified using conventional procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography, lectin chromatography, and high performance liquid chromatography (HPLC) (e.g., Current Protocols in Immunology, or Current Protocols in Protein Science, John Wiley & Sons, NY, N.Y.).

The terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin V_{L} domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publ. Nos. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically and/or selectively bind to an antigen (e.g., a peptide and/or an MHC-peptide complex described herein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, protein subunit peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically and/or selectively or substantially specifically and/or selectively to a peptide and/or an MHC-peptide complex described herein. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Similar to other binding moieties described herein, antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro/ex vivo or by somatic mutation in vivo), for example in the CDRs. The term “humanized antibody”, as used herein, also includes antibodies in which CDR sequences derived from the germline of another mammalian species, have been grafted onto human framework sequences.

In some embodiments, the binding proteins disclosed herein may comprise a T cell receptor (TCR), an antigen-binding fragment of a TCR, or a chimeric antigen receptor (CAR). In some embodiments, the binding protein disclosed herein may comprise two polypeptide chains, each of which comprises a variable region comprising a CDR3 of a TCR alpha chain and a CDR3 of a TCR beta chain, or a CDR1, CDR2, and CDR3 of both a TCR alpha chain and a TCR beta chain. In some embodiments, a binding protein comprises a single chain TCR (scTCR), which comprises both the TCR V_{α} and TCR V_{β} domains, but only a single TCR constant domain (C_{α} or C_{β}). The term “chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs encompassed by the present invention may include an extracellular portion comprising an antigen-binding domain (i.e., obtained or derived from an immunoglobulin or immunoglobulin-like molecule, such as an antibody or TCR, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al. (2013) Cancer Discov. 3:388, Harris and Kranz (2016) Trends Pharmacol. Sci. 37:220, and Stone et al. (2014) Cancer Immunol. Immunother. 63:1163).

In some embodiments, 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2.

In some embodiments, the binding proteins (e.g., the TCR, antigen-binding fragment of a TCR, or chimeric antigen receptor (CAR)) disclosed herein is chimeric (e.g., comprises amino acid residues or motifs from more than one donor or species), humanized (e.g., comprises residues from a non-human organism that are altered or substituted so as to reduce the risk of immunogenicity in a human), or human.

Methods for producing engineered binding proteins, such as TCRs, CARs, and antigen-binding fragments thereof, are well-known in the art (e.g., Bowerman et al. (2009) Mol. Immunol. 5:3000; U.S. Pat. Nos. 6,410,319; 7,446,191; U.S. Pat. Publ. No. 2010/065818; U.S. Pat. No. 8,822,647; PCT Publ. No. WO 2014/031687; U.S. Pat. No. 7,514,537; and Brentjens et al. (2007) Clin. Cancer Res. 73:5426).

In some embodiments, the binding protein described herein is a TCR, or antigen-binding fragment thereof, expressed on a cell surface, wherein the cell surface-expressed TCR is capable of more efficiently associating with a CD3 protein as compared to endogenous TCR A binding protein encompassed by the present invention, such as a TCR, when expressed on the surface of a cell like a T cell, may also have higher surface expression on the cell as compared to an endogenous binding protein, such as an endogenous TCR In some embodiments, provided herein is a CAR, wherein the binding domain of the CAR comprises an antigen-specific TCR binding domain (see, e.g., Walseng et al. (2017) Scientific Reports 7:10713).

Also provided are modified binding proteins (e.g., TCRs, antigen-binding fragments of TCRs, or CARs) that may be prepared according to well-known methods using a binding protein having one or more of the V_{α} and/or V_{β} sequences disclosed herein as starting material to engineer a modified binding protein that may have altered properties from the starting binding protein. A binding protein may be engineered by modifying one or more residues within one or both variable regions (i.e., V_{α} and/or V_{β}), for example within one or more CDR regions and/or within one or more framework regions. Additionally or alternatively, a binding protein may be engineered by modifying residues within the constant region(s).

Another type of variable region modification is to mutate amino acid residues within the V_{α} and/or V_{β} CDR1, CDR2 and/or CDR3 regions to thereby improve one or more binding properties (e.g., affinity) of the binding protein of interest. Site-directed mutagenesis or PCR-mediated mutagenesis may be performed to introduce the mutation(s) and the effect on protein binding, or other functional property of interest, may be evaluated in in vitro, ex vivo, or in vivo assays as described herein and provided in the Examples. In some embodiments, conservative modifications (as discussed above) may be introduced. The mutations may be amino acid substitutions, additions or deletions. In some embodiments, the mutations are substitutions. Moreover, typically no more than one, two, three, four or five residues within a CDR region are modified.

In some embodiments, binding proteins (e.g., TCRs, antigen-binding fragments of TCRs, or CARs) described herein may possess one or more amino acid substitutions, deletions, or additions relative to a naturally occurring TCR In some embodiments, each CDR of the binding protein has up to five amino acid substitutions, insertions, deletions, or a combination thereof as compared to the cognate reference CDR sequence listed in Table 2. Conservative substitutions of amino acids are well-known and may occur naturally or may be introduced when the binding protein is recombinantly produced. Amino acid substitutions, deletions, and additions may be introduced into a protein using mutagenesis methods known in the art (see, e.g., Sambrook et al. (2001) Molecular Cloning: A Laboratory Manual, 3d ed., Cold Spring Harbor Laboratory Press, NY). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al. supra).

A variety of criteria known to the ordinarily skilled artisan indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In some embodiments, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS™, Align, the BLAST algorithm, or other algorithms described herein and practiced in the art).

In some embodiments, an encoded binding protein (e.g., TCR, antigen-binding fragment of a TCR, or CAR) may comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In some embodiments, a binding protein (e.g., TCR, antigen-binding fragment of a TCR, or CAR) described herein comprises a mature V_{α} domain, a mature V_{β} domain, or both. In some embodiments, a binding protein (e.g., TCR, antigen-binding fragment of a TCR, or CAR) described herein comprises a mature TCR β-chain, a mature TCR α-chain, or both.

In some embodiments, the binding proteins are fusion proteins comprising: (a) an extracellular component comprising a TCR or antigen-binding fragment thereof, (b) an intracellular component comprising an effector domain or a functional portion thereof, and (c) a transmembrane domain connecting the extracellular and intracellular components. In some embodiments, the fusion protein is capable of binding (e.g., specifically and/or selectively) to a peptide-MHC (pMHC) complex comprising a MAGEC2 immunogenic peptide in the context of an MHC molecule (e.g., an MHC class I molecule).

As used herein, an “effector domain” or “immune effector domain” is an intracellular portion or domain of a fusion protein or receptor that can directly or indirectly promote an immune response in a cell when receiving an appropriate signal. In some embodiments, an effector domain is from an immune cell protein or portion thereof or immune cell protein complex that receives a signal when bound (e.g., CD3ζ), or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an intracellular tyrosine-based activation motif (ITAM), such as those found in costimulatory molecules. Without wishing to be bound by theory, it is believed that ITAMs are useful for T cell activation following ligand engagement by a T cell receptor or by a fusion protein comprising a T cell effector domain. In some embodiments, the intracellular component or functional portion thereof comprises an ITAM. Exemplary immune effector domains include but are not limited to those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMFI, Slp76, pTα, TCRα, TCRβ, TRIM, Zap70, PTCH2, or any combination thereof. In some embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that binds (e.g., specifically and/or selectively) with CD83, or a functional variant thereof, or any combination thereof. In some embodiments, the intracellular component comprises a CD28 costimulatory domain or a functional portion or variant thereof (which may optionally include a LL-GG mutation at positions 186-187 of the native CD28 protein (e.g., Nguyen et al. (2003) Blood 702:4320), a 4-1BB costimulatory domain or a functional portion or variant thereof, or both.

In some embodiments, an effector domain comprises a CD3s endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component encompassed by the present invention are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In some embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In some embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker encompassed by the present invention can position the binding domain away from the surface of a host cell expressing the fusion protein to enable proper contact between the host cell and a target cell, antigen binding, and activation (Patel et al. (1999) Gene Therapy 6:412-419). Linker length may be varied to maximize antigen recognition based on the selected target molecule, selected binding epitope, or antigen binding domain seize and affinity (see, e.g., Guest et al. (2005) Immunother. 28:203-11 and PCT Publ. No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂, (Gly₃Ser)₂, Gly₂Ser, or a combination thereof, such as ((Gly₃Ser)₂Gly₂Ser)).

A binding protein may be conjugated to an agent, such as a detection moiety, readiosensitizer, photosensitizer, and the like, and/or may be chemically modified as described above regarding peptides.

In any of the herein disclosed embodiments, the encoded binding protein is capable of bind to a peptide-MHC (pMHC) complex comprising a MAGEC2 immunogenic peptide in the context of an MHC molecule (e.g., an MHC class I molecule).

A variety of assays are well-known for assessing binding affinity and/or determining whether a binding molecule binds (e.g., specifically and/or selectively) to a particular ligand (e.g., peptide antigen-MHC complex). It is within the level of a skilled artisan to determine the binding affinity of a binding protein for a target, such as a T cell peptide epitope of a target polypeptide, such as by using any of a number of binding assays that are well-known in the art. For example, in some embodiments, a Biacore™ machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore™) analysis (see, e.g., Scatchard et al. (1949) Ann. N. Y. Acad. Sci. 51:660, Wilson (2002) Science 295:2103, Wolff et al. (1993) Cancer Res. 53:2560, and U.S. Pat. Nos. 5,283,173 and 5,468,614), flow cytometry, sequencing and other methods for detection of expressed nucleic acids. In one example, apparent affinity for a target is measured by assessing binding to various concentrations of tetramers, for example, by flow cytometry using labeled multimers, such as MHC-antigen tetramers. In one representative example, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

### VI. Nucleic Acids and Vectors

In an aspect encompassed by the present invention, provided herein are nucleic acid molecules that encode proteins described herein, such as MAGEC2 immunogenic peptides and fragments thereof, MHC molecules, binding proteins (e.g., TCRs, antigen-binding fragments of the TCRs, CARs, and the like), and the like.

In some embodiments, the nucleic acid molecule hybridizes, under stringent conditions, with the complement of a sequence with at least about at least about 80%, 810%, 82%, 83%, 84%, 85%, 86%, 87%, 88%, 89%, 90%, 91%, 92%, 93%, 94%, 95%, 96%, 97%, 98%, 99%, or more identity, such as over the full length, to a nucleic acid encoding a polypeptide selected from the group consisting of the polypeptide sequences listed in Tables 1-3.

In some embodiments, the nucleic acid molecule hybridizes, under stringent conditions, with the complement of a nucleic acid encoding a polypeptide selected from the group consisting of polypeptide sequences listed in Tables 1-3.

In some embodiments, the nucleic acid molecule comprises (e.g., comprises, consists essentially of, or consists of) a nucleotide sequence encoding a polypeptide selected from the group consisting of polypeptide sequences listed in Tables 1-3.

In some embodiments, the nucleic acid sequence encodes a MAGEC2 immunogenic peptides described herein.

In some embodiments, the nucleic acids comprise (e.g., comprise, consist essentially of, or consist of) a nucleotide sequence encoding at least one (e.g., one, two, or three) TCR α-chain CDR set forth in Table 2. In some embodiments, the nucleic acids comprise (e.g., comprise, consist essentially of, or consist of) a nucleotide sequence encoding a TCR V_{α} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{α} domain sequence set forth in Table 2. In some embodiments, the nucleic acids comprise (e.g., comprise, consist essentially of, or consist of) a nucleotide sequence encoding a TCR α-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR α-chain sequence set forth in Table 2.

In some embodiments, the nucleic acids comprise (e.g., comprise, consist essentially of, or consist of) a nucleotide sequence encoding at least one (e.g., one, two, or three) TCR β-chain CDR set forth in Table 2. In some embodiments, the nucleic acids comprise (e.g., comprise, consist essentially of, or consist of) a nucleotide sequence encoding a TCR V_{β} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{β} domain sequence set forth in Table 2. In some embodiments, the nucleic acids comprise (e.g., comprise, consist essentially of, or consist of) a nucleotide sequence encoding a TCR β-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR β-chain sequence set forth in Table 2.

The term “nucleic acid” includes “polynucleotide,” “oligonucleotide,” and “nucleic acid molecule,” and generally means a polymer of DNA or RNA, which may be single-stranded or double-stranded, synthesized or obtained (e.g., isolated and/or purified) from natural sources, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA).

In some embodiments, the nucleic acids encompassed by the present invention are recombinant. As used herein, the term “recombinant” refers to (i) molecules that are constructed outside living cells by joining natural or synthetic nucleic acid segments to nucleic acid molecules that may replicate in a living cell, or (ii) molecules that result from the replication of those described in (i) above. For purposes herein, the replication may be in vitro, ex vivo, or in vivo replication.

The nucleic acids can be constructed based on chemical synthesis and/or enzymatic ligation reactions using procedures known in the art. See, for example, Green and Sambrook et al. supra. For example, a nucleic acid may be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that may be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids encompassed by the present invention can be purchased from companies, such as Integrated DNA Technologies (Coralville, IA).

In one embodiment, the nucleic acid comprises a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In some embodiments, the nucleotide sequences described herein are codon-optimized for expression in a host cell (e.g., an immune cell, such as a T cell).

The present invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically and/or selectively hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions may be rendered more stringent by the addition of increasing amounts of formamide.

The present invention also provides a nucleic acid comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids described herein.

Typically, said nucleic acid is a DNA or RNA molecule, which may be included in a suitable vector, such as a plasmid, cosmid, episome, artificial chromosome, phage or a viral vector.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Thus, a further object encompassed by the present invention relates to a vector comprising a nucleic acid encompassed by the present invention.

Such vectors may comprise regulatory elements, such as a promoter, enhancer, terminator and the like, to cause or direct expression of said polypeptide upon administration to a subject Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell may be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, lentiviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses are well-known in the art and may be found, for instance, in PCT Publ. WO 95/14785, PCT. Publ. WO 96/22378, U.S. Pat. Nos. 5,882,877, 6,013,516, 4,861,719, 5,278,056, and PCT Publ. WO 94/19478.

In some embodiments, the composition comprises an expression vector comprising an open reading frame encoding a binding protein or a polypeptide described herein or a fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the binding protein, polypeptide or fragment thereof.

In some embodiments, the vector further comprises nucleic acid sequence encoding CD8α and/or CD8β. In certain embodiments, the nucleic acid sequence encoding CD8α or CD8β is operably linked to a nucleic acid encoding a tag (e.g., a CD34 enrichment tag). In specific embodiments, the nucleic acid sequence encoding CD8α and/or CD8β are interconnected with an internal ribosome entry site or a nucleic acid sequence encoding a self-cleaving peptide, such as P2A, E2A, F2A or T2A, etc.

In some embodiments, the expression vector provided herein comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids set forth in Tables 1-3.

As described above, representative examples of promoters include, but are not limited to, promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described herein. In some embodiments, enhancers/promoters include, for example, human actin, human myosin, human hemoglobin, human muscle creatine and viral enhancers such as those from CMV, RSV and EBV.

In some embodiments, the nucleic acid may be operably incorporated in a carrier or delivery vector as described further below. Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses. For example, a lentivirus vector may be used to infect T cells.

In some embodiments, the recombinant expression vector is capable of delivering a polynucleotide to an appropriate host cell, for example, a T cell or an antigen-presenting cell, i.e., a cell that displays a peptide/MHC complex on its cell surface (e.g., a dendritic cell) and lacks CD8. In some embodiments, the host cell is a hematopoietic progenitor cell or a human immune system cell. For example, the immune system cell may be a CD4⁺ T cell, a CD8⁺ T cell, a CD4/CD8 double negative T cell, a gd T cell, a natural killer cell, a dendritic cell, or any combination thereof. In some embodiments, wherein a T cell is the host, the T cell may be naive, a central memory T cell, an effector memory T cell, or any combination thereof. The recombinant expression vectors may therefore also include, for example, lymphoid tissue-specific transcriptional regulatory elements (TREs), such as a B lymphocyte, T lymphocyte, or dendritic cell specific TREs. Lymphoid tissue specific TREs are known in the art (see, e.g., Thompson et al. (1992) Mol. Cell. Biol. 72:1043, Todd et al. (1993) J. Exp. Med. 777:1663, and Penix et al. (1993) J. Exp. Med. 775:1483).

In some embodiments, a recombinant expression vector comprises a nucleotide sequence encoding a TCR α chain, a TCR β chain, and/or a linker peptide. For example, in some embodiments, the recombinant expression vector comprises a nucleotide sequence encoding the full-length TCR alpha and TCR beta chains of the binding protein with a linker positioned between them, wherein the nucleotide sequence encoding the beta chain is positioned 5′ of the nucleotide sequence encoding the alpha chain. In some embodiments, the nucleotide sequence encodes the full-length TCR alpha and TCR beta chains with a linker positioned between them, wherein the nucleotide sequence encoding the TCR beta chain is positioned 3′ of the nucleotide sequence encoding the TCR alpha chain. In some embodiments, the full-length TCR alpha and/or TCR beta chains are replaced with fragments thereof.

As described further below, another aspect encompassed by the present invention relates to a cell which has been transfected, infected or transformed by a nucleic acid and/or a vector in accordance with the present invention. A host cell may include any individual cell or cell culture which may receive a vector or the incorporation of nucleic acids and/or proteins, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods (see, e.g., Sambrook et al. (1989) Molecular Cloning: A Laboratory Manual 2d ed. (Cold Spring Harbor Laboratory)). The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids encompassed by the present invention may be used to produce a recombinant polypeptide encompassed by the present invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al (1980), rat YB2/3HL.P2.G11.16Ag.20 cell (ATCC CRL 1662, hereinafter referred to as “YB2/0 cell”), and the like. In some embodiments, the YB2/0 cell is used since ADCC activity of chimeric or humanized binding proteins is enhanced when expressed in this cell.

The present invention also encompasses methods of producing a recombinant host cell expressing binding proteins, peptides and fragments thereof encompassed by the present invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express said binding proteins, peptides and fragments thereof. Such recombinant host cells may be used for the diagnostic, prognostic, and/or therapeutic method encompassed by the present invention.

In another aspect, as described above, the present invention provides isolated nucleic acids that hybridize under selective hybridization conditions to a polynucleotide disclosed herein. Thus, the polynucleotides of this embodiment may be used for isolating, detecting, and/or quantifying nucleic acids comprising such polynucleotides. For example, polynucleotides encompassed by the present invention may be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. In some embodiments, the cDNA library comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, full-length sequences. The cDNA libraries may be normalized to increase the representation of rare sequences. Low or moderate stringency hybridization conditions are typically, but not exclusively, employed with sequences having a reduced sequence identity relative to complementary sequences. Moderate and high stringency conditions may optionally be employed for sequences of greater identity. Low stringency conditions allow selective hybridization of sequences having about 70% sequence identity and may be employed to identify orthologous or paralogous sequences. Optionally, polynucleotides encompassed by the present invention will encode at least a portion of a binding protein encoded by the polynucleotides described herein. The polynucleotides encompassed by the present invention embrace nucleic acid sequences that may be employed for selective hybridization to a polynucleotide encoding a binding protein encompassed by the present invention (see, e.g., Ausubel, supra and Colligan, supra).

### VII. Engineered Cells

In an aspect encompassed by the present invention, provided herein are host cells that express proteins described herein, such as MAGEC2 immunogenic peptides, MAGEC2 immunogenic peptide-MHC (pMHC) complexes, MAGEC2 binding proteins (e.g., TCRs, antigen-binding fragments of TCRs, CARs, or fusion proteins comprising a TCR and an effector domain), and the like described herein. In some embodiments, the host cells comprise the nucleic acids or vectors described herein.

In some embodiments, a polynucleotide encoding a binding protein is used to transform, transfect, or transduce a host cell (e.g., a T cell) for use in adoptive transfer therapy. Advances in nucleic acid sequencing and particular TCR sequencing have been described (e.g., Robins et al. (2009) Blood 114:4099; Robins et al. (2010) Sci. Translat. Med. 2:47ra64, Robins et al. (2011) J. Imm. Meth., and Warren et al. (2011) Genome Res. 21:790) and may be employed in the course of practicing embodiments encompassed by the present invention. Similarly, methods for transfecting or transducing T cells with desired nucleic acids are well-known in the art (e.g., U.S. Pat. Publ. No. US 2004/0087025) as have adoptive transfer procedures using T cells of desired antigen-specificity (e.g., Schmitt et al. (2009) Hum. Gen. 20:1240, Dossett et al. (2009) Mol. Ther. 77:742, Till et al. (2008) Blood 772:2261, Wang et al. (2007) Hum. Gene Ther. 18:112, Kuball et al. (2007) Blood 709:2331, U.S. Pat. Publ. 2011/0243972, U.S. Pat. Publ. 2011/0189141, and Leen et al. (2007) Ann. Rev. Immunol. 25:243).

Any suitable immune cell may be modified to include a heterologous polynucleotide encompassed by the present invention, including, for example, a T cell, a NK cell, or a NK-T cell. In some embodiments, the cell may be a primary cell or a cell of a cell line. In some embodiments, a modified immune cell comprises a CD4⁺ T cell, a CD8⁺ T cell, or both. For purposes herein, the T cell may be any T cell, such as a cultured T cell, e.g., a primary T cell, or a T cell from a cultured T cell line, e.g., Jurkat, SupTl, etc., or a T cell obtained from a mammal. If obtained from a mammal, the T cell may be obtained from numerous sources, including but not limited to blood, bone marrow, lymph node, the thymus, or other tissues or fluids. T cells may also be enriched for or purified. In some embodiments, the T cell is a human T cell. In some embodiments, the T cell is a T cell isolated from a human. The T cell may be any type of T cell and may be of any developmental stage, including but not limited to, cytotoxic lymphocyte, cytotoxic lymphocyte precursor cell, cytotoxic lymphocyte progenitor cell, cytotoxic lymphocyte stem cell, CD4+/CD8+ double positive T cells, CD4+ helper T cells, e.g., Th1 and Th2 cells, CD4⁺ T cells, CD8⁺ T cells (e.g., cytotoxic T cells), tumor infiltrating lymphocytes (TILs), memory T cells (e.g., central memory T cells and effector memory T cells), naive T cells, and the like.

Any appropriate method may be used to transfect or transduce the cells (e.g., T cells), or to administer the nucleotide sequences or compositions encompassed by methods described herein. Methods for delivering polynucleotides to host cells include, for example, use of cationic polymers, lipid-like molecules, and certain commercial products such as, for example, in vivo-jetPEI®. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

Modified immune cells as described herein may be functionally characterized using methodologies for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions.

Procedures for performing these and similar assays may be found, for example, in Lefkovits (Immunology Methods Manual: Hie Comprehensive Sourcebook of Techniques, 1998), as well as Current Protocols in Immunology, Weir, (1986) Handbook of Experimental Immunology, Blackwell Scientific, Boston, MA; Mishell and Shigii (eds.) (1979) Selected Methods in Cellular Immunology, Freeman Publishing, San Francisco, CA; Green and Reed (1998) Science 281:1309, and references cited therein.

In some embodiments, apparent affinity for a binding protein, such as a TCR or antigen-binding portion thereof, may be measured by assessing binding to various concentrations of MHC multimers. “MHC-peptide multimer staining” refers to an assay used to detect antigen-specific T cells, which, in some embodiments, features a tetramer of MHC molecules, each comprising an identical peptide having an amino acid sequence that is cognate (e.g., identical or related to) at least one antigen (e.g., a MAGEC2 immunogenic peptide), wherein the complex is capable of binding to a binding protein, such as a TCR or antigen-binding portion thereof, that recognizes the cognate antigen. Each of the MHC molecules may be tagged with a biotin molecule. Biotinylated MHC/peptides may be multimerized (e.g., tetramerized) by the addition of streptavidin, which may be fluorescently labeled.

The multimer may be detected by flow cytometry via the fluorescent label. In some embodiments, a pMHC multimer assay is used to detect or select enhanced affinity binding protein, such as a TCR or antigen-binding portion thereof, encompassed by the present invention. In some examples, apparent K_{D} of a binding protein, such as a TCR or antigen-binding portion thereof, is measured using 2-fold dilutions of labeled multimers at a range of concentrations, followed by determination of binding curves by non-linear regression, apparent K_{D} being determined as the concentration of ligand that yielded half-maximal binding.

Levels of cytokines may be determined using methods described herein, such as ELISA, ELISPOT, intracellular cytokine staining, and flow cytometry and combinations thereof (e.g., intracellular cytokine staining and flow cytometry).

Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Thl immune response and a Th2 immune response may be examined, for example, by determining levels of Thl cytokines, such as IFN-g, IL-12, IL-2, and TNF-b, and Type 2 cytokines, such as IL-4, IL-5, IL-9, IL-10, and IL-13.

A host cell encompassed by the present invention may comprise a single polynucleotide that encodes a binding protein as described herein, or the binding protein may be encoded by more than one polynucleotide. In other words, components or portions of a binding protein may be encoded by two or more polynucleotides, which may be contained on a single nucleic acid molecule or may be contained on two or more nucleic acid molecules.

In some embodiments, a polynucleotide encoding two or more components or portions of a binding protein encompassed by the present invention comprises the two or more coding sequences operatively associated in a single open reading frame. Such an arrangement can advantageously allow coordinated expression of desired gene products, such as, for example, contemporaneous expression of alpha- and beta-chains of a TCR, such that they are produced in about a 1:1 ratio. In some embodiments, two or more substituent gene products of a binding protein encompassed by the present invention, such as a TCR (e.g., alpha- and beta-chains) or CAR, are expressed as separate molecules and associate post-translationally. In further embodiments, two or more substituent gene products of a binding protein encompassed by the present invention are expressed as a single peptide with the parts separated by a cleavable or removable segment. For instance, self-cleaving peptides useful for expression of separable polypeptides encoded by a single polynucleotide or vector are known in the art and include, for example, a porcine teschovirus-1 2 A (P2A) peptide, a thoseaasigna virus 2A (T2A) peptide, an equine rhinitis A virus (ERAV) 2A (E2A) peptide, and a foot-and-mouth disease vims 2A (F2A) peptide.

In some embodiments, a binding protein encompassed by the present invention comprises one or more junction amino acids. “Junction amino acids” or “junction amino acid residues” refer to one or more (e.g., 2 to about 10) amino acid residues between two adjacent motifs, regions or domains of a polypeptide, such as between a binding domain and an adjacent constant domain or between a TCR chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein encompassed by the present invention (e.g., a P2A peptide disposed between a TCR α-chain and a TCR β-chain, the self-cleavage of which can leave one or more junction amino acids in the α-chain, the TCR β-chain, or both).

Engineered immune cells encompassed by the present invention may be administered as therapies for, e.g., a disorder characterized by MAGEC2 expression. In some circumstances, it may be desirable to reduce or stop the activity associated with a cellular immunotherapy. Thus, in some embodiments, an engineered immune cell encompassed by the present invention comprises a heterologous polynucleotide encoding a binding protein and an accessory protein, such as a safety switch protein, which can be targeted using a cognate drug or other compound to selectively modulate the activity (e.g., lessen or ablate) of such cells when desirable. Safety switch proteins used in this regard include, for example, a truncated EGF receptor polypeptide (huEGFRt) that is devoid of extracellular N-terminal ligand binding domains and intracellular receptor tyrosine kinase activity but retains the native amino acid sequence, type I transmembrane cell surface localization, and a conformationally intact binding epitope for pharmaceutical-grade anti-EGFR monoclonal antibody, cetuximab (Erbitux) tEGF receptor (tEGFr; Wang et al. (2011) Blood 118:1255-1263), a caspase polypeptide (e.g., iCasp9; Straathof et al. (2005) Blood 105:4247-4254, Di Stasi et al. (2011) N. Engl. J. Med. 365:1673-1683, Zhou and Brenner (2016) Hematol. pii:S0301-472X:30513-30516), RQR8 (Philip et al. (2014) Blood 124:1277-1287), and a human c-myc protein tag (Kieback et al. (2008) Proc. Natl. Acad. Sci. USA 105:623-628).

Other accessory components useful for therapeutic cells comprise a tag or selection marker (e.g., a CD34 enrichment tag) that allows the cells to be identified, sorted, isolated, enriched, or tracked. For example, marked immune cells having desired characteristics (e.g., an antigen-specific TCR and a safety switch protein) may be sorted away from unmarked cells in a sample and more efficiently activated and expanded for inclusion in a therapeutic product of desired purity.

As used herein, the term “selection marker” comprises a nucleic acid construct that confers an identifiable change to a cell permitting detection and positive selection of immune cells transduced with a polynucleotide comprising a selection marker. For example, RQR is a selection marker that comprises a major extracellular loop of CD20 and two minimal CD34 binding sites. In some embodiments, an RQR-encoding polynucleotide comprises a polynucleotide that encodes the 16 amino acid CD34 minimal epitope. In some embodiments, such as certain embodiments provided in the examples herein, the CD34 minimal epitope is incorporated at the amino terminal position of the CD8 stalk domain (Q8). In further embodiments, the CD34 minimal binding site sequence may be combined with a target epitope for CD20 to form a compact marker/suicide gene for T cells (RQR8) (Philip et al. 2014). This construct allows for the selection of immune cells expressing the construct, with for example, CD34-specific antibody bound to magnetic beads (Miltenyi) and that utilizes clinically accepted pharmaceutical antibody, rituximab, that allows for the selective deletion of a transgene expressing engineered T cell (e.g., Philip et al. (2014) Blood 124:1277-1287, U.S. Pat. Publ. 2015-0093401, and U.S. Pat. Publ. 2018-0051089).

Further exemplary selection markers include several truncated type I transmembrane proteins normally not expressed on T cells: the truncated low-affinity nerve growth factor, truncated CD19, and truncated CD34 (e.g., Di Stasi et al. (2011) N. Engl. J. Med. 365:1673-1683, Mavilio et al. (1994) Blood 83:1988-1997, and Fehse et al. (2000) Mol. Ther. 7:448-456). A particularly attractive feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non-signaling domains or various proteins (e.g., CD19, CD34, LNGFR, etc.) also may be employed. Any selection marker may be employed and should be acceptable for good manufacturing practices. In some embodiments, selection markers are expressed with a polynucleotide that encodes a gene product of interest (e.g., a binding protein encompassed by the present invention, such as a TCR or CAR, or antigen-binding fragment thereof). Further examples of selection markers include, for example, reporters such as GFP, EGFP, β-gal or chloramphenicol acetyltransferase (CAT). In some embodiments, a selection marker, such as, for example, CD34 is expressed by a cell and the CD34 may be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34.

In some embodiments, a selection marker comprises an RQR polypeptide, a truncated low-affinity nerve growth factor (tNGFR), a truncated CD19 (tCD19), a truncated CD34 (tCD34), or any combination thereof.

By way of background, inclusion of CD4⁺ T cells in an immunotherapy cell product can provide antigen-induced IL-2 secretion and augment persistence and function of transferred cytotoxic CD8⁺ T cells (e.g., Kennedy et al. (2008) Immunol. Rev. 222:129 and Nakanishi et al. Nature (2009) 52:510). In some embodiments, a class I-restricted TCR in CD4⁺ T cells may require the transfer of a CD8 co-receptor to enhance sensitivity of the TCR to class I HLA peptide complexes. CD4 co-receptors differ in structure to CD8 and cannot effectively substitute for CD8 co-receptors (e.g., Stone & Kranz (2013) Front. Immunol. 4:244 and Cole et al. (2012) Immunology 737:139). Thus, another accessory protein for use in the compositions and methods encompassed by the present invention comprises a CD8 co-receptor or component thereof. Engineered immune cells comprising a heterologous polynucleotide encoding a binding protein encompassed by the present invention may, in some embodiments, further comprise a heterologous polynucleotide encoding a CD8 co-receptor protein, or a beta-chain or alpha-chain component thereof.

A host cell may be efficiently transduced to contain, and may efficiently express, a single polynucleotide that encodes the binding protein, safety switch protein, selection marker, and CD8 co-receptor protein.

In one embodiment, the host cell encompassed by the present invention further includes a nucleic acid encoding a co-stimulatory molecule, such that the modified T cell expresses the co-stimulatory molecule. In some embodiments, the co-stimulatory domain is selected from CD3, CD27, CD28, CD83, CD86, CD127, 4-1BB, 4-1BBL, PD1 and PD1L.

In any of the foregoing embodiments, a host cell that express the binding protein described herein may be a universal immune cell. A “universal immune cell” comprises an immune cell that has been modified to reduce or eliminate expression of one or more endogenous genes that encode a polypeptide product selected from PD-1, LAG-3, CTLA4, TIM3, TIGIT, an HLA molecule, a TCR molecule, or any combination thereof. Without wishing to be bound by theory, certain endogenously expressed immune cell proteins may downregulate the immune activity of the modified immune cells (e.g., PD-1, LAG-3, CTLA4, TIGIT), or may interfere with the binding activity of a heterologously expressed binding protein encompassed by the present invention (e.g., an endogenous TCR that binds a non-MAGEC2 antigen and interferes with the modified immune cell binding to a target cell that expresses a MAGEC2 antigen such as a MAGEC2 immunogenic peptide in the context of an MHC molecule. Further, endogenous proteins (e.g., immune cell proteins, such as an HLA allele) expressed on a donor immune cell may be recognized as foreign by an allogeneic host, which may result in elimination or suppression of the modified donor immune cell by the allogeneic host

Accordingly, decreasing or eliminating expression or activity of such endogenous genes or proteins can improve the activity, tolerance, or persistence of the modified immune cells in an autologous or allogeneic host setting, and allows universal administration of the cells (e.g., to any recipient regardless of HLA type). In some embodiments, cells in accordance with the present invention are syngeneic, meaning that they are genetically identical or sufficiently identical and immunologically compatible as to allow for transplantation. In some embodiments, a universal immune cell is a donor cell (e.g., allogeneic) or an autologous cell. In some embodiments, a modified immune cell (e.g., a universal immune cell) encompassed by the present invention comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al. (2016) Nature Sci. Rep. 6:21757; Torikai et al. (2012) Blood 179:5697; and Torikai et al. (2013) Blood 722:1341, which also provide representative, exemplary gene editing techniques, compositions, and adoptive cell therapies useful according to the present invention).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product Alterations resulting in a chromosomal gene knockout may include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In some embodiments, a chromosomal gene knock-out or gene knock-in may be made by chromosomal editing of a host cell. Chromosomal editing may be performed using, for example, endonucleases. As used herein “endonuclease” refers to an enzyme capable of catalyzing cleavage of a phosphodiester bond within a polynucleotide chain. In some embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues may be changed to alter triplet sequence specificity (e.g., Desjarlais et al. (1993) Proc. Natl. Acad. Sci. 90:2256-2260 and Wolfe et al. (1999) J. Mol. Biol. 255:1917-1934). Multiple zinc finger motifs may be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In some embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the repeat variable diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also well-known in the art (e.g., U.S. Pat. Publ. No. US 2011/0301073, which atypical RVDs are incorporated by reference herein in their entirety). TALENs may be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In some embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences may be introduced at the site of DSB via homology directed repair. The crRNA and tracrRNA may be engineered into a single guide RNA (sgRNA or gRNA) (e.g., Jinek et al. (2012) Science 337:816-821). Further, the region of the guide RNA complementary to the target site may be altered or programed to target a desired sequence (Xie et al. (2014) PLOS One 9:e100448, U.S. Pat Publ. No. US 2014/0068797, U.S. Pat Publ. No. US 2014/0186843, U.S. Pat. No. 8,697,359, and PCT Publ. No. WO 2015/071474). In some embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al. (2017) Clin. Cancer Res. 23:2255-2266, which provides representative, exemplary gRNAs, CAS9 DNAs, vectors, and gene knockout techniques.

As used herein, a “meganuclease,” also referred to as a “homing endonuclease,” refers to an endodeoxyribonuclease characterized by a large recognition site (double stranded DNA sequences of about 12 to about 40 base pairs). Meganucleases may be divided into five families based on sequence and structure motifs: LAGLIDADG, GlY-YIG, HNH, His-Cys box, and PD-(D/E)XK. Exemplary meganucleases include I-Scel, I-Ceul, PI-PspI, RI-Sce, I-ScelV, I-Csml, I-Panl, I-Scell, I-Ppol, I-SceIII, I-Crel, I-Tevl, I-TevII and I-TevIII, whose recognition sequences are well-known (e.g., U.S. Pat. Nos. 5,420,032 and 6,833,252, Belfort et al. (1997) Nucl. Acids Res. 25:3379-3388, Dujon et al. (1989) Gene 52:115-118, Perler et al. (1994) Nucl. Acids Res. 22:1125-1127, Jasin (1996) Trends Genet. 72:224-228, Gimble et al. (1996) J. Mol. Biol. 263:163-180, and Argast et al. (1998) J. Mol. Biol. 280:345-353).

In some embodiments, naturally-occurring meganucleases may be used to promote site-specific genome modification of a target of interest, such as an immune checkpoint, an HLA-encoding gene, or a TCR component-encoding gene.

In other embodiments, an engineered meganuclease having a novel binding specificity for a target gene is used for site-specific genome modification (see, e.g., Porteus et al. (2005) Nat. Biotechnol. 23:967-73, Sussman et al. (2004) J. Mol. Biol. 342:31-41, Epinat et al. (2003) Nucl. Acids Res. 37:2952-2962, Chevalier et al. (2002) Mol. Cell 70:895-905, Ashworth et al. (2006) Nature 441:656-659, Paques et al. (2007) Curr. Gene Ther. 7:49-66, and U.S. Pat. Publ. Nos. US 2007/0117128, US 2006/0206949, US 2006/0153826, US 2006/0078552, and US 2004/0002092). In further embodiments, a chromosomal gene knockout is generated using a homing endonuclease that has been modified with modular DNA binding domains of TALENs to make a fusion protein known as a megaTAL. MegaTALs may be utilized to not only knock-out one or more target genes, but to also introduce (knock in) heterologous or exogenous polynucleotides when used in combination with an exogenous donor template encoding a polypeptide of interest

In some embodiments, a chromosomal gene knockout comprises an inhibitory nucleic acid molecule that is introduced into a host cell (e.g., an immune cell) comprising a heterologous polynucleotide encoding an antigen-specific receptor that binds (e.g., specifically and/or selectively) to a MAGEC2 antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host immune cell.

A chromosomal gene knockout may be confirmed directly by DNA sequencing of the host immune cell following use of the knockout procedure or agent.

Chromosomal gene knockouts may also be inferred from the absence of gene expression (e.g., the absence of an mRNA or polypeptide product encoded by the gene) following the knockout.

In some embodiments, a host cell encompassed by the present invention is capable of specifically and/or selectively killing 50% or more of target cells that comprise a peptide-MHC (pMHC) complex comprising a MAGEC2 immunogenic peptide in the context of an MHC molecule.

In some embodiments, the modified immune cell is capable of producing a cytokine when contacted with target cells that comprise a peptide-MHC (pMHC) complex comprising a MAGEC2 immunogenic peptide in the context of an MHC molecule.

In some embodiments, the cytokine comprises IFN-γ or IL2. In some embodiments, the cytokine comprises TNF-α.

In some embodiments, the host cell is capable of producing a higher level of cytokine or a cytotoxic molecule when contacted with a target cell with expression of MAGEC2 at a level of less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low MAGEC2 expression level is termed “heterozygous expression” meaning between about 1 TPM and about 35 TPM, or any range in between, inclusive, such as 1-32 TPM. For example, the host cell is capable of producing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher level of cytokine or a cytotoxic molecule.

In some embodiments, the host cell is capable of specifically and/or selectively killing a target cell expressing MAGEC2 (e.g., a hyperproliferative cell expressing MAGEC2). In certain embodiments, the target cell expresses a MAGEC2 immunogenic peptide in the context of an MHC molecule (e.g., a matched MHC molecule).

In some embodiments, host cells do not express MAGEC2 antigen, are not recognized by a binding protein described herein, are not of serotype HLA-B*07, and/or do not express an HLA-B*07 allele, such as HLA-B*0702, HLA-B*0704, HLA-B*0705, HLA-B*0709, HLA-B*0710, HLA-B*0715, or HLA-B*0721 allele. For example, a patient may receive host cells from a healthy donor who is MAGEC2-negative or negative for an MHC that presents a MAGEC2 immunogenic peptide described herein (such as HLA-B*07:02-negative and/or HLA-A*24:02-negative), or even autologous cells that have selected and/or engineered. Cells isolated from that donor may be used as the source of transplant material. In parallel, T cells isolated from the same donor may be genetically engineered to recognize MAGEC2, such as by expressing a MAGEC2 binding protein described herein. Donor cells may be used to engraft cell populations into the patient (e.g., hematopoietic stem cells used to reconstitute an immune system) and host cells may be infused into the patient with the goal of eliciting a highly specific anti-tumor effect. The engineered donor T cells may be designed to recognize and eliminate the patient's MAGEC2-positive, thereby preventing relapse and promoting complete cures of disorders characterized by MAGEC2 expression. Because the transplanted cells are derived from the donor and are therefore either MAGEC2-negative, serotype negative for an MHC that presents a MAGEC2 immunogenic peptide described herein (such as HLA-B*07 serotype negative and/or HLA-A*24:02 serotype negative, and/or negative for an MHC that presents a MAGEC2 immunogenic peptide described herein (such as HLA-B*07:02-negative and/or HLA-A*24:02-negative), engineered cells described herein may have minimal toxic side effects. Such patient-matched host cells and treatment methods may be used according to therapeutic methods described further below.

In some embodiments, the killing is determined by a killing assay. In some embodiment, the killing assay is carried out by coculturing the host cell and the target cell at a ratio from 20:1 to 0.625:1, for example, from 15:1 to 1.25:1, from 10:1 to 1.5:1, from 8:1 to 3:1, from 6:1 to 5:1, 20:1 to 5:1, 10:1 to 2.5:1 etc. In some embodiments, the target cell is pulsed with 1 μg/mL to 50 μg/mL of MAGEC2 peptide, for example, from 1 ug/mL to 10 ng/mL, 500 ng/mL to 0.5 ng/mL, from 10 ng/mL to 10 μg/mL from 250 ng/mL to 1 ng/mL, from 50 ng/mL to 5 ng/mL, from 20 ng/mL to 10 ng/mL, etc.

In some embodiments, the host cell is capable of killing a higher number of target cells when contacted with target cells with a level of MAGEC2 less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 73 TPM). For example, the host cell may be capable of killing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher number of target cells.

The present invention further provides a population of cells comprising at least one host cell described herein. The population of cells may be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells may be a substantially homogeneous population, in which the population comprises mainly of host cells (e.g., consisting essentially of) comprising the recombinant expression vector. The population also may be a clonal population of cells, in which all cells of the population are clones of a single host cell comprising a recombinant expression vector, such that all cells of the population comprise the recombinant expression vector. In one embodiment encompassed by the present invention, the population of cells is a clonal population comprising host cells comprising a recombinant expression vector as described herein.

In an embodiment encompassed by the present invention, the numbers of cells in the population may be rapidly expanded. Expansion of the numbers of T cells may be accomplished by any of a number of methods as are well-known in the art (e.g., U.S. Pat. Nos. 8,034,334 and 8,383,099, U.S. Pat. Publ. No. 2012/0244133, Dudley et al. (2003) J. Immunother. 26:332-242, and Riddell et al. (1990) J. Immunol. Methods 128:189-201). For example, expansion of the numbers of T cells may be carried out by culturing the T cells with OKT3 antibody, IL-2, and feeder PBMC (e.g., irradiated allogeneic PBMC).

### VIII. Pharmaceutical Compositions

In another aspect encompassed by the present invention, pharmaceutical compositions are provided herein comprising compositions described herein (e.g., binding proteins, nucleic acids, cells, and the like) and a pharmaceutically acceptable carrier, diluent, or excipient.

The term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Agents and other compositions encompassed by the present invention may be specially formulated for administration in solid or liquid form, including those adapted for various routes of administration, such as (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. Any appropriate form factor for an agent or composition described herein, such as, but not limited to, tablets, capsules, liquid syrups, soft gels, suppositories, and enemas, is contemplated.

Pharmaceutical compositions encompassed by the present invention may be presented as discrete dosage forms, such as capsules, sachets, or tablets, or liquids or aerosol sprays each containing a pre-determined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms may be prepared by any of the methods of pharmacy.

Suitable excipients include water, saline, dextrose, glycerol, or the like and combinations thereof. In some embodiments, compositions comprising host cells, binding proteins, or fusion proteins as disclosed herein further comprise a suitable infusion media. Suitable infusion media may be any isotonic medium formulation, typically normal saline, Normosol™-R (Abbott) or Plasma-Lyte™ A (Baxter), 5% dextrose in water, Ringer's lactate may be utilized. An infusion medium may be supplemented with human serum albumin or other human serum components. Unit doses comprising an effective amount of a host cell, or composition are also contemplated.

Also provided herein are unit doses that comprise an effective amount of a host cell or of a composition comprising the host cell. As described herein, host cells include immune cells, T cells (CD4⁺ T cells and/or CD8⁺ T cells), cytotoxic lymphocytes (e.g., cytotoxic T cells and/or natural killer (NK) cells), and the like. For example, in some embodiments, a unit dose comprises a composition comprising at least about 30%, at least about 40%, at least about 50%, at least about 60%), at least about 70%, at least about 80%, at least about 85%, at least about 90%, or at least about 95% engineered cells, either alone or in combination with other cells, such as comprising at least about 30%, at least about 40%, at least about 50%, at least about 60%), at least about 70%, at least about 80%, at least about 85%, at least about 90%, or at least about 95% other cells. In some embodiments, undesired cells are present at a reduced amount or substantially not present, such as less than about 50%, less than about 40%, less than about 30%, less than about 20%, less than about 10%, less than about 5%, or less then about 1% the population of cells in the composition.

The amount of cells in a composition or unit dose is at least one cell (for example, at least one engineered CD8⁺ T cell, engineered CD4⁺ T cell, and/or NK cell) or is more typically greater than 10² cells, for example, up to 10⁶, up to 10⁷, up to 10⁸ cells, up to 10⁹ cells, or more than 10¹⁰ cells. In some embodiments, the cells are administered in a range from about 10⁶ to about 10¹⁰ cells/m², such as in a range of about 10⁵ to about 10⁹ cells/m².

The number of cells will depend upon the ultimate use for which the composition is intended as well the type of cells included therein. For example, cells modified to contain a binding protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 ml or less, 250 ml or less, or 100 ml or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells may be apportioned into multiple infusions that cumulatively equal or exceed 10⁶, 10⁷, 10⁸, 10⁹, 10¹⁰, or 10¹¹ cells. In some embodiments, a unit dose of the engineered immune cells may be co-administered with (e.g., simultaneously or contemporaneously) hematopoietic stem cells from an allogeneic donor.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutically effective amount” may be used in reference to treatment, whereas “prophylactically effective amount” may be used to describe administrating an effective amount to a subject that is susceptible or at risk of developing a disease or disease-state (e.g., recurrence) as a preventative course.

The pharmaceutical compositions described herein may be presented in unit-dose or multi-dose containers, such as sealed ampoules or vials. Such containers may be frozen to preserve the stability of the formulation until infusion into the patient. In some embodiments, a unit dose comprises a host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polyethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In some embodiments, the pharmaceutical composition described herein and as described above for immunogenic compositions representatively exemplified for peptides, when administered to a subject, can elicit an immune response against a cell of interest that expresses MAGEC2. Such pharmaceutical compositions may be useful as vaccines for prophylactic and/or therapeutic treatment of a disorder characterized by MAGEC2 expression.

### IX. Uses and Methods

The compositions described herein may be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In some uses and methods encompassed by the present invention, subjects or subject samples are utilized. In some embodiments, the subject is an animal. The animal may be of either sex and may be at any stage of development. In some embodiments, the animals is a vertebrate, such as a mammal. In some embodiments, the subject is a non-human mammal. In some embodiments, the subject is a domesticated animal, such as a dog, cat, cow, pig, horse, sheep, or goat. In some embodiments, the subject is a companion animal, such as a dog or cat. In some embodiments, the subject is a livestock animal, such as a cow, pig, horse, sheep, or goat. In some embodiments, the subject is a zoo animal. In some embodiments, the subject is a research animal, such as a rodent (e.g., mouse or rat), dog, pig, or non-human primate. In some embodiments, the animal is a genetically engineered animal. In some embodiments, the animal is a transgenic animal (e.g., transgenic mice and transgenic pigs). In some embodiments, the subject is a fish or reptile.

In some embodiments, the subject is a rodent, such as a mouse. In some such embodiments, the mouse is a transgenic mouse, such as a mouse expressing human MHC (i.e., HLA) molecules, such as HLA-B72 (e.g., Nicholson et al. (2012) Adv. Hematol. 2012:404081). In some embodiments, the subject is a transgenic mouse expressing human TCRs or is an antigen-negative mouse (e.g., Li et al. (2010) Nat. Med. 16:1029-1034 and Obenaus et al. (2015) Nat. Biotechnol. 33:402-407). In some embodiments, the subject is a transgenic mouse expressing human HLA molecules and human TCRs. In some embodiments, such as where the subject is a transgenic HLA mouse, the identified TCRs are modified, e.g., to be chimeric or humanized. In some embodiments, the TCR scaffold is modified, such as analogous to known binding protein humanizing methods.

In some embodiments, the subject is a human. In some embodiments, the subject is an animal model of a disorder characterized by MAGEC2 expression, such as a cancer. For example, the animal model may be an orthotopic xenograft animal model of a human-derived cancer.

In some embodiments, the subject is a human, such as a human with a disorder characterized by MAGEC2 expression.

The methods described herein may be used to treat a subject in need thereof. As used herein, a “subject in need thereof” includes any subject who has a disorder characterized by MAGEC2 expression, a relapse of a disorder characterized by MAGEC2 expression, and/or who is predisposed to a disorder characterized by MAGEC2 expression.

In some embodiments of the methods encompassed by the present invention, the subject has not undergone treatment for a disorder characterized by MAGEC2 expression, such as chemotherapy, radiation therapy, targeted therapy, and/or immunotherapies. In some embodiments, the subject has undergone treatment for a disorder characterized by MAGEC2 expression, such as chemotherapy, radiation therapy, targeted therapy, and/or immunotherapies.

In some embodiments, the subject has had surgery to remove cancerous or precancerous tissue. In some embodiments, the cancerous tissue has not been removed, e.g., the cancerous tissue may be located in an inoperable region of the body, such as in a tissue that is essential for life, or in a region where a surgical procedure would cause considerable risk of harm to the patient.

In some embodiments, the subject or cells thereof are resistant to a therapy of relevance, such as resistant to standard of care therapy, immune checkpoint inhibitor therapy, and the like. For example, modulating one or more biomarkers encompassed by the present invention may overcome resistance to immune checkpoint inhibitor therapy.

In some embodiments, the subjects are in need of modulation according to compositions and methods described herein, such as having been identified as having an unwanted absence, presence, or aberrant MAGEC2 expression.

a. Diagnostic Methods

In an aspect encompassed by the present invention, provided herein are diagnostic methods for detecting the presence or absence of a MAGEC2 antigen, a MAGEC2 antigen-MHC complex, a cell of interest expressing MAGEC2, and/or a cell having had exposure to MAGEC2, comprising detecting the presence or absence of said MAGEC2 antigen in a sample by use of at least one binding protein, or at least one host cell described herein. In some embodiments, the method further comprising obtaining the sample (e.g., from a subject). In some embodiments, the at least one binding protein or the at least one host cell, forms a complex with a MAGEC2 peptide epitope in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

In an aspect encompassed by the present invention, provided herein are diagnostic methods for detecting the level of MAGEC2 or a disorder characterized by MAGEC2 expression in a subject, comprising: a) contacting a sample obtained from the subject with at least one agent (e.g., a MAGEC2 immunogenic peptide, MAGEC2 immunogenic peptide-MHC complex (pMHC), binding protein, host cell, or a population of host cells described herein); and b) detecting the level of reactivity, wherein a higher level of reactivity compared to a control level indicates the level of a disorder characterized by MAGEC2 expression in the subject.

In some embodiments, the level of reactivity is indicated by T cell activation or effector function, such as, but not limited to, T cell proliferation, killing, or cytokine release. The control level may be a reference number or a level of a healthy subject who has no exposure to a disorder characterized by MAGEC2 expression.

A biological sample may be obtained from a subject for determining the presence and level of an immune response to the agent as described herein. A “biological sample” as used herein may be a blood sample (from which serum or plasma may be prepared), biopsy specimen, body fluids (e.g., blood, isolated PBMCs, isolated T cells, lung lavage, ascites, mucosal washings, synovial fluid, etc.), bone marrow, lymph nodes, tissue explant, organ culture, or any other tissue or cell preparation from the subject or a biological source. Biological samples may also be obtained from the subject prior to receiving any pharmaceutical composition, which biological sample is useful as a control for establishing baseline data.

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

The level of an immune response, such as a cytotoxic T lymphocyte (CTL), may be determined by any one of numerous immunological methods described herein and routinely practiced in the art. For example, the level of a CTL immune response may be determined prior to and following administration of any one of the herein described binding proteins expressed by, for example, a T cell. Cytotoxicity assays for determining CTL activity may be performed using any one of several techniques and methods routinely practiced in the art (e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

The present invention provides, in part, methods, systems, and code for accurately classifying whether a biological sample is associated with an output of interest, such as expression of a target of interest, such as MAGEC2. In some embodiments, the present invention is useful for classifying a sample (e.g., from a subject) as associated with or at risk for responding to or not responding to therapy for a disorder associated with MAGEC2 expression using a statistical algorithm and/or empirical data.

An exemplary method for detecting the amount or activity of MAGEC2, and thus useful for classifying whether a sample is likely or unlikely to respond to a therapy for a disorder associated with MAGEC2 expression involves contacting a biological sample with an agent, such as a MAGEC2 immunogenic peptide or binding agent described herein, capable of detecting the amount or activity of MAGEC2 in the biological sample. In some embodiments, the method further comprise obtaining a biological sample, such as from a test subject. In some embodiments, at least one agent is used, wherein two, three, four, five, six, seven, eight, nine, ten, or more such agents may be used in combination (e.g., in sandwich ELISAs) or in serial. In certain instances, the statistical algorithm is a single learning statistical classifier system. For example, a single learning statistical classifier system may be used to classify a sample as a based upon a prediction or probability value and the presence or level of the biomarker. The use of a single learning statistical classifier system typically classifies the sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In some embodiments, the diagnosis of a subject is followed by administering to the individual a therapeutically effective amount of a defined treatment based upon the diagnosis.

In some embodiments, the methods further involve obtaining a control biological sample (e.g., biological sample from a subject who does not have a disorder associated with MAGEC2 expression, a subject who is in remission, a subject whose disorder is susceptible to therapy, a subject whose disorder is progressing, or other subjects of interest).

In some embodiments of analytical methods described herein, MAGEC2 expression (e.g., in a sample from a subject) is compared to a pre-determined control (standard) sample. The sample from the subject is typically from a diseased tissue, such as cancer cells or tissues. The control sample may be from the same subject or from a different subject. The control sample is typically a normal, non-diseased sample. However, in some embodiments, such as for staging of disease or for evaluating the efficacy of treatment, the control sample may be from a diseased tissue. The control sample may be a combination of samples from several different subjects. In some embodiments, the MAGEC2 expression measurement(s) from a subject is compared to a pre-determined level. This pre-determined level is typically obtained from normal samples. As described herein, a “pre-determined” expression may be used to, by way of example only, evaluate a subject that may be selected for treatment, evaluate a response to cancer, and/or evaluate a response to a combination cancer therapy. A pre-determined biomarker amount and/or activity measurement(s) may be determined in populations of patients with or without a disorder associated with MAGEC2 expression. The pre-determined biomarker amount and/or activity measurement(s) may be a single number, equally applicable to every patient, or the pre-determined biomarker amount and/or activity measurement(s) may vary according to specific sub-populations of patients. Age, weight, height, and other factors of a subject may affect the pre-determined biomarker amount and/or activity measurement(s) of the individual. Furthermore, the pre-determined biomarker amount and/or activity may be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis may be based on the ratio of pre-treatment biomarker measurement as compared to post-treatment biomarker measurement. Pre-treatment biomarker measurement may be made at any time prior to initiation of a therapy. Post-treatment biomarker measurement may be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring. Treatment may comprise therapy to treat the disorder characterized by MAGEC2 expression, either alone or in combination with other agents, such as anti-cancer agents like chemotherapy or immune checkpoint inhibitors.

The pre-determined MAGEC2 expression may be any suitable standard. For example, the pre-determined MAGEC2 expression may be obtained from the same or a different subject for whom a subject selection is being assessed. In one embodiment, the pre-determined biomarker amount and/or activity measurement(s) may be obtained from a previous assessment of the same patient. In such a manner, the progress of the selection of the patient may be monitored over time. In addition, the control may be obtained from an assessment of another human or multiple humans, e.g., selected groups of humans, if the subject is a human. In such a manner, the extent of the selection of the human for whom selection is being assessed may be compared to suitable other humans, e.g., other humans who are in a similar situation to the human of interest, such as those suffering from similar or the same condition(s) and/or of the same ethnic group.

In some embodiments encompassed by the present invention the change of MAGEC2 expression from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cut-off values apply equally when the measurement is based on relative changes, such as based on the ratio of pre-treatment biomarker measurement as compared to post-treatment biomarker measurement.

In some embodiments, MAGEC2 expression may be detected and/or quantified by detecting or quantifying MAGEC2 polypeptide or antigen thereof, such as by using a composition described herein. The polypeptide may be detected and quantified by any of a number of means well-known to those of skill in the art, such as by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991).

b. Therapeutic Methods

In an aspect encompassed by the present invention, provided herein are methods for preventing and/or treating a disorder characterized by MAGEC2 expression and/or for inducing an immune response against a cell of interest, such as a hyperproliferative cell, expressing MAGEC2. In some embodiments, the method comprises administering to a subject a therapeutically effective amount of a composition described herein, such as an immunogenic composition, cells expressing at least one binding protein, and the like. The methods encompassed by the present invention also may be used to determine the responsiveness to cancer therapy of many different cancers in subjects such as those described herein.

In some embodiments, the disorder characterized by MAGEC2 expression is a cancer. The terms “cancer” or “tumor” or “hyperproliferative” refer to the presence of cells possessing characteristics typical of cancer-causing cells, such as uncontrolled proliferation, immortality, invasive or metastatic potential, rapid growth, and certain characteristic morphological features. In some embodiments, such cells exhibit such characteristics in part or in full due to the expression and activity of immune checkpoint proteins, such as PD-1, PD-L1, PD-L2, and/or CTLA-4.

Cancer cells are often in the form of a tumor, but such cells may exist alone within an animal, or may be a non-tumorigenic cancer cell, such as in a hematologic cancer like leukemia. As used herein, the term “cancer” includes premalignant as well as malignant cancers. Cancers include, but are not limited to, a variety of cancers, carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, brain tumor, lung carcinoma (including lung adenocarcinoma), small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In some embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated. In some embodiments, the cancer is selected from the group consisting of (advanced) non-small cell lung cancer, melanoma, head and neck squamous cell cancer, (advanced) urothelial bladder cancer, (advanced) kidney cancer (RCC), microsatellite instability-high cancer, classical Hodgkin lymphoma, (advanced) gastric cancer, (advanced) cervical cancer, primary mediastinal B-cell lymphoma, (advanced) hepatocellular carcinoma, breast invasive carcinoma, bladder urothelial carcinoma, and (advanced) merkel cell carcinoma.

In addition, the compositions described herein may also be administered in combination therapy to further modulate a desired activity. Additional agents include, without limitations, chemotherapeutic agents, hormones, antiangiogens, radiolabelled, compounds, or with surgery, cryotherapy, and/or radiotherapy. The preceding treatment methods may be administered in conjunction with other forms of conventional therapy (e.g., standard-of-care treatments for cancer well-known to the skilled artisan), either consecutively with, pre- or post-conventional therapy. For example, these modulatory agents may be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and may be determined by the physician.

Therapy using one or more compositions described herein, either alone or in combination with other therapies, such as cancer therapies, may be used to contact MAGEC2-expressing cells and/or administered to a desired subject, such as a subject that is indicated as being a likely responder to therapy. In another embodiment, such therapy may be avoided once a subject is indicated as not being a likely responder to the therapy (e.g., as assessed according to a diagnostic or prognostic method described herein) and an alternative treatment regimen, such as targeted and/or untargeted cancer therapies, may be recommended and/or administered.

The term “targeted therapy” refers to administration of agents that selectively interact with a chosen biomolecule to thereby treat cancer. For example, targeted therapy regarding the inhibition of immune checkpoint inhibitor is useful in combination with the methods encompassed by the present invention.

The term “immunotherapy” or “immunotherapies” generally refers to any strategy for modulating an immune response in a beneficial manner and encompasses the treatment of a subject afflicted with, or at risk of contracting or suffering a recurrence of, a disease by a method comprising inducing, enhancing, suppressing or otherwise modifying an immune response, as well as any treatment that uses certain parts of a subject's immune system to fight diseases, such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” In some embodiments, an immunotherapy is specific for cells of interest, such as cancer cells. In some embodiments, immunotherapy may be “untargeted,” which refers to administration of agents that do not selectively interact with immune system cells, yet modulates immune system function. Representative examples of untargeted therapies include, without limitation, chemotherapy, gene therapy, and radiation therapy.

Some forms of immunotherapy are targeted therapies that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer. Similarly, immunotherapy may take the form of cell-based therapies. For example, adoptive cellular immunotherapy is a type of immunotherapy using immune cells, such as T cells, that have a natural or genetically engineered reactivity to a patient's cancer are generated and then transferred back into the cancer patient. The injection of a large number of activated tumor-specific T cells may induce complete and durable regression of cancers.

Immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer.

In some embodiments, an immunotherapeutic agent is an agonist of an immune-stimulatory molecule; an antagonist of an immune-inhibitory molecule; an antagonist of a chemokine; an agonist of a cytokine that stimulates T cell activation; an agent that antagonizes or inhibits a cytokine that inhibits T cell activation; and/or an agent that binds to a membrane bound protein of the B7 family. In some embodiments, the immunotherapeutic agent is an antagonist of an immune-inhibitory molecule. In some embodiments, the immunotherapeutic agents may be agents for cytokines, chemokines and growth factors, for examples, neutralizing antibodies that neutralize the inhibitory effect of tumor associated cytokines, chemokines, growth factors and other soluble factors, including IL-10, TGF-β and VEGF.

In some embodiments, immunotherapy comprises inhibitors of one or more immune checkpoints. The term “immune checkpoint” refers to a group of molecules on the cell surface of CD4+ and/or CD8⁺ T cells that fine-tune immune responses by modulating anti-cancer immune responses, such as down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD200R, CD160, gp49B, PIR-B, KRLG-1, KIR family receptors, TIM-1, TIM-3, TIM-4, LAG-3 (CD223), IDO, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragments, as well as nucleic acids encoding full-length immune checkpoint proteins.

Some immune checkpoints are “immune-inhibitory immune checkpoints” encompassing molecules (e.g., proteins) that inhibit, down-regulate, or suppress a function of the immune system (e.g., an immune response). For example, PD-L1 (programmed death-ligand 1), also known as CD274 or B7-H1, is a protein that transmits an inhibitory signal that reduces proliferation of T cells to suppress the immune system. CTLA-4 (cytotoxic T-lymphocyte-associated protein 4), also known as CD152, is a protein receptor on the surface of antigen-presenting cells that serves as an immune checkpoint (“off” switch) to downregulate immune responses. TIM-3 (T-cell immunoglobulin and mucin-domain containing-3), also known as HAVCR2, is a cell surface protein that serves as an immune checkpoint to regulate macrophage activation. VISTA (V-domain Ig suppressor of T cell activation) is a type I transmembrane protein that functions as an immune checkpoint to inhibit T cell effector function and maintain peripheral tolerance. LAG-3 (lymphocyte-activation gene 3) is an immune checkpoint receptor that negatively regulates proliferation, activation, and homeostasis of T cells. BTLA (B- and T-lymphocyte attenuator) is a protein that displays T cell inhibition via interactions with tumor necrosis family receptors (TNF-R). KIR (killer-cell immunoglobulin-like receptor) is a family of proteins expressed on NK cells, and a minority of T cells, that suppress the cytotoxic activity of NK cells. In some embodiments, immunotherapeutic agents may be agents specific to immunosuppressive enzymes such as inhibitors that may block the activities of arginase (ARG) and indoleamine 2,3-dioxygenase (IDO), an immune checkpoint protein that suppresses T cells and NK cells, which change the catabolism of the amino acids arginine and tryptophan in the immunosuppressive tumor microenvironment. The inhibitors may include, but are not limited to, N-hydroxy-L-Arg (NOHA) targeting to ARG-expressing M2 macrophages, nitroaspirin or sildenafil (Viagra®), which blocks ARG and nitric oxide synthase (NOS) simultaneously; and IDO inhibitors, such as 1-methyl-tryptophan. The term further encompasses biologically active protein fragment, as well as nucleic acids encoding full-length immune checkpoint proteins and biologically active protein fragments thereof. In some embodiment, the term further encompasses any fragment according to homology descriptions provided herein.

By contrast, other immune checkpoints are “immune-stimulatory” encompassing molecules (e.g., proteins) that activate, stimulate, or promote a function of the immune system (e.g., an immune response). In some embodiments, the immune-stimulatory molecule is CD28, CD80 (B7.1), CD86 (B7.2), 4-1BB (CD137), 4-1BBL (CD137L), CD27, CD70, CD40, CD40L, CD122, CD226, CD30, CD30L, OX40, OX40L, HVEM, BTLA, GITR and its ligand GITRL, LIGHT, LTβR, LTαβ, ICOS (CD278), ICOSL (B7-H2), and NKG2D. CD40 (cluster of differentiation 40) is a costimulatory protein found on antigen presenting cells that is required for their activation. OX40, also known as tumor necrosis factor receptor superfamily member 4 (TNFRSF4) or CD134, is involved in maintenance of an immune response after activation by preventing T-cell death and subsequently increasing cytokine production. CD137 is a member of the tumor necrosis factor receptor (TNF-R) family that co-stimulates activated T cells to enhance proliferation and T cell survival. CD122 is a subunit of the interleukin-2 receptor (IL-2) protein, which promotes differentiation of immature T cells into regulatory, effector, or memory T cells. CD27 is a member of the tumor necrosis factor receptor superfamily and serves as a co-stimulatory immune checkpoint molecule. CD28 (cluster of differentiation 28) is a protein expressed on T cells that provides co-stimulatory signals required for T cell activation and survival. GITR (glucocorticoid-induced TNFR-related protein), also known as TNFRSF18 and AITR, is a protein that plays a key role in dominant immunological self-tolerance maintained by regulatory T cells. ICOS (inducible T-cell co-stimulator), also known as CD278, is a CD28-superfamily costimulatory molecule that is expressed on activated T cells and play a role in T cell signaling and immune responses.

Immune checkpoints and their sequences are well-known in the art and representative embodiments are described further below. Immune checkpoints generally relate to pairs of inhibitory receptors and the natural binding partners (e.g., ligands). For example, PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells. The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell inhibitory signals, and the ability to modulate the immune response. The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). The term “PD-1 ligand activity” includes the ability of a PD-1 ligand polypeptide to bind its natural receptor(s) (e.g., PD-1 or B7-1), the ability to modulate immune cell inhibitory signals, and the ability to modulate the immune response.

As used herein, the term “immune checkpoint therapy” refers to the use of agents that inhibit immune-inhibitory immune checkpoints, such as inhibiting their nucleic acids and/or proteins. Inhibition of one or more such immune checkpoints may block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that may either bind and/or inactivate or inhibit immune checkpoint proteins, or fragments thereof; as well as RNA interference, antisense, nucleic acid aptamers, etc. that may downregulate the expression and/or activity of immune checkpoint nucleic acids, or fragments thereof. Exemplary agents for upregulating an immune response include antibodies against one or more immune checkpoint proteins that block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents may directly block the interaction between the one or more immune checkpoints and its natural receptor(s) (e.g., antibodies) to prevent inhibitory signaling and upregulate an immune response. Alternatively, agents may indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain may binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. Therapeutic agents used for blocking the PD-1 pathway include antagonistic antibodies and soluble PD-L1 ligands. The antagonist agents against PD-1 and PD-L1/2 inhibitory pathway may include, but are not limited to, antagonistic antibodies to PD-1 or PD-L1/2 (e.g., 17D8, 2D3, 4H1, 5C4 (also known as nivolumab or BMS-936558), 4A11, 7D3 and 5F4 disclosed in U.S. Pat. No. 8,008,449; AMP-224, pidilizumab (CT-011), pembrolizumab, and antibodies disclosed in U.S. Pat. Nos. 8,779,105; 8,552,154; 8,217,149; 8,168,757; 8,008,449; 7,488,802; 7,943,743; 7,635,757; and 6,808,710. Similarly, additional representative checkpoint inhibitors may be, but are not limited to, antibodies against inhibitory regulator CTLA-4 (anti-cytotoxic T-lymphocyte antigen 4 anti-cytotoxic T-lymphocyte antigen 4), such as ipilimumab, tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and other antibodies, such as those disclosed in U.S. Pat. Nos. 8,748,815; 8,529,902; 8,318,916; 8,017,114; 7,744,875; 7,605,238; 7,465,446; 7,109,003; 7,132,281; 6,984,720; 6,682,736; 6,207,156; and 5,977,318, as well as EP Pat. No. 1212422, U.S. Pat Publ. Numbers 2002/0039581 and 2002/086014, and Hurwitz et al. (1998) Proc. Natl. Acad. Sci. U.S.A. 95:10067-10071.

The representative definitions of immune checkpoint activity, ligand, blockade, and the like exemplified for PD-1, PD-L1, PD-L2, and CTLA-4 apply generally to other immune checkpoints.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary agents include, but are not limited to, alkylating agents: nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Similarly, additional exemplary agents including platinum-containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AGO13736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE®)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard et. al. (2003) Exp. Hematol. 31:446-454); Herceg (2001) Mut. Res. 477:97-110). Poly(ADP-ribose) polymerase 1 (PARP1) is a key molecule in the repair of DNA single-strand breaks (SSBs) (de Murcia J. et al. (1997) Proc. Natl. Acad. Sci. U.S.A. 94:7303-7307; Schreiber et al. (2006) Nat. Rev. Mol. Cell Biol. 7:517-528; Wang et al. (1997) Genes Dev. 11:2347-2358). Knockout of SSB repair by inhibition of PARP1 function induces DNA double-strand breaks (DSBs) that may trigger synthetic lethality in cancer cells with defective homology-directed DSB repair (Bryant et al. (2005) Nature 434:913-917; Farmer et al. (2005) Nature 434:917-921). The foregoing examples of chemotherapeutic agents are illustrative and are not intended to be limiting.

In another embodiment, radiation therapy is used. The radiation used in radiation therapy may be ionizing radiation. Radiation therapy may also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal β-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy may be administered as external beam radiation or teletherapy wherein the radiation is directed from a remote source. The radiation treatment may also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments may comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy may be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It may be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, may cause discomfort or even significant local pain in about half the patients treated. It may also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents may kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT may be directed through a fiber-optic (a very thin glass strand). The fiber-optic is placed close to the cancer to deliver the proper amount of light. The fiber-optic may be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and may include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also may be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser may remove thin layers from the skin's surface without penetrating the deeper layers. This technique is particularly useful in treating tumors that have not spread deep into the skin and certain precancerous conditions. As an alternative to traditional scalpel surgery, the C02 laser is also able to cut the skin. The laser is used in this way to remove skin cancers. Neodymium:yttrium-aluminum-garnet (Nd:YAG) laser—Light from this laser may penetrate deeper into tissue than light from the other types of lasers, and it may cause blood to clot quickly. It may be carried through optical fibers to less accessible parts of the body. This type of laser is sometimes used to treat throat cancers. Argon laser—This laser may pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light may be directed to parts of the body without making a large incision. More procedures may be done on an outpatient basis. Lasers may be used in two ways to treat cancer: by shrinking or destroying a tumor with heat, or by activating a chemical—known as a photosensitizing agent—that destroys cancer cells. In PDT, a photosensitizing agent is retained in cancer cells and may be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers may be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems may produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

In one aspect, provided herein is a method of eliciting in a subject an immune response to a cell that expresses MAGEC2. In some embodiments, the method comprises administering to the subject a pharmaceutical composition described herein, wherein the pharmaceutical composition, when administered to the subject, elicits an immune response to the cell that expresses MAGEC2.

In some embodiments, the immune response can include a cell-mediated immune response. A cellular immune response is a response that involves T cells and may be determined in vitro, ex vivo, or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In another aspect encompassed by the present invention, the methods provided herein include administering to both human and non-human mammals as described above. Veterinary applications also are contemplated. In some embodiments, the subject may be any living organism in which an immune response may be elicited.

In some embodiments, the pharmaceutical composition may be administered at any time that is appropriate. For example, the administering may be conducted before or during treatment of a subject having a disorder characterized by MAGEC2 expression, and continued after the disorder characterized by MAGEC2 expression becomes clinically undetectable. The administering also may be continued in a subject showing signs of recurrence.

In some embodiments, the pharmaceutical composition may be administered in a therapeutically or a prophylactically effective amount Administering the pharmaceutical composition to the subject may be carried out using known procedures, and at dosages and for periods of time sufficient to achieve a desired effect.

In some embodiments, the pharmaceutical composition may be administered to the subject at any suitable site. Administration may be accomplished using methods generally known in the art. Agents, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine encompassed by the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

In some embodiments, the dose may be administered in an amount and for a period of time effective in bringing about a desired response, be it eliciting the immune response or the prophylactic or therapeutic treatment of a disorder characterized by MAGEC2 expression and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies may be provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient's peripheral blood lymphocyte (PBL) as responders and immunogenic peptides or peptide-MHC complexes described herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

In general, an appropriate dosage and treatment regimen provides the active molecules or cells in an amount sufficient to provide a benefit Such a response may be monitored by establishing an improved clinical outcome (e.g., more frequent remissions, complete or partial, or longer disease-free survival) in treated subjects as compared to non-treated subjects. Increases in preexisting immune responses to a viral protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro, ex vivo, and in vivo animal studies) and clinical studies and analyzing data obtained therefrom by appropriate statistical, biological, and clinical methods and techniques, all of which can readily be practiced by an ordinarily skilled artisan.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., engineered immune cells with one or more cytokines; immunosuppressive therapy such as calcineurin inhibitors, corticosteroids, microtubule inhibitors, low dose of a mycophenolic acid prodrug, or any combination thereof).

In some embodiments, a plurality of doses of a host cell (e.g., an engineered immune cell) described herein is administered to the subject, which may be administered at intervals between administrations of about two to about four weeks.

Treatment or prevention methods encompassed by the present invention may be administered to a subject as part of a treatment course or regimen, which may comprise additional treatments prior to, or after, administration of the instantly disclosed unit doses, cells, or compositions. For example, in some embodiments, a subject receiving a unit dose of the host cell (e.g., an engineered immune cell) is receiving or had previously received a hematopoietic cell transplant (HCT; including myeloablative and non-myeloablative HCT). In any of the foregoing embodiments, a hematopoietic cell used in an HCT may be a “universal donor” cell that is modified to reduce or eliminate expression of one or more endogenous genes that encode a polypeptide product selected from an MHC, antigen, and a binding protein (e.g., by a chromosomal gene knockout according to the methods described herein). In some embodiments,

Techniques and regimens for performing cell transplantation are known in the art and may comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in some embodiments, a host cell (e.g., an engineered immune cell) encompassed by the present invention may be administered with or shortly after stem cell therapy.

Methods encompassed by the present invention may, in some embodiments, further include administering one or more additional agents to treat the disease or disorder (e.g., a disorder characterized by MAGEC2 expression) in a combination therapy. For example, in some embodiments, a combination therapy comprises administering host cell or binding protein encompassed by the present invention with (concurrently, simultaneously, or sequentially) an antiviral agent. In some embodiments, a combination therapy comprises administering a host cell or binding protein encompassed by the present invention with lopinavir/ritonavir, chloroquine, ribavirin, steroid drugs, hydroxychloroquine, and/or interferon α. In some embodiments, a combination therapy comprises administering a host cell, composition, or unit dose of the host cells encompassed by the present invention with a secondary therapy, such as a surgery, an antibody, a vaccine, or any combination thereof.

c. Screening Methods

Another aspect encompassed by the present invention encompasses screening assays.

In some embodiments, methods are provided for selecting agents that bind to a MAGEC2 immunogenic peptide or pMHC described herein. For example, a method of identifying a peptide-binding molecule, or antigen-binding fragment thereof, that binds to a peptide epitope selected from the peptide sequences listed in Table 1 comprising a) providing a cell presenting a peptide epitope selected from the peptide sequences listed in Table 1 in the context of an MHC molecule on the surface of the cell; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

In some embodiments, a method of identifying a peptide-binding molecule or antigen-binding fragment thereof that binds to a peptide epitope selected from the peptide sequences listed in Table 1 comprising: a) providing a peptide epitope either alone or in a stable MHC-peptide complex, comprising a peptide epitope selected from the peptide sequences listed in Table 1, either alone or in the context of an MHC molecule; b) determining binding of a plurality of candidate peptide-binding molecules or antigen-binding fragments thereof to the peptide or stable MHC-peptide complex; and c) identifying one or more peptide-binding molecules or antigen-binding fragments thereof that bind to the peptide epitope or the stable MHC-peptide complex, optionally wherein the MHC or MHC-peptide complex is as described herein, is provided.

In some embodiments, provide herein are methods of identifying a peptide-binding molecule or antigen-binding fragment thereof that binds to a peptide epitope selected from Table 1.

In some embodiments, the peptide binding molecule (e.g., MHC-peptide binding molecule) is a molecule or portion thereof that possesses the ability to bind (e.g., specifically and/or selectively) to a peptide epitope that is presented or displayed in the context of an MHC molecule (MHC-peptide complex), such as on the surface of a cell. Exemplary peptide binding molecules include T cell receptors or antibodies, or antigen-binding portions thereof, including single chain immunoglobulin variable regions (e.g., scTCR, scFv) thereof, that exhibit specific ability to bind to an MHC-peptide complex. In some embodiments, the peptide binding molecule is a TCR or antigen-binding fragment thereof. In some embodiments, the peptide binding molecule is an antibody, such as a TCR-like antibody or antigen-binding fragment thereof. In some embodiments, the peptide binding molecule is a TCR-like CAR that contains an antibody or antigen binding fragment thereof, such as a TCR-like antibody, such as one that has been engineered to bind to MHC-peptide complexes. In some embodiments, the peptide binding molecule may be derived from natural sources, or it may be partly or wholly synthetically or recombinantly produced.

In some embodiments, a binding molecule that binds to a peptide epitope may be identified by contacting one or more candidate peptide binding molecules, such as one or more candidate TCR molecules, antibodies, or antigen-binding fragments thereof, with an MHC-peptide complex, and assessing whether each of the one or more candidate binding molecules binds (e.g., specifically and/or selectively) to the MHC-peptide complex. The methods may be performed in vitro, ex vivo, or in vivo. Methods are well-known in the art for screening, such as described in U.S. Pat. Publ. 2020/0102553.

In some embodiments, the methods include contacting a plurality or library of binding molecules, such as a plurality or library of TCRs or antibodies, with an MHC-restricted epitope and identifying or selecting molecules that specifically and/or selectively bind such an epitope. In some embodiments, a library or collection containing a plurality of different binding molecules, such as a plurality of different TCRs or a plurality of different antibodies, may be screened or assessed for binding to an MHC-restricted epitope. In some embodiments, such as for selecting a binding protein that specifically and/or selectively binds an MHC-restricted peptide, hybridoma methods may be employed.

In some embodiments, screening methods may be employed in which a plurality of candidate binding molecules, such as a library or collection of candidate binding molecules, are individually contacted with an peptide binding molecule, either simultaneously or sequentially. Library members that specifically and/or selectively bind to a particular MHC-peptide complex may be identified or selected. In some embodiments, the library or collection of candidate binding molecules may contain at least 2, 5, 10, 100, 10³, 10⁴, 10⁵, 10⁶, 10⁷, 10⁸, 10⁹, or more different peptide binding molecules.

In some embodiments, the methods may be employed to identify a peptide binding molecule, such as a TCR or an antibody, that exhibits binding for more than one MHC haplotype or more than one MHC allele. In some embodiments, the peptide binding molecule, such as a TCR or antibody, specifically and/or selectively binds or recognizes a peptide epitope presented in the context of a plurality of MHC class I haplotypes or alleles. In some embodiments, the peptide binding molecule, such as a TCR or antibody, specifically and/or selectively binds or recognizes a peptide epitope presented in the context of a plurality of MHC class II haplotypes or alleles.

A variety of assays are known for assessing binding affinity and/or determining whether a binding molecule specifically and/or selectively binds to a particular ligand (e.g., MHC-peptide complex). It is within the level of a skilled artisan to determine the binding affinity of a TCR for a T cell epitope of a target polypeptide, such as by using any of a number of binding assays that are well-known in the art. For example, in some embodiments, a Biacore® machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore®) analysis (see, e.g., Scatchard et al. (1949) Ann. N.Y. Acad. Sci. 51:660; Wilson (2002) Science 295:2103; Wolff et al. (1993) Cancer Res. 53:2560; and U.S. Pat. Nos. 5,283,173, 5,468,614, or the equivalent), flow cytometry, sequencing and other methods for detection of expressed nucleic acids. In one example, apparent affinity for a TCR is measured by assessing binding to various concentrations of tetramers, for example, by flow cytometry using labeled tetramers. In one example, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

In some embodiments, the methods may be used to identify binding molecules that bind only if the particular peptide is present in the complex, and not if the particular peptide is absent or if another, non-overlapping or unrelated peptide is present. In some embodiments, the binding molecule does not substantially bind the MHC in the absence of the bound peptide, and/or does not substantially bind the peptide in the absence of the MHC. In some embodiments, the binding molecules are at least partially specific. In some embodiments, an exemplary identified binding molecule may bind to an MHC-peptide complex if the particular peptide is present, and also bind if a related peptide that has one or two substitutions relative to the particular peptide is present.

In some embodiments, an identified antibody, such as a TCR-like antibody, may be used to produce or generate a chimeric antigen receptors (CARs) containing a non-TCR antibody that specifically and/or selectively binds to a MHC-peptide complex.

In some embodiments, the methods of identifying a peptide binding molecule, such as a TCR or TCR-like antibody or TCR-like CAR, may be used to engineer cells expressing or containing a peptide binding molecule. In some embodiments, a cell or engineered cell is a T cell. In some embodiments, the T cell is a CD4+ or CD8+ T cell. In some embodiments, the peptide binding molecule recognizes a MHC class I peptide complex, an MHC class II peptide complex and/or an MHC-E peptide complex. In some embodiments, a peptide binding molecule, such as a TCR or antibody or CAR, that specifically and/or selectively recognizes a peptide in the context of an MHC class I may be used to engineer CD8⁺ T cells. In some embodiments, also provided is a composition of engineered CD8+ T cells expressing or containing the TCR, antibody or CAR, for recognition of a peptide presented in the context of MHC class I. In any of such embodiments, the cells may be used in methods of adoptive cell therapy.

In some embodiments, TCR libraries may be generated by amplification of the repertoire of Vα and Vβ from T cells isolated from a subject, including cells present in PBMCs, spleen or other lymphoid organ. In some cases, T cells may be amplified from tumor-infiltrating lymphocytes (TILs). In some embodiments, TCR libraries may be generated from CD4+ or CD8+ cells. In some embodiments, the TCRs may be amplified from a T cell source of a normal of healthy subject, i.e., normal TCR libraries. In some embodiments, the TCRs may be amplified from a T cell source of a diseased subject, i.e., diseased TCR libraries. In some embodiments, degenerate primers are used to amplify the gene repertoire of Vα and Vβ, such as by RT-PCR in samples, such as T cells, obtained from humans. In some embodiments, scTv libraries may be assembled from naive Vα and Vβ libraries in which the amplified products are cloned or assembled to be separated by a linker. Depending on the source of the subject and cells, the libraries may be HLA allele-specific.

Alternatively, in some embodiments, TCR libraries may be generated by mutagenesis or diversification of a parent or scaffold TCR molecule. For example, in some aspects, a subject, e.g., human or other mammal such as a rodent, may be vaccinated with a peptide, such as a peptide identified by the present methods. In some embodiments, a sample may be obtained from the subject, such as a sample containing blood lymphocytes. In some instances, binding molecules, e.g., TCRs, may be amplified out of the sample, e.g., T cells contained in the sample. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen. In some aspects, the TCRs are subjected to directed evolution, such as by mutagenesis, e.g., of the α or β chain. In some aspects, particular residues within CDRs of the TCR are altered. In some embodiments, selected TCRs may be modified by affinity maturation. In some aspects, a selected TCR may be used as a parent scaffold TCR against the antigen.

In some embodiments, the subject is a human, such as a human with a disorder characterized by MAGEC2 expression. In some embodiments, the subject is a rodent, such as a mouse. In some such embodiments, the mouse is a transgenic mouse, such as a mouse expressing human MHC (i.e., HLA) molecules, such as HLA-A2 (e.g., Nicholson et al. (2012) Adv. Hematol. 2012:404081).

In some embodiments, the subject is a transgenic mouse expressing human TCRs or is an antigen-negative mouse (e.g., Li et al. (2010) Nat Med. 161029-1034; Obenaus et al. (2015) Nat. Biotechnol. 33:402-407). In some embodiments, the subject is a transgenic mouse expressing human HLA molecules and human TCRs.

In some embodiments, such as where the subject is a transgenic HLA mouse, the identified TCRs are modified, e.g., to be chimeric or humanized. In some aspects, the TCR scaffold is modified, such as analogous to known antibody humanizing methods.

In some embodiments, such a scaffold molecule is used to generate a library of TCRs.

For example, in some embodiments, the library includes TCRs or antigen-binding portions thereof that have been modified or engineered compared to the parent or scaffold TCR molecule. In some embodiments, directed evolution methods may be used to generate TCRs with altered properties, such as with higher affinity for a specific MHC-peptide complex. In some embodiments, display approaches involve engineering, or modifying, a known, parent or reference TCR. For example, in some cases, a wild-type TCR may be used as a template for producing mutagenized TCRs in which in one or more residues of the CDRs are mutated, and mutants with an desired altered property, such as higher affinity for a desired target antigen, are selected. In some embodiments, directed evolution is achieved by display methods including, but not limited to, yeast display (Holler et al. (2003) Nat. Immunol. 4:55-62; Holler et al. (2000) Proc. Natl. Acad. Sci. U.S.A. 97:5387-5392), phage display (Li et al. (2005) Nat. Biotechnol. 23:349-354), or T cell display (Chervin et al. (2008) J. Immunol. Methods 339:175-184).

In some embodiments, the libraries may be soluble. In some embodiments, the libraries are display libraries in which the TCR is displayed on the surface of a phage or cell, or attached to a particle or molecule, such as a cell, ribosome or nucleic acid, e.g., RNA or DNA. Typically, the TCR libraries, including normal and disease TCR libraries or diversified libraries, may be generated in any form, including as a heterodimer or as a single chain form. In some embodiments, one or more members of the TCR may be a two-chain heterodimer. In some embodiments, pairing of the Vα and Vβ chains may be promoted by introduction of a disulfide bond. In some embodiments, members of the TCR library may be a TCR single chain (scTv or ScTCR), which, in some cases, may include a Vα and Vβ chain separated by a linker. Further, in some cases, upon screening and selection of a TCR from the library, the selected member may be generated in any form, such as a full-length TCR heterodimer or single-chain form or as antigen-binding fragments thereof.

Other methods of identifying molecules that bind to a peptide in the context of an MHC molecule are also described in U.S. Pat. Appl. No. 2020/0182884.

More generally, the present invention encompasses assays for screening agents, such as test proteins, that bind to, or modulate the activity of, MAGEC2 or an antigen thereof. Such agents include, without limitation, antibodies, proteins, fusion proteins, small molecules, and nucleic acids. In some embodiments, a method for identifying an agent which modulates an immune response entails determining the ability of the candidate agent to modulate MAGEC2 activity and further modulate an immune response of interest, such as modulated cytotoxic T cell activation and/or activity, sensitivity of cancer cells to immune checkpoint therapy, and the like.

In some embodiments, an assay is a cell-free or cell-based assay, comprising contacting a target, with a test agent, and determining the ability of the test agent to modulate (e.g., upregulate or downregulate) the amount and/or activity of the target, such as by measuring direct or indirect parameters as described below.

In some embodiments, an assay is a cell-based assay, such as one comprising contacting (a) a cell of interest with a test agent and determining the ability of the test agent to modulate the amount and/or activity of the target, such as binding characteristics. Determining the ability of the polypeptides to bind to, or interact with, each other may be accomplished, e.g., by measuring direct binding or by measuring a parameter of immune cell activation or function.

In another embodiment, an assay is a cell-based assay, comprising contacting a cell such as a cancer cell with immune cells (e.g., cytotoxic T cells) and a test agent, and determining the ability of the test agent to modulate the amount and/or activity of the target, and/or modulated immune responses, such as by measuring direct or indirect parameters as described below.

The methods described above and herein may also be adapted to test one or more agents that are already known to modulate the amount and/or activity of one or more biomarkers described herein to confirm modulation of the one or more biomarkers and/or to confirm the effects of the agents on readouts of a desired phenotype, such as modulated immune responses, sensitivity to immune checkpoint blockade, and the like.

In a direct binding assay, biomarker protein (or their respective target polypeptides or molecules) may be coupled with a radioisotope or enzymatic label such that binding may be determined by detecting the labeled protein or molecule in a complex. For example, the targets may be labeled with ¹²⁵I, ³⁵S, ¹⁴C, or ³H, either directly or indirectly, and the radioisotope detected by direct counting of radioemmission or by scintillation counting. Alternatively, the targets may be enzymatically labeled with, for example, horseradish peroxidase, alkaline phosphatase, or luciferase, and the enzymatic label detected by determination of conversion of an appropriate substrate to product. Determining the interaction between target and substrate may also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target may be accomplished in any vessel suitable for containing the reactants. Non-limiting examples of such vessels include microtiter plates, test tubes, and micro-centrifuge tubes. Immobilized forms of the antibodies encompassed by the present invention may also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

For example, in a direct binding assay, the polypeptides may be coupled with a radioisotope or enzymatic label such that polypeptide interactions and/or activity, such as binding events, may be determined by detecting the labeled protein in a complex. For example, the polypeptides may be labeled with ¹²⁵I, ³⁵S, ¹⁴C, or ³H, either directly or indirectly, and the radioisotope detected by direct counting of radioemmission or by scintillation counting. Alternatively, the polypeptides may be enzymatically labeled with, for example, horseradish peroxidase, alkaline phosphatase, or luciferase, and the enzymatic label detected by determination of conversion of an appropriate substrate to product.

It is also within the scope of the present invention to determine the ability of an agent to modulate a parameter of interest without the labeling of any of the interactants. For example, a microphysiometer may be used to detect interaction between polypeptides without the labeling of polypeptides to be monitored (McConnell et al. (1992) Science 257:1906-1912). As used herein, a “microphysiometer” (e.g., Cytosensor®) is an analytical instrument that measures the rate at which a cell acidifies its environment using a light-addressable potentiometric sensor (LAPS). Changes in this acidification rate may be used as an indicator of the interaction between compound and receptor.

In some embodiments, determining the ability of a test agent (e.g. antibodies, fusion proteins, peptides, or small molecules) to modulate the interaction between a given set of polypeptides may be accomplished by determining the activity of one or more members of the set of polypeptides. For example, the activity of a protein and/or one or more binding partners may be determined by detecting induction of a cellular second messenger (e.g., intracellular signaling), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the protein and/or the one or more binding partners. Determining the ability of the test agent to bind to or interact with said polypeptide may be accomplished, for example, by measuring the ability of a compound to modulate immune cell costimulation or inhibition in a proliferation assay, or by interfering with the ability of said polypeptide to bind to antibodies that recognize a portion thereof.

Agents that modulate target amount and/or activity, such as interactions with one or more binding partners, may be identified by their ability to inhibit immune cell proliferation, and/or effector function, or to induce anergy, clonal deletion, and/or exhaustion when added to an in vitro assay. For example, cells may be cultured in the presence of an agent that stimulates signal transduction via an activating receptor. A number of recognized readouts of cell activation may be employed to measure, cell proliferation or effector function (e.g., antibody production, cytokine production, phagocytosis) in the presence of the agent. The ability of a test agent to block this activation may be readily determined by measuring the ability of the agent to effect a decrease in proliferation or effector function being measured, using techniques known in the art.

For example, agents encompassed by the present invention may be tested for the ability to inhibit or enhance costimulation in a T cell assay, as described in Freeman et al. (2000) J Exp. Med. 192:1027 and Latchman et al. (2001) Nat. Immunol. 2:261. CD4+ T cells may be isolated from human PBMCs and stimulated with activating anti-CD3 antibody. Proliferation of T cells may be measured by ³H thymidine incorporation. An assay may be performed with or without CD28 costimulation in the assay. Similar assays may be performed with Jurkat T cells and PHA-blasts from PBMCs.

Alternatively, agents encompassed by the present invention may be tested for the ability to modulate cellular production of cytokines which are produced by or whose production is enhanced or inhibited in immune cells in response to modulation of the one or more biomarkers. Indicative cytokines released by immune cells of interest may be identified by ELISA or by the ability of an antibody which blocks the cytokine to inhibit immune cell proliferation or proliferation of other cell types that is induced by the cytokine, such as those described in the Examples section. An in vitro immune cell costimulation assay may also be used in a method for identifying cytokines which may be modulated by modulation of the one or more biomarkers. For example, if a particular activity induced upon costimulation, e.g., immune cell proliferation, cannot be inhibited by addition of blocking antibodies to known cytokines, the activity may result from the action of an unknown cytokine. Following costimulation, this cytokine may be purified from the media by conventional methods and its activity measured by its ability to induce immune cell proliferation. To identify cytokines which may play a role the induction of tolerance, an in vitro T cell costimulation assay as described above may be used. In this case, T cells would be given the primary activation signal and contacted with a selected cytokine, but would not be given the costimulatory signal. After washing and resting the immune cells, the cells would be rechallenged with both a primary activation signal and a costimulatory signal. If the immune cells do not respond (e.g., proliferate or produce cytokines) they have become tolerized and the cytokine has not prevented the induction of tolerance. However, if the immune cells respond, induction of tolerance has been prevented by the cytokine. Those cytokines which are capable of preventing the induction of tolerance may be targeted for blockage in vivo in conjunction with reagents which block B lymphocyte antigens as a more efficient means to induce tolerance in transplant recipients or subjects with autoimmune diseases.

In some embodiments, an assay encompassed by the present invention is a cell-free assay for screening for agents that modulate the interaction between a biomarker and/or one or more binding partners, comprising contacting a polypeptide and one or more natural binding partners, or biologically active portion thereof, with a test agent and determining the ability of the test compound to modulate the interaction between the polypeptide and one or more natural binding partners, or biologically active portion thereof. Binding of the test compound may be determined either directly or indirectly as described above. In one embodiment, the assay includes contacting the polypeptide, or biologically active portion thereof, with its binding partner to form an assay mixture, contacting the assay mixture with a test compound, and determining the ability of the test agent to interact with the polypeptide in the assay mixture, wherein determining the ability of the test agent to interact with the polypeptide comprises determining the ability of the test agent to preferentially bind to the polypeptide or biologically active portion thereof, as compared to the binding partner.

In some embodiments, whether for cell-based or cell-free assays, a test agent may further be assayed to determine whether it affects binding and/or activity of the interaction between the polypeptide and the one or more binding partners, with other binding partners. Other useful binding analysis methods include the use of real-time Biomolecular Interaction Analysis (BIA) (Sjolander and Urbaniczky (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., Biacore®). Changes in the optical phenomenon of surface plasmon resonance (SPR) may be used as an indication of real-time reactions between biological polypeptides. Polypeptides of interest may be immobilized on a Biacore® chip and multiple agents (blocking antibodies, fusion proteins, peptides, or small molecules) may be tested for binding to the polypeptide of interest. An example of using the BIA technology is described by Fitz et al. (1997) Oncogene 15:613.

The cell-free assays encompassed by the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), orN-dodecyl=N,N-dimethyl-3-ammonio-1-propane sulfonate.

In one or more embodiments of the above described assay methods, it may be desirable to immobilize either polypeptides to facilitate separation of complexed from uncomplexed forms of one or both of the proteins, as well as to accommodate automation of the assay. Binding of a test compound to a polypeptide, may be accomplished in any vessel suitable for containing the reactants. Examples of such vessels include microtiter plates, test tubes, and micro-centrifuge tubes. In one embodiment, a fusion protein may be provided which adds a domain that allows one or both of the proteins to be bound to a matrix. For example, glutathione-S-transferase-based polypeptide fusion proteins, or glutathione-S-transferase/target fusion proteins, may be adsorbed onto glutathione sepharose beads (Sigma Chemical, St. Louis, MO) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes may be dissociated from the matrix, and the level of polypeptide binding or activity determined using standard techniques.

The present invention further pertains to novel agents identified by the above-described screening assays. Accordingly, it is within the scope of the present invention to further use an agent identified as described herein in an appropriate model system. For example, an agent identified as described herein may be used in a model system to determine the efficacy, toxicity, or side effects of treatment with such an agent. Alternatively, an agent identified as described herein may be used in a model system to determine the mechanism of action of such an agent. Furthermore, the present invention pertains to uses of novel agents identified by the above-described screening assays for treatments as described herein.

d. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect encompassed by the present invention encompasses diagnostic assays for determining (e.g., detecting) the presence, absence, amount, and/or activity level of MAGEC2 or reactivity to MAGEC2 in the context of a biological sample (e.g., blood, serum, cells, or tissue) to thereby determine whether an individual afflicted with a disorder characterized by MAGEC2 expression is likely to respond to therapy, whether in an original state or as a recurrence. Such assays may be used for prognostic or predictive purpose to thereby prophylactically treat an individual prior to the onset or after recurrence of a disorder characterized by MAGEC2 expression.

The diagnostic methods described herein may furthermore be utilized to identify subjects having or at risk of developing a disorder associated with expression or lack thereof of MAGEC2. As used herein, the term “aberrant” includes an upregulation or downregulation of MAGEC2 from normal levels. Aberrant expression or activity includes increased or decreased expression or activity, as well as expression or activity which does not follow the normal developmental pattern of expression or the subcellular pattern of expression. For example, aberrant levels is intended to include the cases in which a mutation in the biomarker gene or regulatory sequence, or amplification of the chromosomal gene, thereof causes upregulation or downregulation of the biomarker of interest. As used herein, the term “unwanted” includes an unwanted phenomenon involved in a biological response, such as immune cell activity.

The assays described herein, such as the preceding diagnostic assays or the following assays, may be utilized to identify a subject having or at risk of developing a disorder associated with MAGEC2 misregulation. Thus, the present invention provides a method for identifying a disorder associated with aberrant or unwanted MAGEC2 regulation in which a test sample is obtained from a subject and MAGEC2 expression is detected, wherein the presence of MAGEC2 expression is diagnostic for a subject having or at risk of developing the disorder associated with aberrant or unwanted MAGEC2 expression. As used herein, a “test sample” refers to a biological sample obtained from a subject of interest. For example, a test sample may be a biological fluid (e.g., cerebrospinal fluid or serum), cell sample, or tissue, such as a histopathological slide of the tumor microenvironment, peritumoral area, and/or intratumoral area.

Furthermore, the prognostic assays described herein may be used to determine whether a subject may be administered an agent described herein to treat such a disorder associated with aberrant or unwanted MAGEC2 expression. For example, such methods may be used to determine whether a subject may be effectively treated with one or a combination of agents. Thus, the present invention provides methods for determining whether a subject may be effectively treated with one or more agents described herein for treating a disorder associated with aberrant or unwanted MAGEC2 expression.

The methods described herein may be performed, for example, by utilizing pre-packaged diagnostic kits comprising at least one antibody reagent described herein, which may be conveniently used, e.g., in clinical settings to diagnose patients exhibiting symptoms or family history of a disease or illness involving the biomarker of interest.

Furthermore, any cell type or tissue in which the biomarker of interest is expressed may be utilized in the prognostic assays described herein.

e. Monitoring of Effects During Clinical Trials

Monitoring the influence of a disorder characterized by MAGEC2 expression therapy (e.g., compounds, drugs, vaccines, cell therapies, and the like) on immune responses, such as T cell reactivity (e.g., the presence of binding and/or T cell activation and/or effector function), may be applied not only in basic candidate MAGEC2 antigen binding molecule screening, but also in clinical trials. For example, the effectiveness of immunogenic peptides, pMHCs, engineered cells, binding proteins, and related compostions described herein to increase an immune response (e.g., T cell immune response) against cells of interest, such as hyperproliferative cells, expressing MAGEC2, may be monitored in clinical trials of subjects afflicted with a disorder characterized by MAGEC2 expression. In such clinical trials, the presence of binding and/or T cell activation and/or effector function (e.g., T cell proliferation, killing, and/or cytokine release), may be used as a “read out” or marker of the phenotype of a particular cell, tissue, or system. Similarly, the effectiveness of an adaptive T cell therapy with T cells engineered to express a binding protein (e.g., a TCR, an antigen-binding fragment of a TCR, a CAR, or a fusion protein comprising a TCR and an effector domain) as described herein to increase immune response to cells of interest, such as hyperproliferative cells, that are expressing MAGEC2, may be monitored in clinical trials of subjects having a disorder characterized by MAGEC2 expression. In such clinical trials, the presence of binding and/or T cell activation and/or effector function (e.g., T cell proliferation, killing, or cytokine release), may be used as a “read out” or marker of the phenotype of a particular cell, tissue, or system.

In some embodiments, the present invention provides a method for monitoring the effectiveness of treatment of a therapy (e.g., compounds, drugs, vaccines, cell therapies, and the like) including the steps of a) determining the absence, presence, or level of reactivity between a sample obtained from the subject and one or more binding proteins or related composition, in a first sample obtained from the subject prior to providing at least a portion of the therapy for the disorder characterized by MAGEC2 expression to the subject, and b) determining the absence, presence, or level of reactivity between the one or more binding proteins or related composition, and a sample obtained from the subject present in a second sample obtained from the subject following provision of the portion of the therapy, wherein the presence or a higher level of reactivity in the first sample, relative to the second sample, is an indication that the therapy is efficacious for treating the disorder characterized by MAGEC2 expression in the subject and wherein the absence or a lower level of reactivity in the first sample, relative to the second sample, is an indication that the therapy is not efficacious for treating the disorder characterized by MAGEC2 expression in the subject.

In some embodiments, the present invention provides a method for monitoring the effectiveness of treatment of a subject with an agent (e.g., antibodies, an agonist, antagonist, peptidomimetic, polypeptide, peptide, nucleic acid, small molecule, or other drug candidate identified by the screening assays described herein) including the steps of (i) obtaining a pre-administration sample from a subject prior to administration of the agent; (ii) detecting MAGEC2 expression in the preadministration sample; (iii) obtaining one or more post-administration samples from the subject; (iv) detecting MAGEC2 expression in the post-administration samples; (v) comparing the MAGEC2 expression in the pre-administration sample with the MAGEC2 expression in the post-administration sample; and (vi) altering the administration of the agent to the subject accordingly. Biomarker polypeptide analysis, such as by immunohistochemistry (IHC), may also be used to select patients who will receive therapy, such as immunotherapy.

In addition, the prognostic methods described herein may be used to determine whether a subject may be administered a therapeutic agent to treat a disorder associated with MAGEC2 expression.

f. Clinical Efficacy

Clinical efficacy may be measured by any method known in the art. For example, the response to a therapy relates to any response of the disorder associated with MAGEC2 expression, e.g., a tumor, to the therapy, preferably to a change in the number of cancer cells, tumor mass, and/or tumor volume, such as after initiation of neoadjuvant or adjuvant chemotherapy. Tumor response may be assessed in a neoadjuvant or adjuvant situation where the size of a tumor after systemic intervention may be compared to the initial size and dimensions as measured by CT, PET, mammogram, ultrasound or palpation and the cellularity of a tumor may be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion such as percentage change in tumor volume or cellularity or by using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy (e.g., after a few hours, days, weeks or preferably after a few months). A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular modulator of biomarkers listed in Table 1 therapeutic regimen is at least 25%, 30%, 35%, 40%, 45%, 50%, 55%, 60%, 65%, 70%, 75%, 80%, 85%, or more.

Additional criteria for evaluating the response to cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence, or metastasis). In addition, criteria for efficacy of treatment may be expanded to include response to chemotherapy, probability of survival, probability of metastasis within a given time period, and probability of tumor recurrence.

For example, in order to determine appropriate threshold values, a particular agent of interest may be administered to a population of subjects and the outcome may be correlated to biomarker measurements that were determined prior to administration of any therapy. The outcome measurement may be pathologic response to therapy given in the neoadjuvant setting. Alternatively, outcome measures, such as overall survival and disease-free survival may be monitored over a period of time for subjects following therapy for whom MAGEC2 expression values are known. In certain embodiments, the same doses of the agent are administered to each subject. The period of time for which subjects are monitored may vary. For example, subjects may be monitored for at least 1, 2, 3, 4, 5, 6, 7, 8, 9, 10, 11, 12, 14, 16, 18, 20, 25, 30, 35, 40, 45, 50, 55, 60 months, or longer. MAGEC2 measurement threshold values that correlate to outcome of a therapy may be determined using well-known methods, such as those described in the Examples section.

### X. Cell Therapy

In another aspect encompassed by the present invention, the methods include adoptive cell therapy, whereby genetically engineered cells expressing the provided molecules targeting an MHC-restricted epitope (e.g., cells expressing a binding protein (e.g., a TCR or CAR) or antigen-binding fragment thereof) are administered to subjects. Such administration may promote activation of immune cells (e.g., T cell activation) in an antigen-targeted manner, such that the cells of interest, such as hyperproliferative cells, that express MAGEC2 are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated (e.g., via adoptive cell therapy, such as by adoptive T cell therapy). In some embodiments, the cells or compositions are administered to the subject, such as a subject having or at risk for the disease or condition. In some embodiments, the methods thereby treat, e.g., ameliorate one or more symptom of the disease or condition.

Methods for administration of cells for adoptive cell therapy are known and may be used in connection with the provided methods and compositions (e.g., U.S. Pat. Publ. No. 2003/0170238, U.S. Pat. No. 4,690,915, Rosenberg (2011) Nat. Rev. Clin. Oncol. 8:577-585, Themeli et al. (2013) Nat. Biotechnol. 31:928-933, Tsukahara et al. (2013) Biochem. Biophys. Res. Commun. 438:84-89, and Davila et al. (2013) PLoS ONE 8:e61338).

In some embodiments, cell therapy (e.g., adoptive cell therapy, such as adoptive T cell therapy) may be carried out by autologous transfer, in which the cells are isolated and/or otherwise prepared from the subject who is to receive the cell therapy, or from a sample derived from such a subject. Thus, in some embodiments, the cells are derived from a subject (e.g., patient) in need of a treatment and the cells, following isolation and processing are administered to the same subject.

In some embodiments, the cell therapy (e.g., adoptive cell therapy, such as adoptive T cell therapy) may be carried out by allogeneic transfer, in which the cells are isolated and/or otherwise prepared from a subject other than a subject who is to receive or who ultimately receives the cell therapy (e.g., a first subject). In such embodiments, the cells then are administered to a different subject (e.g., a second subject) of the same species. In some embodiments, the first and second subjects are genetically identical (syngeneic). In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The binding molecules, such as TCRs, antigen-binding fragments of TCRs (e.g., scTCRs) and chimeric receptors (e.g., CARs) containing the TCR, and cells expressing the same, may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

In some embodiments, cells may be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective. For example, in some embodiments, cells, or individual populations of sub-types of cells, may be administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or may signal the time for tumor harvesting. Any such metrics are variables that may be adjusted according to well-known parameters in order to determine the effect of the variable on a response to anti-cancer immunotherapy. In addition, the transplanted cells may be co-transplanted with other agents, such as cytokines, extracellular matrices, cell culture supports, and the like.

Cells may also be administered before, concurrently with, or after, other anti-cancer agents.

Two or more cell types may be combined and administered, such as cell-based therapy and adoptive cell transfer of stem cells, cancer vaccines and cell-based therapy, and the like. For example, adoptive cell-based immunotherapies may be combined with the cell-based therapies encompassed by the present invention. In some embodiments, the cell-based agents may be used alone or in combination with additional cell-based agents, such as immunotherapies like adoptive T cell therapy (ACT). For example, T cells genetically engineered to recognize CD19 used to treat follicular B cell lymphoma. Immune cells for ACT may be dendritic cells, T cells such as CD8⁺ T cells and CD4⁺ T cells, natural killer (NK) cells, NK T cells, cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies may be further modified to express one or more gene products to further modulate immune responses, such as expressing cytokines like GM-CSF, and/or to express tumor-associated antigen (TAA) antigens, such as Mage-1, gp-100, and the like. The ratio of an agent encompassed by the present invention, such as cancer cells, to another agent encompassed by the present invention or other composition may be 1:1 relative to each other (e.g., equal amounts of 2 agents, 3 agents, 4 agents, etc.), but may modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

In some embodiments, for example, where the subject is a human, the dose includes fewer than about 1×10⁸ total binding protein (e.g., TCR- or CAR-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or related compositions described herein, such as nucleic acids, host cells, pharmaceutical formulations, and the like, may be administered as part of a combination treatment, such as simultaneously with or sequentially with, in any order, another therapeutic intervention, such as another antibody or engineered cell or receptor or agent, such as a cytotoxic or therapeutic agent.

In some embodiments, the cells or related composition may be co-administered with one or more additional therapeutic agents or in connection with another therapeutic intervention, either simultaneously or sequentially in any order. In some contexts, the cells or related composition are co-administered with another therapy sufficiently close in time such that the cell populations enhance the effect of one or more additional therapeutic agents, or vice versa. In some embodiments, the cells or related composition are administered prior to the one or more additional therapeutic agents. In some embodiments, the cells or related composition are administered after to the one or more additional therapeutic agents.

In some embodiments, the biological activity of the cells or related composition is measured by any of a number of known methods once the cells or related composition are administered to a subject (e.g., a human). Parameters to assess include specific binding of an engineered or natural T cell or other immune cell to antigen, in vivo, e.g., by imaging, or in vitro/ex vivo, e.g., by ELISA or flow cytometry. In some embodiments, the ability of the cells to destroy target cells (cytotoxicity) may be measured using any suitable assay or method known in the art (e.g., Kochenderfer et al. (2009) J. Immunother. 32: 689-702 and Herman et al. (2004) J. Immunol. Meth. 285:25-40). In some embodiments, the biological activity of the cells also may be measured by assaying expression and/or secretion of certain cytokines, such as CD107a, IFNγ, IL-2, and TNF alpha. In some embodiments, the biological activity is measured by assessing clinical outcome, such as reduction in viral burden or load.

In some embodiments, cells are modified in any number of ways, such that their therapeutic or prophylactic efficacy is increased. For example, the binding protein (e.g., engineered TCR, CAR, or antigen-binding fragment thereof) expressed by the population may be conjugated either directly or indirectly through a linker to a targeting moiety. The practice of conjugating compounds to targeting moieties is well-known in the art (e.g., Wadwa et al. (1995) J Drug Targeting 3:111 and U.S. Pat. No. 5,087,616).

Immune cells, such as cytotoxic lymphocytes, may be obtained from any suitable source such as peripheral blood, spleen, and lymph nodes. The immune cells may be used as crude preparations or as partially purified or substantially purified preparations, which may be obtained by standard techniques, including, but not limited to, methods involving immunomagnetic or flow cytometry techniques using antibodies.

In certain aspects, the MAGEC2 immunogenic peptides described herein, or a nucleic acid encoding such MAGEC2 immunogenic peptides, may be used in compositions and methods for providing MAGEC2-primed, antigen-presenting cells, and/or MAGEC2-specific lymphocytes generated with these antigen-presenting cells. In some embodiments, such antigen-presenting cells and/or lymphocytes are used in the treatment and/or prevention of a disorder associated with MAGEC2 expression.

In some aspects, provided herein are methods for making MAGEC2-primed, antigen-presenting cells by contacting antigen-presenting cells with a MAGEC2 immunogenic peptide described herein, or nucleic acids encoding the at least one MAGEC2 immunogenic peptide, alone or in combination with an adjuvant, in vitro under a condition sufficient for the at least one MAGEC2 immunogenic polypeptide to be presented by the antigen-presenting cells.

In some embodiments, MAGEC2 immunogenic polypeptide, or nucleic acid encoding the MAGEC2 immunogenic polypeptide, alone or in combination with an adjuvant, may be contacted with a homogenous, substantially homogenous, or heterogeneous composition comprising antigen-presenting cells. For example, the composition may include but is not limited to whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells, and natural killer T cells. If, optionally, precursors of antigen-presenting cells are used, the precursors may be cultured under suitable culture conditions sufficient to differentiate the precursors into antigen-presenting cells. In some embodiments, the antigen-presenting cells (or precursors thereof) are selected from monocytes, macrophages, cells of myeloid lineage, B cells, dendritic cells, or Langerhans cells.

The amount of the MAGEC2 immunogenic polypeptide, or nucleic acid encoding the MAGEC2 immunogenic polypeptide, alone or in combination with an adjuvant, to be placed in contact with antigen-presenting cells may be determined by one of ordinary skill in the art by routine experimentation. Generally, antigen-presenting cells are contacted with the MAGEC2 immunogenic polypeptide, or nucleic acid encoding the MAGEC2 immunogenic polypeptide, alone or in combination with an adjuvant, for a period of time sufficient for cells to present the processed forms of the antigens for the modulation of T cells. In one embodiment, antigen-presenting cells are incubated in the presence of the MAGEC2 immunogenic polypeptide, or nucleic acid encoding the MAGEC2 immunogenic polypeptide, alone or in combination with an adjuvant, for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, and about 40 minutes to about 1 hour. The time and amount of the MAGEC2 immunogenic polypeptide, or nucleic acid encoding the MAGEC2 immunogenic polypeptide, alone or in combination with an adjuvant, necessary for the antigen presenting cells to process and present the antigens may be determined, for example using pulse-chase methods wherein contact is followed by a washout period and exposure to a read-out system e.g., antigen reactive T cells.

In certain embodiments, any appropriate method for delivery of antigens to the endogenous processing pathway of the antigen-presenting cells may be used. Such methods include but are not limited to, methods involving pH-sensitive liposomes, coupling of antigens to adjuvants, apoptotic cell delivery, pulsing cells onto dendritic cells, delivering recombinant chimeric virus-like particles (VLPs) comprising antigen to the MHC class I processing pathway of a dendritic cell line.

In one embodiment, solubilized MAGEC2 immunogenic polypeptide is incubated with antigen-presenting cells. In some embodiments, the MAGEC2 immunogenic polypeptide may be coupled to a cytolysin to enhance the transfer of the antigens into the cytosol of an antigen-presenting cell for delivery to the MHC class I pathway. Exemplary cytolysins include saponin compounds such as saponin-containing Immune Stimulating Complexes (ISCOM5), pore-forming toxins (e.g., an alpha-toxin), and natural cytolysins of gram-positive bacteria such as listeriolysin O (LLO), streptolysin O (SLO), and perfringolysin O (PFO).

In some embodiments, antigen-presenting cells, such as dendritic cells and macrophage, may be isolated according to methods known in the art and transfected with polynucleotides by methods known in the art for introducing a nucleic acid encoding the MAGEC2 immunogenic polypeptide into the antigen-presenting cell. Transfection reagents and methods are known in the art and commercially available. For example, RNA encoding MAGEC2 immunogenic polypeptide may be provided in a suitable medium and combined with a lipid (e.g., a cationic lipid) prior to contact with antigen-presenting cells. Non-limiting examples of such lipids include LIPOFECTIN™ and LIPOFECTAMINE™. The resulting polynucleotide-lipid complex may then be contacted with antigen-presenting cells. Alternatively, the polynucleotide may be introduced into antigen-presenting cells using techniques such as electroporation or calcium phosphate transfection. The polynucleotide-loaded antigen-presenting cells may then be used to stimulate T lymphocyte (e.g., cytotoxic T lymphocyte) proliferation in vitro, ex vivo, or in vivo. In one embodiment, the ex vivo expanded T lymphocyte is administered to a subject in a method of adoptive immunotherapy.

In certain aspects, provided herein is a composition comprising antigen-presenting cells that have been contacted in vitro with a MAGEC2 immunogenic polypeptide, or a nucleic acid encoding a MAGEC2 immunogenic polypeptide, alone or in combination with an adjuvant under a condition sufficient for a MAGEC2 immunogenic epitope to be presented by the antigen-presenting cells.

In some aspects, provided herein is a method for preparing lymphocytes specific for a MAGEC2 protein. The method comprises contacting lymphocytes with the antigen-presenting cells described above under conditions sufficient to produce a MAGEC2 protein-specific lymphocyte capable of eliciting an immune response against a cell that is infected by the MAGEC2 virus. Thus, the antigen-presenting cells also may be used to provide lymphocytes, including T lymphocytes and B lymphocytes, for eliciting an immune response against cell that is infected by the MAGEC2 virus.

In some embodiments, a preparation of T lymphocytes is contacted with the antigen-presenting cells described above for a period of time, (e.g., at least about 24 hours) to priming the T lymphocytes to a MAGEC2 immunogenic epitope presented by the antigen-presenting cells.

In some embodiments, a population of antigen-presenting cells may be co-cultured with a heterogeneous population of peripheral blood T lymphocytes together with a MAGEC2 immunogenic polypeptide, or a nucleic acid encoding a MAGEC2 immunogenic polypeptide, alone or in combination with an adjuvant. The cells may be co-cultured for a period of time and under conditions sufficient for MAGEC2 epitopes included in the MAGEC2 polypeptides to be presented by the antigen-presenting cells and the antigen-presenting cells to prime a population of T lymphocytes to respond to cells is infected by the MAGEC2 virus. In certain embodiments, provided herein are T lymphocytes and B lymphocytes that are primed to respond to cells that is infected by the MAGEC2 virus.

T lymphocytes may be obtained from any suitable source such as peripheral blood, spleen, and lymph nodes. The T lymphocytes may be used as crude preparations or as partially purified or substantially purified preparations, which may be obtained by standard techniques including, but not limited to, methods involving immunomagnetic or flow cytometry techniques using antibodies.

In certain aspects, provided herein is a composition (e.g., a pharmaceutical composition) comprising the antigen-presenting cells or the lymphocytes described above, and a pharmaceutically acceptable carrier and/or diluent. In some embodiments, the composition further comprises an adjuvant as described above.

In certain aspects and as further described above, provided herein is a method for eliciting an immune response to the cell is infected by the MAGEC2 virus, the method comprising administering to the subject the antigen-presenting cells or the lymphocytes described above in effective amounts sufficient to elicit the immune response. In some embodiments, provided herein is a method for treatment or prophylaxis of a disorder characterized by MAGEC2 expression, the method comprising administering to the subject an effective amount of the antigen-presenting cells or the lymphocytes described above. In one embodiment, the antigen-presenting cells or the lymphocytes are administered systemically, preferably by injection. Alternately, one may administer locally rather than systemically, for example, via injection directly into tissue, preferably in a depot or sustained release formulation.

In certain embodiments, the antigen-primed antigen-presenting cells described herein and the antigen-specific T lymphocytes generated with these antigen-presenting cells may be used as active compounds in immunomodulating compositions for prophylactic or therapeutic treatment of a disorder characterized by MAGEC2 expression. In some embodiments, the MAGEC2-primed antigen-presenting cells described herein may be used for generating CD8⁺ T lymphocytes, CD4⁺ T lymphocytes, and/or B lymphocytes for adoptive transfer to the subject. Thus, for example, MAGEC2-specific lymphocyte may be adoptively transferred for therapeutic purposes in subjects afflicted with a disorder characterized by MAGEC2 expression.

In certain embodiments, the antigen-presenting cells and/or lymphocytes described herein may be administered to a subject, either by themselves or in combination, for eliciting an immune response, particularly for eliciting an immune response to cells expressing MAGEC2. In some embodiments, the antigen-presenting cells and/or lymphocytes may be derived from the subject (i.e., autologous cells) or from a different subject that is MHC matched or mismatched with the subject (e.g., allogeneic).

Single or multiple administrations of the antigen-presenting cells and lymphocytes may be carried out with cell numbers and treatment being selected by the care provider (e.g., physician). In some embodiments, the antigen-presenting cells and/or lymphocytes are administered in a pharmaceutically acceptable carrier. Suitable carriers may be growth medium in which the cells were grown, or any suitable buffering medium such as phosphate buffered saline. The cells may be administered alone or as an adjunct therapy in conjunction with other therapeutics.

In another aspect encompassed by the present invention, provided herein is a method for eliciting an immune response to a cell that expresses MAGEC2, the method comprising administering to the subject cells described herein expressing a binding protein (e.g., engineered TCR, CAR, or antigen-binding fragment thereof) in effective amounts sufficient to elicit the immune response. In some embodiments, provided herein is a method for treatment or prophylaxis of a disorder characterized by MAGEC2 expression, the method comprising administering to the subject an effective amount of the cells described herein expressing a binding protein (e.g., engineered TCR, CAR, or antigen-binding fragment thereof). In one embodiment, the cells are administered systemically, such as by injection. Alternately, one may administer locally rather than systemically, for example, via injection directly into tissue, such as in a depot or sustained release formulation.

In some embodiments, the cells described herein expressing a binding protein (e.g., engineered TCR, CAR, or antigen-binding fragment thereof) may be used as active compounds in immunomodulating compositions for prophylactic or therapeutic treatment of a disorder characterized by MAGEC2 expression. In some embodiments, MAGEC2-primed antigen-presenting cells may be used for generating lymphocytes (e.g., CD8⁺ T lymphocytes, CD4⁺ T lymphocytes, and/or B lymphocytes), for further use in adoptive transfer to the subject with the cells described herein expressing a binding protein (e.g., engineered TCR, CAR, or antigen-binding fragment thereof).

In some embodiments, the cells described herein expressing a binding protein (e.g., engineered TCR, CAR, or antigen-binding fragment thereof), either alone or in combination with the lymphocytes, may be administered to a subject for eliciting an immune response, particularly for eliciting an immune response to cells are expressing MAGEC2.

As described above, single or multiple administrations of the cells described herein expressing a binding protein (e.g., engineered TCR, CAR, or antigen-binding fragment thereof) cells, either alone or in combination with the lymphocytes, may be carried out with cell numbers and treatment being selected by the care provider (e.g., physician). Similarly, the cells, either alone or in combination with lymphocytes, may be administered in a pharmaceutically acceptable carrier. Suitable carriers may be growth medium in which the cells were grown, or any suitable buffering medium such as phosphate buffered saline. Cells may be administered alone or as an adjunct therapy in conjunction with other therapeutics.

### XI. Kits and Devices

The present invention also encompasses kits and devices. For example, the kit or device may comprise binding proteins, nucleic acids or vectors comprising sequences encoding binding proteins, host cells comprising nucleic acids or vectors and/or expressing the binding proteins as described herein, stable MHC-peptide complexes, adjuvants, detection reagents, and combinations thereof, packaged in a suitable container and may further comprise instructions for using such reagents. The kit may also contain other components, such as administration tools packaged in a separate container. The kit may be promoted, distributed, or sold as a unit for performing the methods encompassed by the present invention.

The disclosure is further illustrated by the following examples, which should not be construed as limiting.

### EXAMPLES

### Example 1: Materials and Methods for Example 2

a. Immunogenic Epitope Identification

### (i) Discovery and Design of TCR Pool

Paired TCR alpha and TCR beta sequences were obtained by single cell sequencing (10X Genomics) of TIL therapy products following manufacturer's instructions for the Chromium™ Single Cell V(D)J Reagent Kit (vl) (10X Genomics). Individual paired sequences were cloned into a single construct expressing mouse TRBC and TRAC regions to create human-mouse TCRs separated by a P2A. The TCR constructs were packaged with Lenti-X™ cells (Takara Bio USA, Mountain View, CA). Briefly, TCR constructs were mixed with packaging plasmids (pREV/pTAT/pVSVG/pGAGPOL) and incubated with jetPRIME® (Polyplus, Illkirch, France) reagent according to the manufacturer's protocol. After 24 hours, cultures were washed with Opti-Pro™ SFM medium (LifeTech). Viral supernatants were harvested 48 hours after transfection and were concentrated using Vivaspin® 20 centrifugal concentrators (Sartorius, Bohemia, NY) to desired volume (Sartorius, Bohemia, NY). Lentiviral titer was determined by GFP or TCRα/β expression using a TCR^{-/-} Jurkat cell line. To quantify viral titers, GFP expression was assessed by flow cytometric analysis 48 to 72 hours post-transduction. Samples were analyzed using a CytoFLEX™ flow cytometer (Beckman Coulter). Titer was calculated as the percentage of cells expressing GFP as TU/ml using the formula: TU/ml=% of GFP+×(number of cells used in transduction)×(dilution factor)×1000.

### (ii) T Cell Engineering

CD8+ T cells (T Cells) were isolated from leukopaks using Miltenyi MultiMACS™ Cell24 Separator (Miltenyi Biotec) and the StraightFrom® Leukopak® Human CD8 MicroBead Kit following manufacturer's instructions (Miltenyi Biotec, cat. #130-117-019). Isolated CD8 T cells of >90% purity were resuspended at 10×10⁶ cells/mL in CryoStor® CS10 (StemCell Technologies, cat. #07930, Cambridge, MA) and stored at −170C for subsequent experiments. CD8 T cells were first thawed, resuspended in RPMI-1640 Medium (ATCC, cat. #30-2001) supplemented with 10% heat-inactivated fetal bovine serum (HI-FBS) (ThermoFisher, cat. #A3840002), 1X penicillin-streptomycin (ThermoFisher, cat. #15140122), 50 IU/mL recombinant human IL-2 (Peprotech, cat. #200-02), 5 ng/uL recombinant human IL-7 (R&D, cat. #207-IL), and 5 ng/uL recombinant human IL-15 (R&D, cat. #247-ILB), and rested overnight at 37 C under 5% CO₂. The next day, the cells were activated with ImmunoCult™ human CD3/CD28 T cell activator (StemCell Technologies, cat. #10971) for 16 hrs and then transduced with individual TCRs packaged into lentivirus at 1×10⁸-1×10⁹ U/mL. 72 hours later, the residual virus was washed out and cells were pooled to create a library of TCR transduced T cells. Cells expressing the exogenous mouse-human TCR were labeled with a biotin-labeled anti-mouse TCR antibody (BioLegend, cat. #109204), and isolated using anti-biotin-conjugated microbeads (Miltenyi, cat. #130-090485) following the manufacturer's instructions. Isolated cells were expanded for seven days, resuspended in CryoStor® CS10 (StemCell Technologies, cat. #07930, Cambridge, MA) and frozen.

(iii) Peptide Library Design

To generate a cancer testis antigen (CTA) peptide library, amino acid sequences spanning the coding region of 1,600 cancer testis antigens were divided into 66-mer amino acid tiles overlapping by 20 amino acids. The tiles were synthesized on a silicon chip (Twist Bioscience) and cloned into a lentivirus expression vector. Similarly, the human genome peptide library was generated by tiling across the human genome coding sequences spanning all proteins of the human genome with 90-mer amino acid tiles.

### (iv) Library Virus Packaging and Titering

To generate peptide library-expressing reporter cells, peptide library constructs were first packaged using Lenti-X™ cells (Takara Bio USA, Mountain View, CA). Briefly, the Lenti-X™ cells were plated at 75% confluency in a CellBIND® polysyrene CellSTACK® 5-stack chamber (Corning), and transfected using jetPRIME® transfection reagent (Polyplus, Illkirch, France). Peptide libraries were mixed with packaging plasmids (pREV/pTAT/pVSVG/pGAGPOL) and incubated with the jetPRIME® reagent according to the manufacturer's protocol. Opti-Pro™ SFM medium (LifeTech) was added at 24 hours post-transfection. Viral supernatants were harvested 48 hours after transfection and were concentrated using Vivaflow® 50 cassettes (Sartorius, Bohemia, NY). Lentiviral titer was determined by puromycin colonies formation using Lenti-X™ cells using serial dilution of viral supernatant. To quantify viral titers, puromycin resistance colonies were selected 48 hours post-transduction. Puromycin resistance colonies were visualized by crystal violet staining and counted. Titer was calculated as the colony forming unit as TU/ml using the formula: TU/ml=Number of puromycin resistance colonies× dilution factor×1000.

MHC-null HEK293T cells expressing a granzyme-activated fluorescent reporter were engineered to express an MHC that presents a MAGEC2 immunogenic peptide described herein, such as HLA-B*07:02 monoallelic reporter cells, HLA-A*24:02 monoallelic reporter cells, and the like. 6×10⁷ (for CTA library) or 2.4×10⁸ (for genome wide library) monoallelic reporter cells were plated in a Falcon® 875 cm² Rectangular Straight Neck Cell Culture Multi-Flask (Corning) and transduced with the peptide library packaged lentivirus at a MOI of 5. As a positive control, cells expressing a single 90-mer amino acid tile derived from the human genome library described above that were known to be recognized by a spiked-in TCR was added to the pool of cells at a ratio of 1:40,000.

### (v) Co-Culture and Enrichment of Granzyme-Killed Cells

2.5×10⁷ Transduced CD8⁺ T cells were thawed and restimulated with irradiated PMBC feeder cells at a 1:20 T cell:PBMC ratio in media supplemented with 0.1 mg/mL anti-CD3 (OKT3, eBioscience) and 50 U/mL IL-2 (Peprotech). After 7 days, T cells were added to reporter cells transduced with the peptide library at an E:T ratio of 1:1 and incubated for 4 hours. All cells were collected by trypsinization and stained with Annexin V-conjugated microbeads (Miltenyi) according to the manufactures instructions and separated using an autoMACS® Pro separator (Miltenyi). Cells positive for the fluorescent granzyme reporter were sorted using a MoFlo® Astrios™ Cell Sorter (Beckman Coulter) and stored in DNA/RNA Shield™ (Zymo Research) for subsequent analysis.

### (vi) Next-Generation Sequencing (NGS) and Data Analysis

Genomic DNA was extracted using GeneJET™ Genomic DNA Purification Kit (Thermo Fisher Scientific, Waltham, MA) and prepared for NGS sequencing using two rounds of PCR amplification. In brief, the first round PCR amplified the peptide cassette and the second round added sequencing adapter and sample indexes before sequencing using an Illumina NextSeq™ instrument (Illumina, San Diego, CA). The proportion of mapped reads for each peptide and the fold enrichment was calculated over the peptide proportion in the input library. The geometric mean of 8 technical replicates were calculated and an enrichment of >4 fold in >2 identical sequences was considered for subsequent analysis.

b. TCR Discovery

### (i) Peptide Prediction and Target Verification

Overlapping peptide sequences with a score>4 fold were used to predict MHC binding with NetMHC4.0 (available on the World Wide Web at cbs.dtu.dk) and the top predicted MHC binding peptides were synthesized (Genscript). Monoallelic HEK293T reporter cells were pulsed with 1 uM of each peptide candidate for 1 hour and then co-cultured with the pool of TCR-transduced T cells at an E:T ratio of 1:1. After 24 hours, the cultures were transferred to V-bottom 96-well plates, centrifuged at 2,000 rpm for 2 minutes, and the supernatants were assayed for IFNγ using Ella 3^{rd} generation IFNγ cartridges (ProteinSimple) according to the manufacturer's instructions.

### (ii) Identification of the Corresponding TCR

To identify the corresponding TCR from the pool of TCR-transduced T cells, MHC monoallelic HEK293T cells were peptide-pulsed for 1 hour and co-cultured with the pool of T cells at an E:T ratio of 1:1 for 16-24 hours. The T cells were mixed, collected by pipetting, and labeled with a CD137 microbead kit (Miltenyi) following the manufacturer's instructions. In brief, T cells were first stained with PE-labeled anti-CD137 and AF647-labeled anti-CD69 (BioLegend), washed, and then labeled with anti-PE microbeads. Labeled cells were enriched with an autoMACS® Pro separator (Miltenyi) and cells positive for the granyme reporter were sorted on a MoFlo® Astrios™ Cell Sorter (Beckman Coulter). Genomic DNA was extracted from the sorted cells and the TCR expression cassette was PCR amplified and prepared for next generation sequencing (NGS).

c. TCR Characterization

### (i) T Cell Recognition Assay

To characterize the recognition of the identified peptide by a TCR, wild-type HEK293 cells were pulsed with serial dilutions of peptide and co-cultured with T cells transduced with the TCR of interest. After 24 hours, cultures were mixed by pipetting, and centrifuged at 2,000 rpm for 2 minutes. Supernatants were collected and measured for IFNγ secretion using a human IFNγ 3rd generation single-plex assay (Protein Simple) following the manufacturer's instructions. Alternatively, monoallelic HEK293T cells expressing a granzyme-activated fluorescent reporter were pulsed with serial dilutions of peptide and co-cultured with T cells transduced with an individual TCR of interest. After 4 hours, the cultures were harvested by pipetting and fluorescence of the reporter was detected using a CytoFLEX™ S instrument (Beckman Coulter).

To detect reactivity to melanoma cell lines, wild-type melanoma cell lines were seeded at 6×10⁴ cells in a 96-well flat bottom plate and rested for 16 hours at 37° C. in 5% CO₂. On the following day, T cells were thawed, washed in complete RPMI-1640 medium, and added to the wells for an effector to target ratio of 1:1. The co-culture was incubated for 16 hours. Cells were then harvested by pipetting, transferred to V-bottom 96-well plates, and centrifuged at 2,000 rpm for 2 minutes. The supernatant was collected for IFNγ measurement using a human IFNγ 3rd generation single-plex assay (Protein Simple). For flow cytometry analysis (Cytoflex S™ instrument, Beckman Coulter), cell pellets were washed with FACS buffer (PBS, 0.5% BSA, 2 mM EDTA) and stained for activation-induced markers, including PE-conjugated anti-CD137 (Miltenyi), AF647-conjugated anti-CD69 (Biolegend), and BV421-conjugated anti-CD8 (BioLegend).

### (ii) Cancer Cell Culture and Incucyte® NucLight™ Red Transduction

Melanoma lines were purchased from ATCC (Manassas, VA) and HEK293T cells were originally from Takara Bio (Shiga, Japan). All media were supplemented with 10% FBS and 1% penicillin and incubated at 37° C. in 5% CO₂. A101D, A2058, HT144, and HEK293T cells were maintained in complete DMEM. SK-MEL-5 was cultured in complete EMEM, whereas HMCB required complete EMEM with 10 mM HEPES. Untransduced cancer cells were maintained in preparation for the T cell recognition assay, and 10⁶ cells of each cell line were introduced with Incucyte® NucLight™ Red lentivirus reagent (Sartorius, Gottingen, Germany). Positively-labeled cells were selected using 1.5 ug/mL of puromycin over 72 hours.

(iii) Incucyte® Cytotoxicity Assay

Melanoma cells selected for Incucyte® NucLight™ analysis were seeded into 96-well flat bottom plates (Corning, Flintshire, UK) at 10⁴ cells, except for HMCB cells, which were plated at 5×10³ cells. On the same day, T cells were thawed, washed in complete RPMI-1640, and allowed to rest overnight in medium with 50 U/mL IL-2 (PeproTech), 5 ng/mL IL-7, and 5 ng/mL IL-15 (R&D Systems). All cells were rested for 16 hours at 37° C. in 5% CO₂. A two-fold serial dilution of the T cells was performed for the effector to target ratios, with the highest E:T at 4:1, and the lowest at 1:2. One image per whole well from two technical replicates was taken every 2 hours over a 72 hour time course. Imaging utilized a 4× objective lens and data were analyzed using the Incucyte® Basic Software. Red channel acquisition time was 400 ms, and its background was subtracted by enabling Top-Hat segmentation. Edge split was applied so that the software can recognize closely spaced cells as multiple objects, rather than one object, if the setting were absent.

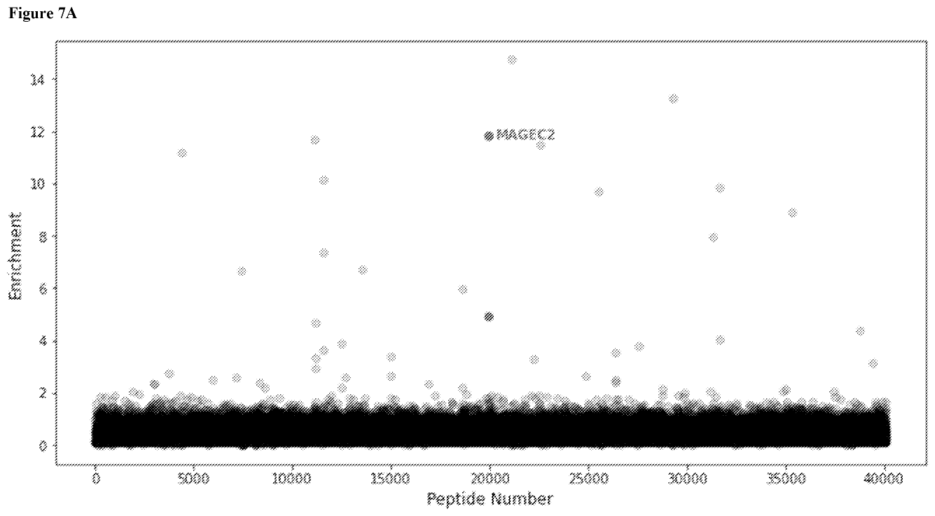

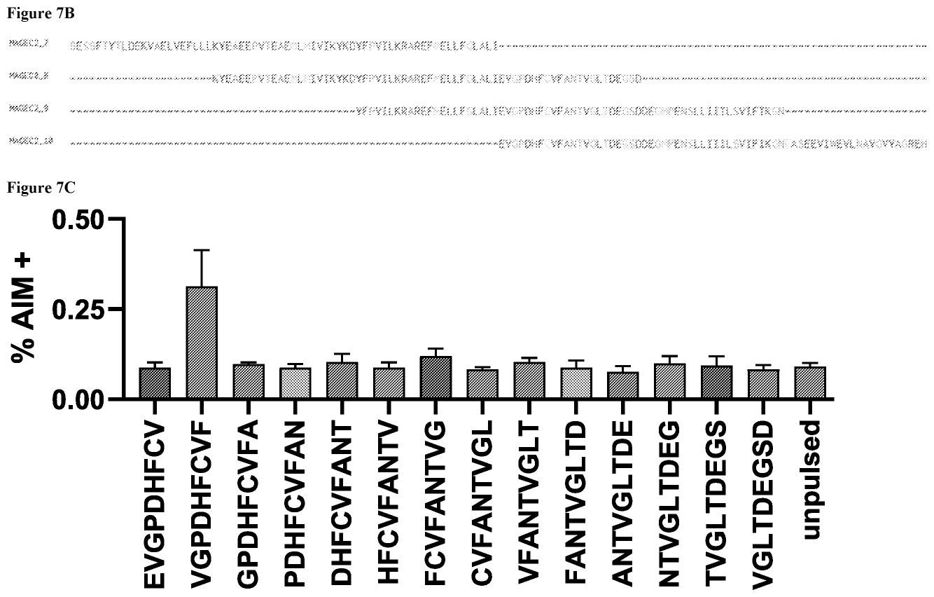

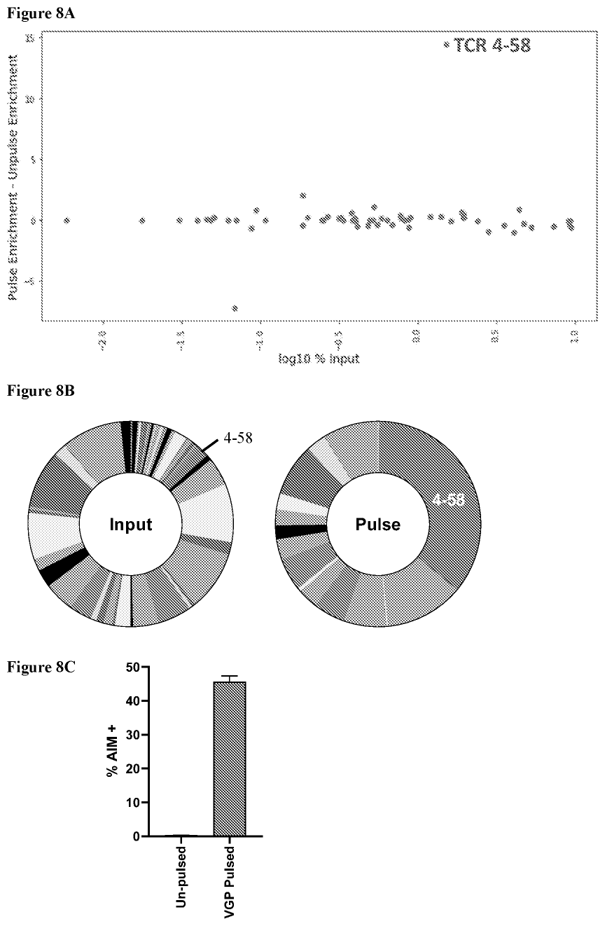

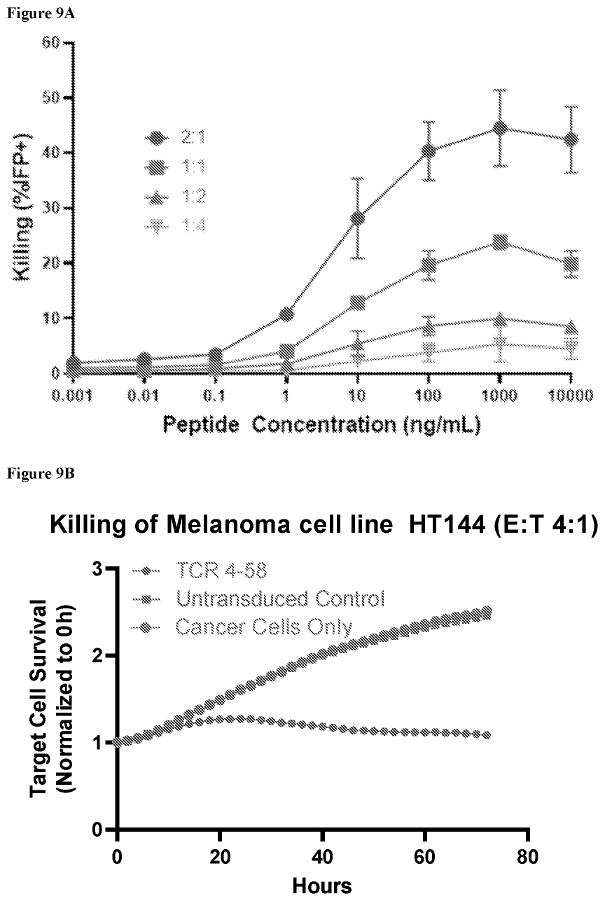

Data shown in FIGS. 7-9 were generated according to similar materials, methods, and experiments as described above.

### Example 2: Identification of MAGEC2 Immunogenic Epitopes and Binding Proteins Thereto

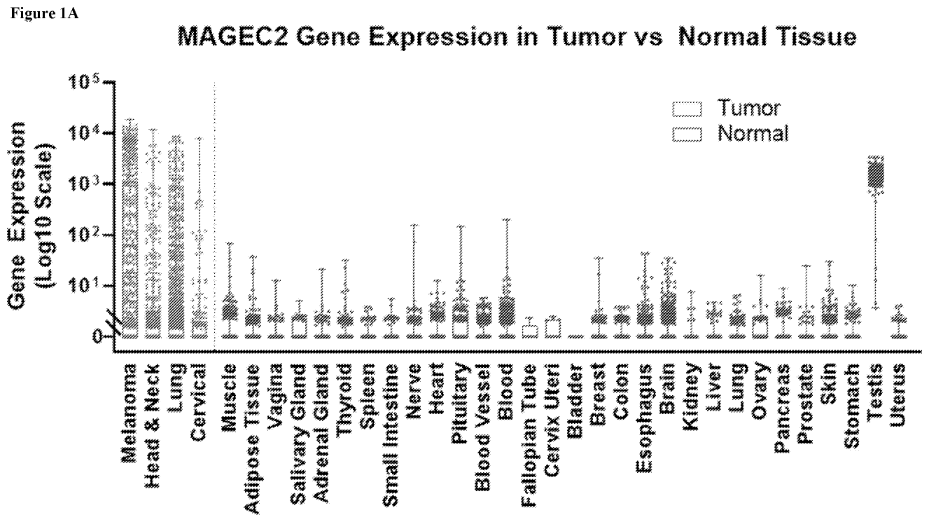

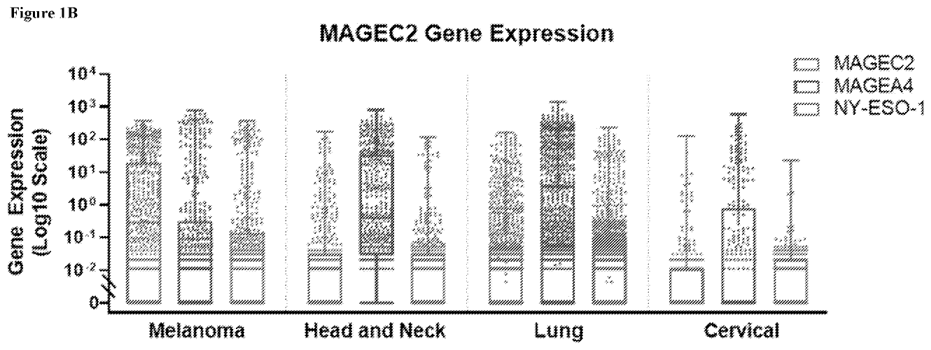

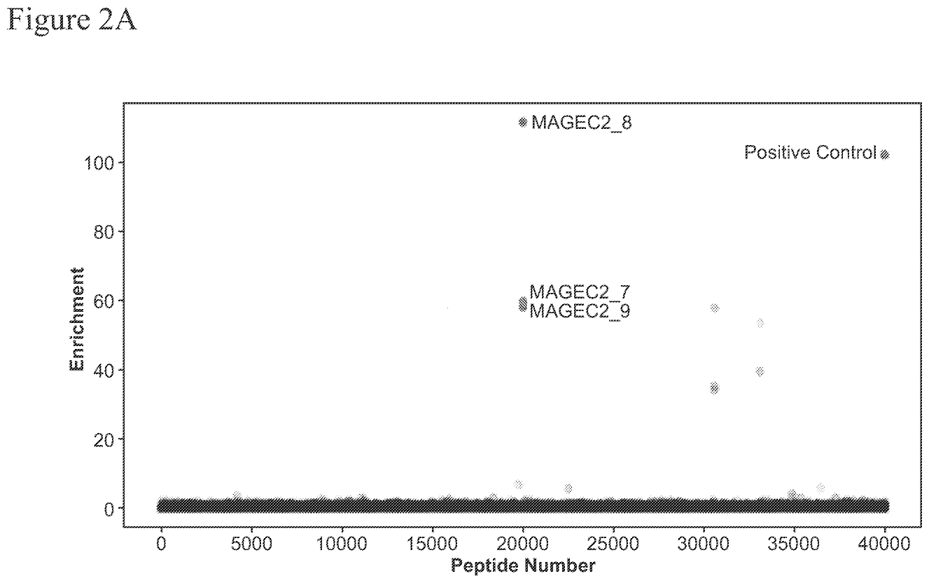

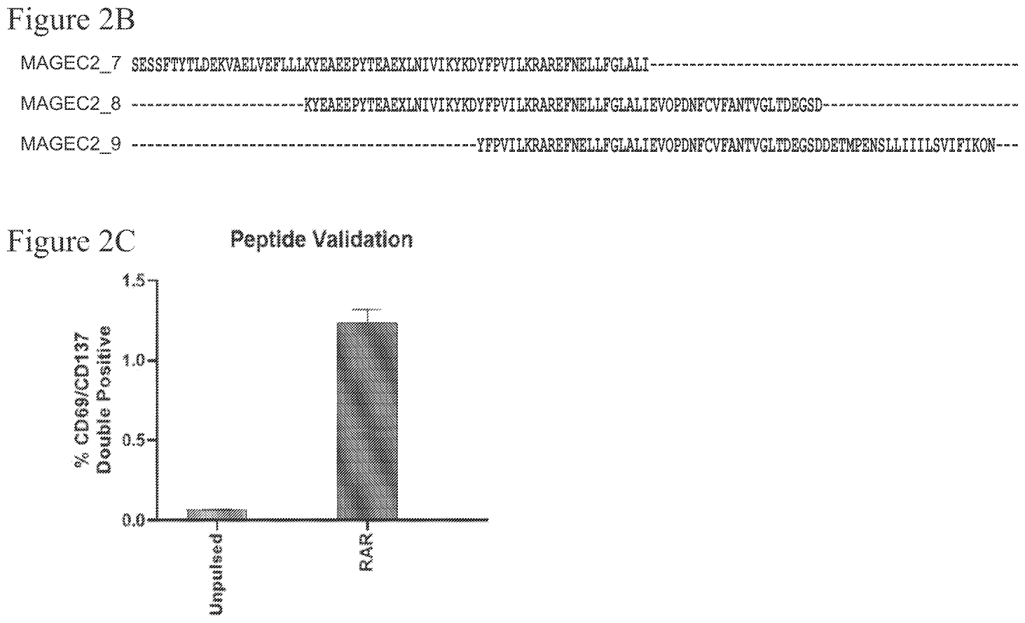

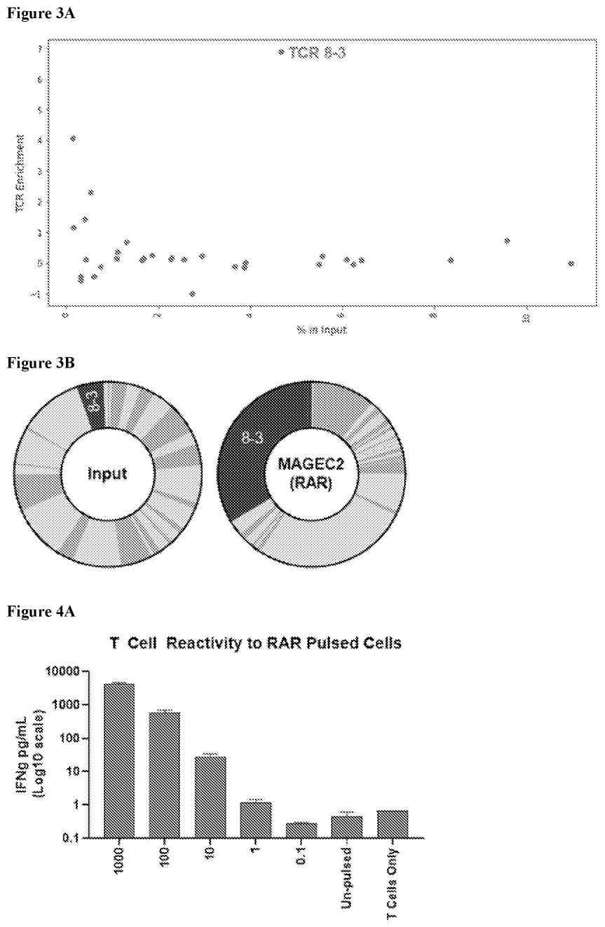

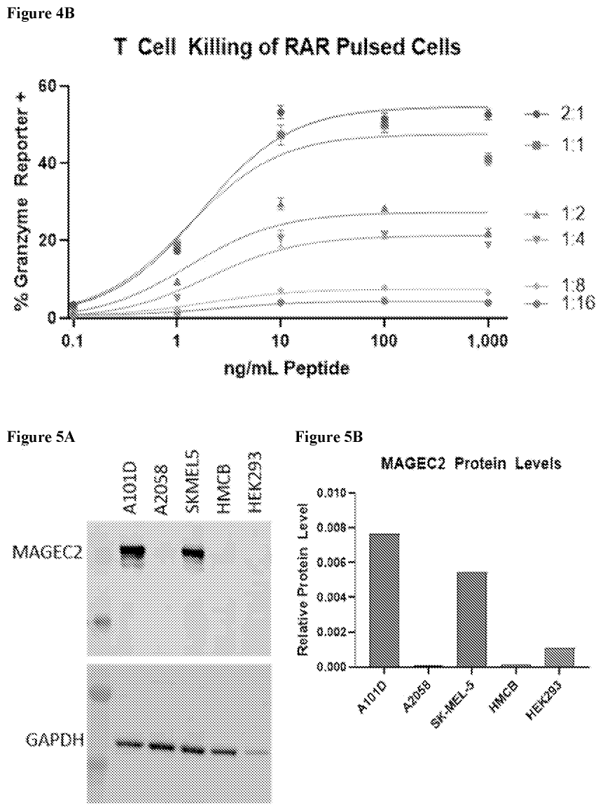

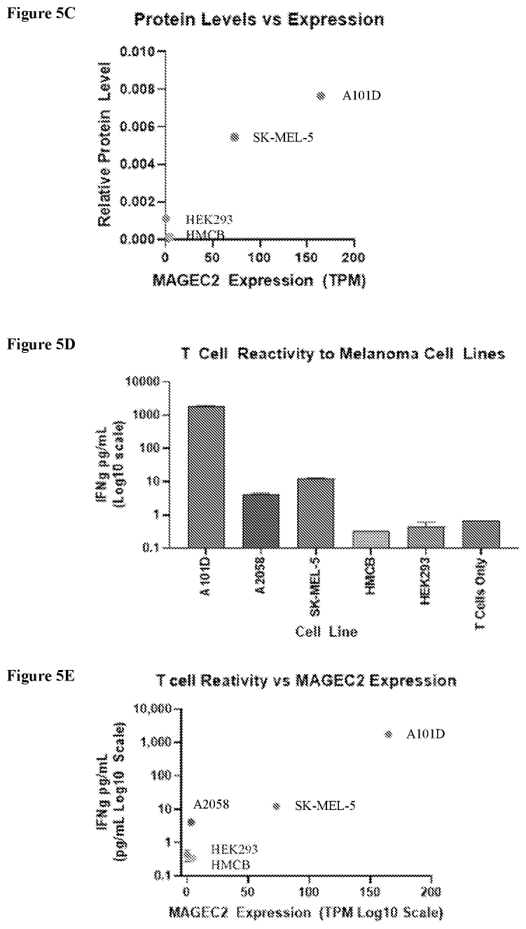

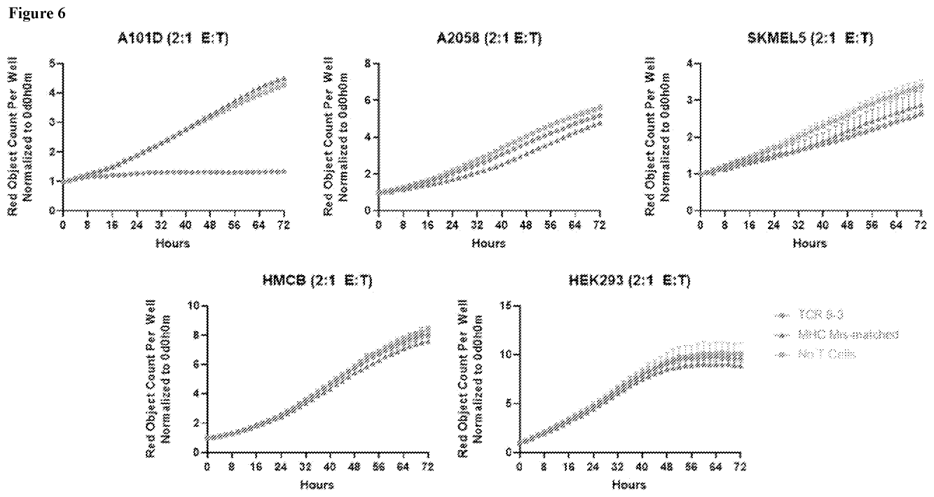

A high-throughput antigen discovery platform that enables rapid identification of TCRs from a pool of TCRs obtained from an adoptive cell therapy product used to treat autologous patients with melanoma that recognize immunogenic peptides presented by MHC molecules was developed and applied to identify the recognized immunogenic peptides (see Example 1). Antigenic peptides of MAGEC2, which is a gene whose expression is associated with certain disorders like cancer and not normally expressed in tissues outside of testis (FIG. 1), were identified as being recognized in presentation by HLA-B*07:02 (FIG. 2A). Common sequences shared by overlapping tiles of the searched sequence library and bioinformatics analyses allowed for the identification of immunogenic MAGEC2 peptide sequences (FIG. 2B; see also FIG. 2C for a representative immunogenic peptide validation named “RAR”). The RAR representative immunogenic peptide was then used to screen pools of TCR-transduced T cells and hits were identified, such as TCR 8-3 (FIGS. 3A and 3B). The representative TCR 8-3 was demonstrated to kill cells expressing RAR peptide-HLA-B*07:02 (pMHC) complex in peptide pulse-based cytotoxicity assays (FIGS. 4A and 4B). The representative TCR 8-3 was further demonstrated to kill cancer cells that express MAGEC2, including melanoma cells expressing MAGEC2 at varying levels (FIGS. 5A-5E and 6). Similar experiments were performed to identify additional immunogenic MAGEC2 peptide sequences presented by HLA-A*24 serotypes, such as HLA-A*24:02, and TCRs that recognize such peptide-HLA complexes (see FIGS. 7-9).

Thus, physiologically relevant TCR-antigen pairs in the context of MHC molecules have been identified.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS AND SCOPE

The details of one or more embodiments encompassed by the present invention are set forth in the description above. Although representative, exemplary materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which the present invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

What is claimed is:

1. An immunogenic peptide comprising a peptide epitope selected from peptide sequences listed in Table 1.

2. An immunogenic peptide consisting of a peptide epitope selected from peptide sequences listed in Table 1.

3. The immunogenic peptide of claim 1 or 2, wherein the immunogenic peptide is derived from a MAGEC2 protein, optionally wherein the immunogenic peptide is 8, 9, 10, 11, 12, 13, 14, 15, 16, 17, 18, 19, 20, 21, 22, 23, 24, or 25 amino acids in length.

4. The immunogenic peptide of any one of claims 1-3, wherein the immunogenic peptide is capable of eliciting an immune response against MAGEC2 and/or MAGEC2-expressing cells in a subject, optionally wherein the immune response is i) a T cell response and/or a CD8+ T cell response and/or ii) selected from the group consisting of T cell expansion, cytokine release, and/or cytotoxic killing.

5. An immunogenic composition comprising at least one immunogenic peptide according to any one of claims 1-4.

6. The immunogenic composition of claim 5, further comprising an adjuvant.

7. The immunogenic composition of claim 5 or 6, wherein the immunogenic composition is capable of eliciting an immune response against MAGEC2 and/or MAGEC2-expressing cells in a subject, optionally wherein the immune response is i) a T cell response and/or a CD8+ T cell response and/or ii) selected from the group consisting of T cell expansion, cytokine release, and/or cytotoxic killing.

8. A composition comprising a peptide epitope selected from peptide sequences listed in Table 1, and an MHC molecule.

9. The composition of claim 8, wherein the MHC molecule is an MHC multimer, optionally wherein the MHC multimer is a tetramer.

10. The composition of claim 8 or 9, wherein the MHC molecule is an MHC class I molecule.

11. The composition of any one of claims 9-10, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

12. A stable MHC-peptide complex, comprising an immunogenic peptide according to any one of claims 1-4 in the context of an MHC molecule.

13. The stable MHC-peptide complex of claim 12, wherein the MHC molecule is an MHC multimer, optionally wherein the MHC multimer is a tetramer.

14. The stable MHC-peptide complex of claim 12 or 13, wherein the MHC molecule is an MHC class I molecule.

15. The stable MHC-peptide complex of any one of claims 12-14, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

16. The stable MHC-peptide complex of any one of claims 12-15, wherein the peptide epitope and the MHC molecule are covalently linked and/or wherein the alpha and beta chains of the MHC molecule are covalently linked.

17. The stable MHC-peptide complex of any one of claims 12-16, wherein the stable MHC-peptide complex comprises a detectable label, optionally wherein the detectable label is a fluorophore.

18. An immunogenic composition comprising the stable MHC-peptide complex according to any one of claims 12-17, and an adjuvant.

19. An isolated nucleic acid that encodes the immunogenic peptide of according to any one of claims 1-4, or a complement thereof.

20. A vector comprising the isolated nucleic acid of claim 19.

21. A cell that a) comprises the isolated nucleic acid of claim 19, b) comprises the vector of claim 20, and/or c) produces one or more immunogenic peptides according to any one of claims 1-4 and/or presents at the cell surface one or more stable MHC-peptide complexes according to any one of claims 12-17, optionally wherein the cell is genetically engineered.

22. A device or kit comprising a) one or more immunogenic peptides according to any one of claims 1-4 and/or b) one or more stable MHC-peptide complexes according to any one of claims 12-17, said device or kit optionally comprising a reagent to detect binding of a) and/or b) to a binding protein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

23. A method of detecting T cells that bind a stable MHC-peptide complex comprising:
a) contacting a sample comprising T cells with a stable MHC-peptide complex according to any one of claims 12-17; and
b) detecting binding of T cells to the stable MHC-peptide complex, optionally further determining the percentage of stable MHC-peptide-specific T cells that bind to the stable MHC-peptide complex, optionally wherein the sample comprises peripheral blood mononuclear cells (PBMCs).

24. The method of claim 23, wherein the T cells are CD8+ T cells.

25. The method of any one of claims 22-24, wherein the detecting and/or determining is performed using fluorescence activated cell sorting (FACS), enzyme linked immunosorbent assay (ELISA), radioimmune assay (RIA), immunochemically, Western blot, or intracellular flow assay.

26. The method of any one of claims 22-25, wherein the sample comprises T cells contacted with, or suspected of having been contacted with, one or more MAGEC2 proteins or fragments thereof.

27. A method of determining whether a T cell has had exposure to MAGEC2 comprising:
a) incubating a cell population comprising T cells with an immunogenic peptide according to any one of claims 1-4 or a stable MHC-peptide complex according to any one of claims 12-17; and
b) detecting the presence or level of reactivity,
wherein the presence of or a higher level of reactivity compared to a control level indicates that the T cell has had exposure to MAGEC2, optionally wherein the cell population comprising T cells is obtained from a subject.

28. A method for predicting the clinical outcome of a subject afflicted with a disorder characterized by MAGEC2 expression comprising:
a) determining the presence or level of reactivity between T cells obtained from the subject and one more immunogenic peptides according to any one of claims 1-4 or one or more stable MHC-peptide complexes according to any one of claims 12-17; and
b) comparing the presence or level of reactivity to that from a control, wherein the control is obtained from a subject having a good clinical outcome,
wherein the presence or a higher level of reactivity in the subject sample as compared to the control indicates that the subject has a good clinical outcome.

29. A method of assessing the efficacy of a therapy for a disorder characterized by MAGEC2 expression comprising:
a) determining the presence or level of reactivity between T cells obtained from the subject and one more immunogenic peptides according to any one of claims 1-4 or one or more stable MHC-peptide complexes according to any one of claims 12-17, in a first sample obtained from the subject prior to providing at least a portion of the therapy to the subject, and
b) determining the presence or level of reactivity between the one more immunogenic peptides according to any one of claims 1-4, or the one or more stable MHC-peptide complexes according to any one of claims 12-17, and T cells obtained from the subject present in a second sample obtained from the subject following provision of the therapy to the subject,
wherein the presence or a higher level of reactivity in the second sample, relative to the first sample, is an indication that the therapy is efficacious for treating the disorder characterized by MAGEC2 expression in the subject.

30. The method of any one of claims 27-29, wherein the level of reactivity is indicated by a) the presence of binding and/or b) T cell activation and/or effector function, optionally wherein the T cell activation or effector function is T cell proliferation, killing, or cytokine release.

31. The method of any one of claims 27-30, further comprising repeating steps a) and b) at a subsequent point in time, optionally wherein the subject has undergone treatment to ameliorate the disorder characterized by MAGEC2 expression between the first point in time and the subsequent point in time.

32. The method of any one of claims 27-31, wherein the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

33. The method of any one of claims 27-32, wherein the control level is a reference number.

34. The method of any one of claims 27-33, wherein the control level is a level of a subject without the disorder characterized by MAGEC2 expression.

35. A method of preventing and/or treating a disorder characterized by MAGEC2 expression in a subject comprising administering to the subject a therapeutically effective amount of a composition according to any one of claims 1-22.

36. A method of identifying a peptide-binding molecule, or antigen-binding fragment thereof, that binds to a peptide epitope selected from the peptide sequences listed in Table 1 comprising:
a) providing a cell presenting a peptide epitope selected from the peptide sequences listed in Table 1 in the context of an MHC molecule on the surface of the cell;
b) determining binding of a plurality of candidate peptide-binding molecules or antigen-binding fragments thereof to the peptide epitope in the context of the MHC molecule on the cell; and
c) identifying one or more peptide-binding molecules or antigen-binding fragments thereof that bind to the peptide epitope in the context of the MHC molecule.

37. The method of claim 36, wherein the step a) comprises contacting the MHC molecule on the surface of the cell with a peptide epitope selected from the peptide sequences listed in Table 1.

38. The method of claim 36, wherein the step a) comprises expressing the peptide epitope selected from the peptide sequences listed in Table 1 in the cell using a vector comprising a heterologous sequence encoding the peptide epitope.

39. A method of identifying a peptide-binding molecule or antigen-binding fragment thereof that binds to a peptide epitope selected from the peptide sequences listed in Table 1 comprising:
a) providing a peptide epitope either alone or in a stable MHC-peptide complex, comprising a peptide epitope selected from the peptide sequences listed in Table 1, either alone or in the context of an MHC molecule;
b) determining binding of a plurality of candidate peptide-binding molecules or antigen-binding fragments thereof to the peptide or stable MHC-peptide complex; and
c) identifying one or more peptide-binding molecules or antigen-binding fragments thereof that bind to the peptide epitope or the stable MHC-peptide complex, optionally wherein the MHC or MHC-peptide complex is as according to any one of claims 8-17.

40. The method of claim 39, wherein the plurality of candidate peptide binding molecules comprises an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

41. The method of claim 39 or 40, wherein the plurality of candidate peptide binding molecules comprises at least 2, 5, 10, 100, 10³, 10⁴, 10⁵, 10⁶, 10⁷, 10⁸, 10⁹, or more, different candidate peptide binding molecules.

42. The method of any one of claims 39-41, wherein the plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject.

43. The method of claim 42, wherein the subject or population of subjects are a) not afflicted with a disorder characterized by MAGEC2 expression and/or have recovered from a disorder characterized by MAGEC2 expression, or b) are afflicted with a disorder characterized by MAGEC2 expression.

44. The method of claim 42 or 43, wherein the subject or population of subjects has been administered a composition according to any one of claims 1-22.

45. The method of any one of claims 42-44, wherein the subject is an animal model of a disorder characterized by MAGEC2 expression and/or a mammal, optionally wherein the mammal is a human, a primate, or a rodent.

46. The method of any one of claims 42-45, wherein the subject is an animal model of a disorder characterized by MAGEC2 expression, an HLA-transgenic mouse, and/or a human TCR transgenic mouse.

47. The method of any one of claims 42-46, wherein the sample comprises peripheral blood mononuclear cells (PBMCs), T cells, and/or CD8+ memory T cells.

48. The peptide-binding molecule or antigen-binding fragment thereof identified according to any one of claims 39-47, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

49. A method of treating a disorder characterized by MAGEC2 expression in a subject comprising administering to the subject a therapeutically effective amount of genetically engineered T cells that express a peptide-binding molecule or antigen-binding fragment thereof that i) binds to a peptide epitope selected from the sequences listed in Table 1, ii) is identified according to the method according to any one of claims 39-48, and/or iii) binds to a stable MHC-peptide complex comprising a peptide epitopes selected from the sequences listed in Table 1 in the context of an MHC molecule, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, optionally wherein the MHC or MHC-peptide complex is as according to any one of claims 8-17.

50. The method of claim 49, wherein the T cells are isolated from a) the subject, b) a donor not afflicted with the disorder characterized by MAGEC2 expression, or c) a donor recovered from a disorder characterized by MAGEC2 expression.

51. A method of treating a disorder characterized by MAGEC2 expression in a subject comprising transfusing antigen-specific T cells to the subject, wherein the antigen-specific T cells are generated by:
a) stimulating immune cells from a subject with a composition according to any one of claims 1-22; and
b) expanding antigen-specific T cells in vitro or ex vivo, optionally i) isolating immune cells from the subject before stimulating the immune cells and/or ii) wherein the immune cells comprise PBMCs, T cells, CD8+ T cells, naive T cells, central memory T cells, and/or effector memory T cells.

52. The method of claim 51, wherein the agents are placed in contact under conditions and for a time suitable for the formation of at least one immune complex between the peptide epitope, immunogenic peptide, stable MHC-peptide complex, T cell receptor, and/or immune cells.

53. The method of claim 51 or 52, wherein the peptide epitope, immunogenic peptide, stable MHC-peptide complex, and/or T cell receptor are expressed by cells and the cells are expanded and/or isolated during one or more steps.

54. The method of any one of claims 23-53, wherein the disorder characterized by MAGEC2 expression is a cancer or relapse thereof, optionally wherein the cancer is selected from the group consisting of melanoma, head & neck cancer, lung cancer, cervical cancer, prostate cancer, multiple myeloma, hepatocellular carcinoma, breast invasive carcinoma, and bladder urothelial carcinoma.

55. The method of any one of claims 23-54, wherein the subject is an animal model of a disorder characterized by MAGEC2 expression and/or a mammal, optionally wherein the mammal is a human, a primate, or a rodent.

56. A binding protein that binds a polypeptide comprising an immunogenic peptide sequence according to any one of claims 1-4, an immunogenic peptide according to any one of claims 1-4, and/or the stable MHC-peptide complex according to any one of claims 12-17, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

57. The binding protein of claim 56 comprising:
a) a T cell receptor (TCR) alpha chain CDR sequence with at least about 80% identity to a TCR alpha chain CDR sequence selected from the group consisting of TCR alpha chain CDR sequences listed in Table 2; and/or
b) a TCR beta chain CDR sequence with at least about 80% identity to a TCR beta chain CDR sequence selected from the group consisting of TCR beta chain CDR sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M.

58. The binding protein of claim 56 comprising:
a) a TCR alpha chain variable (V_{α}) domain sequence with at least about 80% identity to a TCR V_{α} domain sequence selected from the group consisting of TCR V_{α} domain sequences listed in Table 2; and/or
b) a TCR beta chain variable (V_{β}) domain sequence with at least about 80% identity to a TCR V_{β} domain sequence selected from the group consisting of TCR V_{β} domain sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M.

59. The binding protein of claim 56 comprising:
a) a TCR alpha chain sequence with at least about 80% identity to a TCR alpha chain sequence selected from the group consisting of TCR alpha chain sequences listed in Table 2; and/or
b) a TCR beta chain sequence with at least about 80% identity to a TCR beta chain sequence selected from the group consisting of TCR beta chain sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M.

60. The binding protein of claim 56 comprising:
a) a TCR alpha chain CDR sequence selected from the group consisting of TCR alpha chain CDR sequences listed in Table 2; and/or
b) a TCR beta chain CDR sequence selected from the group consisting of TCR beta chain CDR sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M.

61. The binding protein of claim 56 comprising:
a) a TCR alpha chain variable (V_{α}) domain sequence selected from the group consisting of TCR V_{α} domain sequences listed in Table 2; and/or
b) a TCR beta chain variable (V_{β}) domain sequence selected from the group consisting of TCR V_{β} domain sequences listed in Table 2, wherein the binding protein is capable of binding to a MAGEC2 immunogenic peptide-MHC (pMHC) complex,
optionally wherein the binding affinity has a K_{d} less than or equal to about 5×10⁻⁴ M.

62. The binding protein of claim 56 comprising:
a) a TCR alpha chain sequence selected from the group consisting of TCR alpha chain sequences listed in Table 2; and/or
b) a TCR beta chain sequence selected from the group consisting of TCR beta chain sequences listed in Table 2, wherein the binding protein is capable of binding to a MAGEC2 immunogenic peptide-MHC (pMHC) complex, optionally wherein the binding affinity has a K_{d} less than or equal to about 5×10⁻⁴ M.

63. The binding protein of any one of claims 56-62, wherein 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2.

64. The binding protein of any one of claims 56-63, wherein the binding protein is chimeric, humanized, or human.

65. The binding protein of any one of claims 56-64, wherein the binding protein comprises a binding domain having a transmembrane domain, and an effector domain that is intracellular.

66. The binding protein of any one of claims 56-65, wherein the TCR alpha chain and the TCR beta chain are covalently linked, optionally wherein the TCR alpha chain and the TCR beta chain are covalently linked through a linker peptide.

67. The binding protein of any one of claims 56-66, wherein the TCR alpha chain and/or the TCR beta chain are covalently linked to a moiety, optionally wherein the covalently linked moiety comprises an affinity tag or a label.

68. The binding protein of claim 67, wherein the affinity tag is selected from the group consisting of aCD34 enrichment tag, glutathione-S-transferase (GST), calmodulin binding protein (CBP), protein C tag, Myc tag, HaloTag, HA tag, Flag tag, His tag, biotin tag, and V5 tag, and/or wherein the label is a fluorescent protein.

69. The binding protein of any one of claims 56-68, wherein the covalently linked moiety is selected from the group consisting of an inflammatory agent, cytokine, toxin, cytotoxic molecule, radioactive isotope, or antibody or antigen-binding fragment thereof.

70. The binding protein of any one of claims 56-69, wherein the binding protein binds to the pMHC complex on a cell surface.

71. The binding protein of any one of claims 56-70, wherein the MHC or MHC-peptide complex is as according to any one of claims 8-17.

72. The binding protein of any one of claims 56-71, wherein binding of the binding protein to the MAGEC2 peptide-MHC (pMHC) complex elicits an immune response, optionally wherein the immune response is i) a T cell response and/or a CD8⁺ T cell response and/or ii) selected from the group consisting of T cell expansion, cytokine release, and/or cytotoxic killing.

73. The binding protein of any one of claims 56-72, wherein the binding protein is capable of specifically and/or selectively binding to the MAGEC2 immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M.

74. The binding protein of any one of claims 56-73, wherein the binding protein has a higher binding affinity to the peptide-MHC (pMHC) than does a known T-cell receptor, optionally wherein the higher binding affinity is at least 1.05-fold higher.

75. The binding protein of any one of claims 56-74, wherein the binding protein induces higher T cell expansion, cytokine release, and/or cytotoxic killing than does a known T-cell receptor when contacted with target cells with a heterozygous expression of MAGEC2, optionally wherein the induction is at least 1.05-fold higher.

76. The binding protein of claim 75, wherein the cytotoxic killing is a target cancer cell.

77. The binding protein of claim 76, wherein the cancer is selected from the group consisting of melanoma, head & neck cancer, lung cancer, cervical cancer, prostate cancer, multiple myeloma, hepatocellular carcinoma, breast invasive carcinoma, and bladder urothelial carcinoma.

78. The binding protein of any one of claims 56-77, wherein the binding protein does not bind to a peptide-MHC (pMHC) complex selected from the group consisting of ALKDVEERV/HLA-A*02, LLFGLALIEV/HLA-A*02, SESIKKKVL/HLA-B*44, and ASSTLYLVF/HLA-B*57.

79. A TCR alpha chain and/or beta chain selected from the group consisting of TCR alpha chain and beta chain sequences listed in Table 2.

80. An isolated nucleic acid molecule i) that hybridizes, under stringent conditions, with the complement of a nucleic acid encoding a polypeptide selected from the group consisting of polypeptide sequences listed in Table 2, ii) a sequence with at least about 80% homology to a nucleic acid encoding a polypeptide selected from the group consisting of the polypeptide sequences listed in Table 2, and/or iii) ii) a sequence with at least about 80% homology to a nucleic acid encoding listed in Table 2, optionally wherein the isolated nucleic acid molecule comprises 1) a TRAV, TRAJ, and/or TRAC gene or fragment thereof selected from the group of TRAV, TRAJ, and TRAC genes listed in Table 2 and/or 2) a TRBV, TRBJ, and/or TRBC gene or fragment thereof selected from the group of TRBV, TRBJ, and TRBC genes listed in Table 2.

81. The isolated nucleic acid of claim 80, wherein the nucleic acid is codon optimized for expression in a host cell.

82. A vector comprising the isolated nucleic acid of claim 80 or 81, optionally wherein i) the vector is a cloning vector, expression vector, or viral vector and/or ii) the vector comprises a vector sequence listed in Table 3.

83. The vector of claim 82, wherein the vector further comprises a nucleic acid sequence encoding CD8α and/or CD8β.

84. The vector of claim 83, wherein the nucleic acid sequence encoding CD8α or CD8β is operably linked to a nucleic acid encoding a tag.

85. The vector of claim 83 or 84, wherein the nucleic acid encoding a tag is at the 5′ upstream of the nucleic acid sequence encoding CD8α or CD8β such that the tag is fused to the N-terminus of CD8α or CD8β.

86. The vector of claim 84 or 85, wherein the tag is a CD34 enrichment tag.

87. The vector of any one of claims 83-86, wherein the isolated nucleic acid of claim 80 or 81, and the nucleic acid sequence encoding CD8α and/or CD8β are interconnected with an internal ribosome entry site or a nucleic acid sequence encoding a self-cleaving peptide.

88. The vector of claim 87, wherein the self-cleaving peptide is P2A, E2A, F2A or T2A.

89. A host cell which comprises the isolated nucleic acid of claim 80 or 81, comprises the vector according to any one of claims 82-88, and/or expresses the binding protein according to any one of claims 56-78, optionally wherein the cell is genetically engineered.

90. The host cell of claim 89, wherein the host cell comprises a chromosomal gene knockout of a TCR gene, an HLA gene, or both.

91. The host cell of claim 89 or 90, wherein the host cell comprises a knockout of an HLA gene selected from an α1 macroglobulin gene, α2 macroglobulin gene, α3 macroglobulin gene, β1 microglobulin gene, β2 microglobulin gene, and combinations thereof.

92. The host cell of any one of claims 89-91, wherein the host cell comprises a knockout of a TCR gene selected from a TCR α variable region gene, TCR β variable region gene, TCR constant region gene, and combinations thereof.

93. The host cell of any one of claims 89-92, wherein the host cell expresses CD8α and/or CD8β, optionally wherein the CD8α and/or CD8β is fused to a CD34 enrichment tag.

94. The host cell of claim 93, wherein host cells are enriched using the CD34 enrichment tag.

95. The host cell of any one of claims 89-94, wherein the host cell is a hematopoietic progenitor cell, peripheral blood mononuclear cell (PBMC), cord blood cell, or immune cell.

96. The host cell of claim 95, wherein the immune cell is a T cell, cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or a combination thereof.

97. The host cell of any one of claims 89-96, wherein the T cell is a naive T cell, central memory T cell, effector memory T cell, or a combination thereof.

98. The host cell of any one of claims 89-97, wherein the T cell is a primary T cell or a cell of a T cell line.

99. The host cell of any one of claims 89-98, wherein the T cell does not express or has a lower surface expression of an endogenous TCR.

100. The host cell of any one of claims 89-99, wherein the host cell is capable of producing a cytokine or a cytotoxic molecule when contacted with a target cell that comprises a peptide-MHC (pMHC) complex comprising a MAGEC2 peptide epitope in the context of an MHC molecule.

101. The host cell of claim 100, wherein the host cell is contacted with the target cell in vitro, ex vivo, or in vivo.

102. The host cell of claim 100 or 101, wherein the cytokine is TNF-α, IL-2, and/or IFN-γ.

103. The host cell of any one of claims 89-102, wherein the cytotoxic molecule is perforins and/or granzymes, optionally wherein the cytotoxic molecule is granzyme B.

104. The host cell of any one of claims 89-103, wherein the host cell is capable of producing a higher level of cytokine or a cytotoxic molecule when contacted with a target cell with a heterozygous expression of MAGEC2.

105. The host cell of claim 104, wherein the host cell is capable of producing an at least 1.05-fold higher level of cytokine or a cytotoxic molecule.

106. The host cell of any one of claims 89-103 wherein the host cell is capable of killing a target cell that comprises a peptide-MHC (pMHC) complex comprising the MAGEC2 peptide epitope in the context of an MHC molecule.

107. The host cell of claim 106, wherein the killing is determined by a killing assay.

108. The host cell of claim 106 or 107, wherein the ratio of the host cell and the target cell in the killing assay is from 20:1 to 1:4.

109. The host cell of any one of claims 106-108, wherein the target cell is a target cell pulsed with 1 μg/mL to 50 μg/mL of MAGEC2 peptide, optionally wherein the target cell is a cell monoallelic for an MHC matched to the MAGEC2 peptide.

110. The host cell of any one of claims 106-109, wherein the host cell is capable of killing a higher number of target cells when contacted with target cells with a heterozygous expression of MAGEC2, optionally wherein the cell killing is at least 1.05-fold higher.

111. The host cell of any one of claims 89-110, wherein the target cell is cell line or a primary cell, optionally wherein the target cell is selected from the group consisting of a HEK293 derived cell line, a cancer cell line, a primary cancer cell, a transformed cell line, and an immortalized cell line.

112. The host cell of any one of claims 89-111, wherein the MAGEC2 immunogenic peptide is as according to any one of claims 1-4 and/or wherein the MHC or MHC-peptide complex is as according to any one of claims 8-17.

113. The host cell of any one of claims 89-112, wherein the host cell does not induce T cell expansion, cytokine release, or cytotoxic killing when contact with a target cell that comprises a peptide-MHC (pMHC) complex selected from the group consisting of ALKDVEERV/HLA-A*02, LLFGLALIEV/HLA-A*02, SESIKKKVL/HLA-B*44, and ASSTLYLVF/HLA-B*57.

114. The host cell of any one of claims 89-113, wherein the host cell does not express MAGEC2 antigen, is not recognized by a binding protein of any one of claims 56-78, is not of serotype HLA-B*07, does not express an HLA-B*07 allele, is not of serotype HLA-A*24, and/or does not express an HLA-A*24 allele.

115. A population of host cells according to any one of claims 89-114.

116. A composition comprising a) a binding protein according to any one of claims 56-77, b) an isolated nucleic acid according to claim 80 or 81, c) a vector according to any one of claims 82-88, d) a host cell according to any one of claims 89-114, and/or e) a population of host cells according to claim 115, and a carrier.

117. A device or kit comprising a) a binding protein according to any one of claims 56-77, b) an isolated nucleic acid according to claim 80 or 81, c) a vector according to any one of claims 82-88, d) a host cell according to any one of claims 89-114, and/or e) a population of host cells according to claim 115, said device or kit optionally comprising a reagent to detect binding of a), d) and/or e) to a pMHC complex.

118. A method of producing a binding protein according to any one of claims 56-77, wherein the method comprises the steps of: (i) culturing a transformed host cell which has been transformed by a nucleic acid comprising a sequence encoding a binding protein according to any one of claims 56-77 under conditions suitable to allow expression of said binding protein; and (ii) recovering the expressed binding protein.

119. A method of producing a host cell expressing a binding protein according to any one of claims 56-77, wherein the method comprises the steps of: (i) introducing a nucleic acid comprising a sequence encoding a binding protein according to any one of claims 56-77 into the host cell; and (ii) culturing the transformed host cell under conditions suitable to allow expression of said binding protein.

120. A method of detecting the presence or absence of a MAGEC2 antigen and/or a cell expressing MAGEC2, optionally wherein the cell is a hyperproliferative cell, comprising detecting the presence or absence of said MAGEC2 antigen in a sample by use of at least one binding protein according to any one of claims 56-77, at least one host cell according to any one of claims 89-114, or a population of host cells according to claim 115, wherein detection of the MAGEC2 antigen is indicative of the presence of a MAGEC2 antigen and/or cell expressing MAGEC2.

121. The method of claim 120, wherein the at least one binding protein, or the at least one host cell, forms a complex with the MAGEC2 peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

122. The method of claim 120 or 121, further comprising obtaining the sample from a subject.

123. A method of detecting the level of a disorder characterized by MAGEC2 expression in a subject, comprising:
a) contacting a sample obtained from the subject with at least one binding protein according to any one of claims 56-77, at least one host cell according to any one of claims 89-114, or a population of host cells according to claim 115; and
b) detecting the level of reactivity,
wherein the presence or a higher level of reactivity compared to a control level indicates the level of the disorder characterized by MAGEC2 expression in the subject.

124. The method of claim 123, wherein the control level is a reference number.

125. The method of claim 123 or 124, wherein the control level is a level from a subject without the disorder characterized by MAGEC2 expression.

126. A method for monitoring the progression of a disorder characterized by MAGEC2 expression in a subject, the method comprising:
a) detecting in a subject sample the presence or level of reactivity between a sample obtained from the subject and at least one binding protein according to any one of claims 56-77, at least one host cell according to any one of claims 89-114, or a population of host cells according to claim 115;
b) repeating step a) at a subsequent point in time; and
c) comparing the level of MAGEC2 or the cell of interest expressing MAGEC2 detected in steps a) and b) to monitor the progression of the disorder characterized by MAGEC2 expression in the subject, wherein an absent or reduced MAGEC2 level or the cell of interest expressing MAGEC2 detected in step b) compared to step a) indicates an inhibited progression of the disorder characterized by MAGEC2 expression in the subject and a presence or increased MAGEC2 level or the cell of interest expressing MAGEC2 detected in step b) compared to step a) indicates a progression of the disorder characterized by MAGEC2 expression in the subject.

127. The method of claim 126, wherein between the first point in time and the subsequent point in time, the subject has undergone treatment to treat the disorder characterized by MAGEC2 expression.

128. A method for predicting the clinical outcome of a subject afflicted with a disorder characterized by MAGEC2 expression comprising:
a) determining the presence or level of reactivity between a sample obtained from the subject and at least one binding protein according to any one of claims 56-77, at least one host cell according to any one of claims 89-114, or a population of host cells according to claim 115; and
b) comparing the presence or level of reactivity to that from a control, wherein the control is obtained from a subject having a good clinical outcome;
wherein the absence or a reduced level of reactivity in the subject sample as compared to the control indicates that the subject has a good clinical outcome.

129. A method of assessing the efficacy of a therapy for a disorder characterized by MAGEC2 expression comprising:
a) determining the presence or level of reactivity between a sample obtained from the subject and at least one binding protein according to any one of claims 56-77, at least one host cell according to any one of claims 89-114, or a population of host cells according to claim 115, in a first sample obtained from the subject prior to providing at least a portion of the therapy for the disorder characterized by MAGEC2 expression to the subject, and b) determining the presence or level of reactivity between a sample obtained from the subject and at least one binding protein according to any one of claims 56-77, at least one host cell according to any one of claims 89-114, or a population of host cells according to claim 115, in a second sample obtained from the subject following provision of the therapy for the disorder characterized by MAGEC2 expression,
wherein the absence or a reduced level of reactivity in the second sample, relative to the first sample, is an indication that the therapy is efficacious for treating the disorder characterized by MAGEC2 expression in the subject, and wherein the presence or an increased level of reactivity in the second sample, relative to the first sample, is an indication that the therapy is not efficacious for treating the disorder characterized by MAGEC2 expression in the subject.

130. The method of any one of claims 120-129, wherein the level of reactivity is indicated by a) the presence of binding and/or b) T cell activation and/or effector function, optionally wherein the T cell activation or effector function is T cell proliferation, killing, or cytokine release.

131. The method of any one of claims 120-130, wherein the T cell binding, activation, and/or effector function is detected using fluorescence activated cell sorting (FACS), enzyme linked immunosorbent assay (ELISA), radioimmune assay (RIA),
immunochemically, Western blot, or intracellular flow assay.

132. A method of preventing and/or treating a disorder characterized by MAGEC2 expression comprising contacting target cells expressing MAGEC2 with a therapeutically effective amount of a composition comprising cells expressing at least one binding protein according to any one of claims 56-77, optionally wherein the composition is administered to a subject.

133. The method of any one of claims 49-55 and 132, wherein the cell is an allogeneic cell, syngeneic cell, or autologous cell.

134. The method of any one of claims 49-55, 132, and 133, wherein the cell is a host cell according to any one of claims 89-114 or a population of host cells according to claim 115.

135. The method of any one of claims 49-55 and 132-134, wherein the target cell is a cancer cell expressing MAGEC2.

136. The method of any one of claims 49-55 and 132-135, wherein the composition further comprises a pharmaceutically acceptable carrier.

137. The method of any one of claims 49-55 and 132-136, wherein the composition induces an immune response against the target cell expressing MAGEC2 in the subject.

138. The method of any one of claims 49-55 and 132-137, wherein the composition induces an antigen-specific T cell immune response against the target cell expressing MAGEC2 in the subject.

139. The method of any one of claims 49-55 and 132-138, wherein the antigen-specific T cell immune response comprises at least one of a CD4⁺ helper T lymphocyte (Th) response and a CD8+ cytotoxic T lymphocyte (CTL) response.

140. The method of any one of claims 49-55 and 132-139, further comprising administering at least one additional treatment for the disorder characterized by MAGEC2 expression, optionally wherein the at least one additional treatment for the disorder characterized by MAGEC2 expression is administered concurrently or sequentially with the composition.

141. The method of any one of claims 132-140, wherein the disorder characterized by MAGEC2 expression is a cancer or relapse thereof, optionally wherein the cancer is selected from the group consisting of melanoma, head & neck cancer, lung cancer, cervical cancer, prostate cancer, multiple myeloma, hepatocellular carcinoma, breast invasive carcinoma, and bladder urothelial carcinoma.

142. The method of any one of claims 132-141, wherein the subject is an animal model of a disorder characterized by MAGEC2 expression and/or a mammal, optionally wherein the mammal is a human, a primate, or a rodent.


SEQUENCE LISTING (81 sequences)

SEQ ID NO: 1 - protein, 9 aa, Homo sapiens
ALKDVEERV

SEQ ID NO: 2 - protein, 10 aa, Homo sapiens
LLFGLALIEV

SEQ ID NO: 3 - protein, 9 aa, Homo sapiens
SESIKKKVL

SEQ ID NO: 4 - protein, 9 aa, Homo sapiens
ASSTLYLVF

SEQ ID NO: 5 - protein, 9 aa, Homo sapiens
RAREFMELL

SEQ ID NO: 6 - protein, 9 aa, Homo sapiens
VGPDHFCVF

SEQ ID NO: 7 - protein, 8 aa, Homo sapiens
RAREFMEL

SEQ ID NO: 8 - protein, 10 aa, Homo sapiens
RAREFMELLF

SEQ ID NO: 9 - protein, 10 aa, Homo sapiens
LKRAREFMEL

SEQ ID NO: 10 - protein, 9 aa, Homo sapiens
VILKRAREF

SEQ ID NO: 11 - protein, 9 aa, Homo sapiens
FPVILKRAR

SEQ ID NO: 12 - protein, 9 aa, Homo sapiens
KRAREFMEL

SEQ ID NO: 13 - protein, 10 aa, Homo sapiens
KRAREFMELL

SEQ ID NO: 14 - protein, 11 aa, Homo sapiens
LKRAREFMELL

SEQ ID NO: 15 - protein, 11 aa, Homo sapiens
RAREFMELLFG

SEQ ID NO: 16 - protein, 10 aa, Homo sapiens
VGPDHFCVFA

SEQ ID NO: 17 - protein, 11 aa, Homo sapiens
VGPDHFCVFAN

SEQ ID NO: 18 - protein, 8 aa, Homo sapiens
VGPDHFCV

SEQ ID NO: 19 - protein, 8 aa, Homo sapiens
GPDHFCVF

SEQ ID NO: 20 - protein, 10 aa, Homo sapiens
EVGPDHFCVF

SEQ ID NO: 21 - protein, 11 aa, Homo sapiens
IEVGPDHFCVF

SEQ ID NO: 22 - DNA, 828 bp, Artificial Sequence
  Description of Artificial Sequence Synthetic polynucleotide
atggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 23 - protein, 276 aa, Artificial Sequence
  Description of Artificial Sequence Synthetic polypeptide
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - DNA, 936 bp, Artificial Sequence
  Description of Artificial Sequence Synthetic polynucleotide
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 25 - protein, 312 aa, Artificial Sequence
  Description of Artificial Sequence Synthetic polypeptide
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 26 - DNA, 816 bp, Artificial Sequence
  Description of Artificial Sequence Synthetic polynucleotide
atggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 27 - protein, 272 aa, Artificial Sequence
  Description of Artificial Sequence Synthetic polypeptide
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 28 - DNA, 924 bp, Artificial Sequence
  Description of Artificial Sequence Synthetic polynucleotide
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactca

SEQ ID NO: 29 - protein, 308 aa, Artificial Sequence
  Description of Artificial Sequence Synthetic polypeptide
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 30 - DNA, 1821 bp, Artificial Sequence
  Description of Artificial Sequence Synthetic polynucleotide
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactcaagagccaaaagaagcgggagcggtgcgacaaactttagcctgttgaaacaagccggcgacgttgaagagaaccccggacctatggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 31 - protein, 607 aa, Artificial Sequence
  Description of Artificial Sequence Synthetic polypeptide
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 32 - DNA, 828 bp, Artificial Sequence
  Description of Artificial Sequence Synthetic polynucleotide
atggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 33 - protein, 276 aa, Artificial Sequence
  Description of Artificial Sequence Synthetic polypeptide
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 34 - DNA, 936 bp, Artificial Sequence
  Description of Artificial Sequence Synthetic polynucleotide
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggacttt

SEQ ID NO: 35 - protein, 312 aa, Artificial Sequence
  Description of Artificial Sequence Synthetic polypeptide
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 36 - DNA, 1854 bp, Artificial Sequence
  Description of Artificial Sequence Synthetic polynucleotide
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaagaagcgggagcggtgcgacaaactttagcctgttgaaacaagccggcgacgttgaagagaaccccggacctatggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 37 - protein, 618 aa, Artificial Sequence
  Description of Artificial Sequence Synthetic polypeptide
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 38 - DNA, 825 bp, Artificial Sequence
  Description of Artificial Sequence Synthetic polynucleotide
atgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatacggggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgcgtcgtgtctgcccgcaacgcaggtaacatgcttacattcggagggggaacaaggttaatggtcaaaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 39 - protein, 275 aa, Artificial Sequence
  Description of Artificial Sequence Synthetic polypeptide
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSARNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - DNA, 936 bp, Artificial Sequence
  Description of Artificial Sequence Synthetic polynucleotide
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgcctgcgctagtagcttcggcaccagcggtcgcggtgaacagtttttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 41 - protein, 312 aa, Artificial Sequence
  Description of Artificial Sequence Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCACASSFGTSGRGEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 42 - DNA, 813 bp, Artificial Sequence
  Description of Artificial Sequence Synthetic polynucleotide
atgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatacggggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgcgtcgtgtctgcccgcaacgcaggtaacatgcttacattcggagggggaacaaggttaatggtcaaaccccatattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 43 - protein, 271 aa, Artificial Sequence
  Description of Artificial Sequence Synthetic polypeptide
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSARNAGNMLTFGGGTRLMVKPHIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 44 - DNA, 924 bp, Artificial Sequence
  Description of Artificial Sequence Synthetic polynucleotide
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgcctgcgctagtagcttcggcaccagcggtcgcggtgaacagtttttcgggccagggacacggctcaccgtgctagaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactca

SEQ ID NO: 45 - protein, 308 aa, Artificial Sequence
  Description of Artificial Sequence Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCACASSFGTSGRGEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 46 - DNA, 1818 bp, Artificial Sequence
  Description of Artificial Sequence Synthetic polynucleotide
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgcctgcgctagtagcttcggcaccagcggtcgcggtgaacagtttttcgggccagggacacggctcaccgtgctagaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactcaagagccaaaagaagcgggagcggtgcgacaaactttagcctgttgaaacaagccggcgacgttgaagagaaccccggacctatgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatacggggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgcgtcgtgtctgcccgcaacgcaggtaacatgcttacattcggagggggaacaaggttaatggtcaaaccccatattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 47 - protein, 606 aa, Artificial Sequence
  Description of Artificial Sequence Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCACASSFGTSGRGEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSARNAGNMLTFGGGTRLMVKPHIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 48 - DNA, 825 bp, Artificial Sequence
  Description of Artificial Sequence Synthetic polynucleotide
atgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatacggggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgcgtcgtgtctgcccgcaacgcaggtaacatgcttacattcggagggggaacaaggttaatggtcaaaccccatatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 49 - protein, 275 aa, Artificial Sequence
  Description of Artificial Sequence Synthetic polypeptide
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSARNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 50 - DNA, 936 bp, Artificial Sequence
  Description of Artificial Sequence Synthetic polynucleotide
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgcctgcgctagtagcttcggcaccagcggtcgcggtgaacagtttttcgggccagggacacggctcaccgtgctagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggacttt

SEQ ID NO: 51 - protein, 312 aa, Artificial Sequence
  Description of Artificial Sequence Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCACASSFGTSGRGEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 52 - DNA, 1851 bp, Artificial Sequence
  Description of Artificial Sequence Synthetic polynucleotide
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgcctgcgctagtagcttcggcaccagcggtcgcggtgaacagtttttcgggccagggacacggctcaccgtgctagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaagaagcgggagcggtgcgacaaactttagcctgttgaaacaagccggcgacgttgaagagaaccccggacctatgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatacggggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgcgtcgtgtctgcccgcaacgcaggtaacatgcttacattcggagggggaacaaggttaatggtcaaaccccatatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 53 - protein, 617 aa, Artificial Sequence
  Description of Artificial Sequence Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCACASSFGTSGRGEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSARNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 54 - DNA, 1122 bp, Homo sapiens
atgcctcccgttccaggcgttccattccgcaacgttgacaacgactccccgacctcagttgagttagaagactgggtagatgcacagcatcccacagatgaggaagaggaggaagcctcctccgcctcttccactttgtacttagtattttccccctcttctttctccacatcctcttctctgattcttggtggtcctgaggaggaggaggtgccctctggtgtgataccaaatcttaccgagagcattcccagtagtcctccacagggtcctccacagggtccttcccagagtcctctgagctcctgctgctcctctttttcatggagctcattcagtgaggagtccagcagccagaaaggggaggatacaggcacctgtcagggcctgccagacagtgagtcctctttcacatatacactagatgaaaaggtggccgagttagtggagttcctgctcctcaaatacgaagcagaggagcctgtaacagaggcagagatgctgatgattgtcatcaagtacaaagattactttcctgtgatactcaagagagcccgtgagttcatggagcttctttttggccttgccctgatagaagtgggccctgaccacttctgtgtgtttgcaaacacagtaggcctcaccgatgagggtagtgatgatgagggcatgcccgagaacagcctcctgattattattctgagtgtgatcttcataaagggcaactgtgcctctgaggaggtcatctgggaagtgctgaatgcagtaggggtatatgctgggagggagcacttcgtctatggggagcctagggagctcctcactaaagtttgggtgcagggacattacctggagtatcgggaggtgccccacagttctcctccatattatgaattcctgtggggtccgagagcccattcagaaagcatcaagaagaaagtactagagtttttagccaagctgaacaacactgttcctagttcctttccatcctggtacaaggatgctttgaaagatgtggaagagagagtccaggccacaattgataccgcagatgatgccactgtcatggccagtgaaagcctcagtgtcatgtccagcaacgtctccttttctgagtga

SEQ ID NO: 55 - protein, 373 aa, Homo sapiens
MPPVPGVPFRNVDNDSPTSVELEDWVDAQHPTDEEEEEASSASSTLYLVFSPSSFSTSSSLILGGPEEEEVPSGVIPNLTESIPSSPPQGPPQGPSQSPLSSCCSSFSWSSFSEESSSQKGEDTGTCQGLPDSESSFTYTLDEKVAELVEFLLLKYEAEEPVTEAEMLMIVIKYKDYFPVILKRAREFMELLFGLALIEVGPDHFCVFANTVGLTDEGSDDEGMPENSLLIIILSVIFIKGNCASEEVIWEVLNAVGVYAGREHFVYGEPRELLTKVWVQGHYLEYREVPHSSPPYYEFLWGPRAHSESIKKKVLEFLAKLNNTVPSSFPSWYKDALKDVEERVQATIDTADDATVMASESLSVMSSNVSFSE

SEQ ID NO: 56 - DNA, 1089 bp, Homo sapiens
atgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttga

SEQ ID NO: 57 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 58 - DNA, 1098 bp, Homo sapiens
atggccgtcatggcgccccgaaccctcgtcctgctactctcgggggccctggccctgacccagacctgggcaggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctactacaaccagagcgaggccggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcagctcagatcaccaagcgcaagtgggaggcggcccatgtggcggagcagcagagagcctacctggagggcacgtgcgtggacgggctccgcagatacctggagaacgggaaggagacgctgcagcgcacggacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatgggagccatcttcccagcccaccgtccccatcgtgggcatcattgctggcctggttctccttggagctgtgatcactggagctgtggtcgctgctgtgatgtggaggaggaacagctcagatagaaaaggagggagctactctcaggctgcaagcagtgacagtgcccagggctctgatgtgtctctcacagcttgtaaagtgtga

SEQ ID NO: 59 - protein, 365 aa, Homo sapiens [sequence omitted]

SEQ ID NO: 60 - protein, 20 aa, Artificial Sequence
  Description of Artificial Sequence Synthetic peptide
  SITE (1)..(10): This region may encompass 0-10 residues
  SITE (11)..(20): This region may encompass 0-10 residues
GGGGGGGGGGSSSSSSSSSS

SEQ ID NO: 61 - protein, 10 aa, Artificial Sequence
  Description of Artificial Sequence Synthetic peptide
GGGGSGGGGS

SEQ ID NO: 62 - protein, 8 aa, Artificial Sequence
  Description of Artificial Sequence Synthetic peptide
GGGSGGGS

SEQ ID NO: 63 - protein, 11 aa, Artificial Sequence
  Description of Artificial Sequence Synthetic peptide
GGGSGGGSGGS

SEQ ID NO: 64 - protein, 9 aa, Unknown
  Description of Unknown “LAGLIDADG” family peptide motif sequence
LAGLIDADG

SEQ ID NO: 65 - protein, 66 aa, Homo sapiens
SESSFTYTLDEKVAELVEFLLLKYEAEEPVTEAEMLMIVIKYKDYFPVILKRAREFMELLFGLALI

SEQ ID NO: 66 - protein, 66 aa, Homo sapiens
KYEAEEPVTEAEMLMIVIKYKDYFPVILKRAREFMELLFGLALIEVGPDHFCVFANTVGLTDEGSD

SEQ ID NO: 67 - protein, 66 aa, Homo sapiens
YFPVILKRAREFMELLFGLALIEVGPDHFCVFANTVGLTDEGSDDEGMPENSLLIIILSVIFIKGN

SEQ ID NO: 68 - protein, 66 aa, Homo sapiens
EVGPDHFCVFANTVGLTDEGSDDEGMPENSLLIIILSVIFIKGNCASEEVIWEVLNAVGVYAGREH

SEQ ID NO: 69 - protein, 9 aa, Homo sapiens
VGLTDEGSD

SEQ ID NO: 70 - protein, 9 aa, Homo sapiens
TVGLTDEGS

SEQ ID NO: 71 - protein, 9 aa, Homo sapiens
NTVGLTDEG

SEQ ID NO: 72 - protein, 9 aa, Homo sapiens
ANTVGLTDE

SEQ ID NO: 73 - protein, 9 aa, Homo sapiens
FANTVGLTD

SEQ ID NO: 74 - protein, 9 aa, Homo sapiens
VFANTVGLT

SEQ ID NO: 75 - protein, 9 aa, Homo sapiens
CVFANTVGL

SEQ ID NO: 76 - protein, 9 aa, Homo sapiens
FCVFANTVG

SEQ ID NO: 77 - protein, 9 aa, Homo sapiens
HFCVFANTV

SEQ ID NO: 78 - protein, 9 aa, Homo sapiens
DHFCVFANT

SEQ ID NO: 79 - protein, 9 aa, Homo sapiens
PDHFCVFAN

SEQ ID NO: 80 - protein, 9 aa, Homo sapiens
GPDHFCVFA

SEQ ID NO: 81 - protein, 9 aa, Homo sapiens
EVGPDHFCV